SEQUENCE LISTING (6 sequences)

SEQ ID NO: 1 - DNA, 38786 bp, unidentified
  source 1..38786: mol_type = genomic DNA
gctgaccgatgaacaggtggcctacattgtgaactccaagaagagtagccgcgagttagccaacgagtttggagtctcgccgcaggctataaactggaggaagaaacaatggcaggctacggcgcaaaaggaatccgaaaagtaggttccttccgttctggacttgaagataaagtccacaaacaactggaggctcgtggtataaaggctgaatatgaaatgtggaagattccatatgttgtgccagccagtaatcacacgtaccgaccagattttattttacccaatggaatcattgtcgagaccaagggactgtgggaagctgatgaccggaagaagcacttgctaatccgtgagcagtatcctgaactggacattcgtctggtgttctcttcgagtcgtaccaaaatctacaaagggtctccaacttcttacgccgagttctgcgagaagaaagggattctctttgccgacaaactgatacccgtcgagtggctcaaggagcctaagcgggacgtaccgtgggagaaactaattcgtgtaaaggagaaaaagtaaatggctaaagtacaattcaaaccgagagcaaccaccgaggcaatcttcgtgcattgctctgcgactaaaccaagtcagaacgtaggcgttcgagagattcgccagtggcataaagagcaaggttggctcgattgcggatatcacttcatcatcaagcgtgatggtactgtggaagcaggccgtgacgaactggctgtaggttcccacgccaagggctacaaccacaactctattggtgtctgccttgtaggtggcatcgacgataaaggtaagtttgacgctaactttactccagcccaaatgcaatctctccgctcactgctggtcacactgctggcgaagtacgaagggtctgttctccgtgctcaccacgatgttgcacccaaagcctgcccgtccttcgacttgaagcgctggtgggagaagaacgaactggttacatctgaccgagggtgataccatgaggatgatttgcattcgttgtgacaagcctttgaagagtcgcgtaccatcacgtcagtgtcagtgcaaagacccaatcaaggcggaggaacacaccgagaagtaattaacactcactaaagggatagtcgaaaggccgtccctttgttcgcactattgattaaggattaaccacatggaacaagaaaacgacagtatattcctttaccatatcccgtgcgagaactgtgggtcttctgatgctaactcattgttctccgatggtcacacgtactgttacgcctgtgagaatcggacgccgggggatgaacggaaagctgagcagttatcaacgagaagacgtacaggaggcagcaagcctatgagctatgacgtatggaatttcggtgacagcaacggtcgctactctgacctgactgcccgtggcatctcgaaggagacatgccaaaaggctggctactggctggcgaaagtggacaaccgaatgtatcaggttgccgactaccgagaccagaacggctccatcgtgtcacagaaggtgcgcgataaggacaagaactttaagaccaccggaagtcacaagagcgatgcattgttcttgaagcacctctggtctggcggtaagaaaattgtggtcactgagggcgaaattgacgcgctcaccgtgatggaactccaagactgtaagtacccggtcgtatcattgggtcacggtgcctctgctgctaagaagacatgcgccgctaactatgaatactttgaccagttcgagcagattatcttgatgttcgacatggatgacgcaggccgtaaggcagtcgaagaggccgcacaggttctcccggctggcaaggttcgtgttgctgtgttaccgtacaaggacgccaatgagtgtcacatcatgggtgaagacaaagcaatcttggagcaggtctggaacgcgaacccttgggtgcctgatggtgttgtctctgcgttgtcactcaaagaccgcgttaaggaagctatgacctccgaggacgctgtaggtttattattcgatggctgtcaaggcctgaacgatagaaccttgggtgcccgtggaggcgaagtcatcatggtcacttccggttccggtatgggtaagtcaacgttcgttcgtcaacaggcgctggcttggggcaagcgaatgggtaaacgtgttggcctagcgatgctggaggaatccgttgaggataccattcaggacatgatgggcttgaacaataaggttcgtctgcgtcagtctgatgaagtcaagaaggctatcgcagaggacggacgtttcgatgaatggtatgacgaactatttggagacgatacgttccacctttatgactcctttgcggaggccgaagctgacaggctgttagcgaagctggcctacatgcgaacaggcttagggtgtgatgttatagtgctcgaccacatatcaatcgtcgtgtctgcctctgaggaatcggatgagcgtaagatgattgaccgcctcatgactaagctaaaagggttcgctaagtcaactggtgttgtgctcgtagtgatttgtcacttgaagaacccggagaagggaaaagcacatgaagaaggacgcgctgtttctattactgacctacgcggttctggtgccctgcgccagctttctgatactatcatcgccttggagcgtaaccagcaaggtgatatgccacatctggttcttgtgcgtttgcttaagtgtcgtttcactggcgatactggaattgctgggtacatggagtacaacagagaaacaggatggcttgaaccatctagctatactggagaagaaggagaaggagatactggctggactgaacaagacggacagtcagacttttgacgaatggcaggaactaaatgagcgcgaacaggcgctctattcctccaagaagtaattaaccctcactaacgggaataacctcaaaccataggagacacatcatgtttaaactcattaacgctttaggtaagctggtagttaaactgtacttcatcgaagccaagaagctggacaagaaggccaaagctgattcacagcgagccattgagttggcgaaacagtcccgtgagaaatccgatgcggctgttagtggcatccacaagtcggcagcgattgcagccaaagcacagtccatgagcaaattctttgagtaaggagcgcacactatgagcaagtcaatcacccacgcaaacaccatccgtcttccagacaccgctgaccagttctcccgccgagtacacatcaatgtgcgtggtgagaaggtcacgatggtctaccgctggaaagaccacaagtctccgaaagcgcacactcaacgcatgaccttggatgacaagcaggtaggtcgcctgatgggtgccctgactatggctgctgacaaggtggtcggtgataaccgtgagcgtctggtagagttcggcgctggtatgcaagagattatcgagaagtgataaacccaaggccgcccttgggcgacctttatgattatcatttagcacgaaaccaaaggagggcattatgctcgtaagtgacattgaggccaacaacctcttagagaaagtcaccaagtttcactgtggcgtcatctatgactaccgtgatggtgagtatcatagttatcgtccgggcgacttcggggcgtatctggatgcgctggaagccgaggtgaagcgaggtgggcttattgttttccataacggtcacaagtatgacgttccggctttagagaaactcgcaaagttgcaactcaatcgtgacttcaagttacccagtgagaattgtattgacacattggtactctcgcgtttacttcatgcgaaccttaaagacacagacatggggcttcttcgttccgggaaactacccggtcgtcgcttcgggtctcacgctctggaggcatggggttatcgcttgggcgagatgaaaggtgaatacaaggacgacttcaaggctatgctggaagagcagggcgaagaatatgttgaaggtgccgagtgggtttcgttcaacgagccgatgatggcgtataacgttcaagacgttgtggtcactgtcaagctcttcgagaaattactgagtgacctccactacttccctgcgggtatggacttcacgaagtacgatgcggatttattctggcgagaagctggcgagtctgttgatattgaacatcgtgcagcgtggctattagcgaagcaggagcgtaacggcttcccgttcaacaccaaggccattgaggaactttatgtagaactggcagcgaaacgcgccgaacttctacgaaagttaaccgaaacgtttggctcgtggtatcaaccgaaaggtggtaaggaactcttcaagcacccacggacaggcaagccgctgccgagttatcctcgcgtggtctatccgaaacagggtggcgtgtacaagaagcctcgtaataaggcacagcgcgaaggtcttgaaccttgcgacttggacacgcgggattacatggagggtgctccgtacactccggtcgagttcgttacgttcaagccaagtagccgtgaccacattcagaagaaactacaggaggccggatgggtgcctgagaagttcaccgagaagggtgcgcctgtggtggacgatgaggtcttggagcatgtgcgagtggatgaccctgagaagcaggcgtgcattgacttgattaaagagtacctgatgatacagaagcgtatcggtcaggcggcagaaggtgacaaagcgtggttacgttatgttcaagatgatggtaagattcatggagccgttaaccctaacggtgcagttactggtcgtgctactcatagtttcccaaaccttgcacaagtacccggcgttcgttctccttacggggagcagtgtcgtagcgcttttggcgctgaacatcatttggatgggattactggcaagccgtgggtgcaagcggggatagacgccagtggcttagagttgcgctgtctggcacacttcatggctcgcttcgataacggagagtatgcgaatgagattctcaacggtgacattcacaccaagaaccagaacgctgcggaactcccaacgcgagacaacgccaagaccttcatttacgggttcctttacggagcaggagatgagaagattggtcagattgttggggccgggaaggagcgcgggaaggaactcaagaagaaattcttagagaacaccccggcgattgcagcgctacgagaagctatccaacagtctctcgttaagtcttccgcttggattggtggtgagcagaaagtccagtggaaacgccgttggattaagggtctggatggtcgtaaggttcacgtcaggtctccacacgcagcgctcaacacattgttgcagtctgctggtgctctgatttgtaagctgtggattatcaagaccgaagagatgctcattgagaaaggcttgaagcatggctgggatggtgactttgcgtacatggcttgggttcacgatgagattcaggtggcctgccgtaccgagaacatcgctcagattgttattgacaccgcgcaggaagctatgcgttgggtcggagagcattgggctttccgttgtcgtttagataccgaaggtaagatggggccgaattgggccgtctgccactaacagtaggagaaacattatggcaatgaccaaacactttaaagtaaccttcgacgttaccgctgtgattgacagtgaatccgagaagaatctgaacgagttgctggtagacctagctaagaaggccggggctggagagaagctgaacccgttcaaacaagaactgcttattcaaggtctgacacacgggccggaaggcgctgctgcgttctgtatcaagcaaggtctccgtgagtttatccgtgatggtcacaatgagttaagctctaccgagcagaagttgatgagattctcacctgcaactgtgcgcgaggtgttcaaatgagtgaatacctgaaagttctggcagctatcaaaggttgccctaagtcctttcagtcgaactatgtgcgaaacaatgcggctctcgtagctgaggccgcttctcgtggacacattagctgtttgactgtgggtggtcgcaatggtggagcatgggaggtcacggcctctggtgtgcgcttcctgaaacgaatgggaggttgcgtatgattatgccaaagagtgacaccgtaacgatgacccgtgatgcgtggaacgatgttagcgcgtacattgataagctggagaaagacccagagttcctgaatgcgcttaaggcgtgtggtgtggataattgggacggatactctgatgcaattgaaatggtttacggggaggatgacgaatgagcctaatcacattgaaagacttcgcggagatgcgtgaaggtaagcctgtggagaagggagtgctggtcatggatggtgactggttggtatatcagtcaatggctgctgccgaagttgagacggattggggagatgacatttggactcttgagtgtgaccacgccaaggcacggagcattctcgattctgcaatcgaatcgtaccgaacccgtaagaaggcttggagtgacgctatggttgttctggcgtttaccgacgatgtgaactggcgtaaggtactggttgatgagacctataaggagaaccgcaaggcgacccgtaagcctgtgggctaccgtgacttcctgtcgaagctgtgggagcgcgatgagttcattcacatcaaagaggacatgctggaaggtgatgacgttatgggcatcattgggtctggtcacgaggtcttcggcttcaagaaggctgtgttggtctcctgtgacaaggacttcaagactatcccggacgttgacttcctgtggtgtaccactggtaacatcttgacgcagaccaaagagaccgctgattggtggcatctcttccagactatcaaaggtgatatgactgatggctactccgggattcccggctggggcgatacggcagaaggtttcctgaatgacccgttcattgtggagcctgtagagtccgtgttgaagtccggcaagaacaaggggcagacagtaaccaagtgggtgaagcgtgctcctgatgcgactgagacgctgtgggactgcattaaatccattggtgccaaagctggtatgaccgagcaggaaatcatcaagcaaggccagatggcccgcattcttcgcttcgaggaatacaattacattgacaaggagatttacttatggacgccgcgaagctgattaccgtcgctgtgctggtaggtatgctggcgcgaggtctctggatgttcgctctcatagtccgagacgtgaagcgagactccgagattaagtgataaactcaaggctctctgtattaaccctcactaaagggaagagggagcctttatgattattactttaagacttaaccttaagaggtgaaattatgttacaaccaattaatcatgttttaactcatcccgacgatattcctagtatgcctcgtgcagctaaagagtatctacaggtgcgcttcaatcagacctatgtgatggagtctggggagtaccgtgcgctacgcgctgctggatactctgagtctttcattgcgggtgtgatgcatggtctgtaccttgcctctcgaacacttgatgagattgaggtgcgtaaagagcaactgagacaggagtgattatatgtgctggaaaccgaaggtgaaagtccctaagatggacactaaccagattcgcgctgccgaaccagcacctttgactgaaccaccgaaatctgtggtatggggaggtgatgatgacgaggacaccagcgtgtcttcctctgaggtgccaacctcaccgagcagcggtaagagcagtctcaaggtgaagctggatgacagcgcagccaagaacaagagtaaatcctccattcgctccaaggcgtttgggtaactgagtcatagcatagggagaaacatcatgggattcttcaagaaaatcaagaaggctgttaagaaagtcgtgaaggaagttagcagaccagtcgagaaggcaggtaaggaagttggtaaggttgttggaggcgcgcttggtgctggtaagcaggagattatccaacaagaggctccagtaccagtagtcgctgcaccgccaccagcacaggttgtagatgtaccggaacaggataaggctgagggtgaggatgaggcgcaaaccgaaagcgcacgtaagaaagcacgcgctggcggtaagaagtctctgagtgtagctcgtagctccggcggtggcatcaacatctaaggaggactgaatggctgattcaaaacgtacaggattaggcgaagatggtgctaaagctacctatgaccgcctaacgaacgaccgtagagcctatgagactcgtgcggaaaactgtgcgcaatacaccattccgtccttgttcccgaaggagtccgataacgaatctaccgactacacgactccgtggcaggctgtaggtgcgcggggtcttaacaatctggcctctaagttaatgcttgcgttattcccgatgcagtcgtggatgaagctgaccattagtgaatatgaggcgaagcagcttgttggagaccctgatggactcgctaaggtggacgaaggtctgtctatggttgagcgtattatcatgaattatatcgaatccaacagctaccgtgtgacgctctttgagtgcctcaagcagttgatcgtggcaggtaatgcactgctttacttaccggaacccgaaggtagctacaatccgatgaagctgtaccgattgtcttcttatgttgtccaaagagacgcatacggcaatgtgttacagattgtcactcgtgaccagatagcctttggtgctctcccggaagacgttcggtctgcggtagagaaagctggtggtgagaagaagatggacgaaatggtcgatgtgtacacccatgtgtatctcgatgaagagtccggcgattacctcaagtacgaggaagtagaggacgttgagattgatggctccgatgccacatatccgactgacgctatgccctacattccggttcgcatggttcgcattgatggcgagtcttacggtcgctcctactgtgaagaatacttaggtgacttaaggtcgcttgagaatctccaagaggctatcgttaagatgagcatgattagcgctaaggtcatcggtctggttaacccggctggcattacgcagccccgtagattaaccaaagctcagactggtgacttcgttccgggtcgtcgagaagacattgacttccttcaactggagaagcaagctgactttaccgtagcgaaagctgtgagtgaccagatagaagcacgcttatcgtatgcctttatgttgaactctgcggtacagcgcacaggtgaacgtgtgaccgccgaagagattcgatacgttgcgtcagaactggaagatacgcttggtggcgtctattcgattctgtctcaagaactgcaattgcctctggttcgtgtgctcttgaagcaactccaagcaacctcgcagattcctgagttaccgaaagaagccgttgagcctactatcagtacaggtctggaagcaatcgggcgtggtcaagacctcgataagctggagcgctgtatctccgcttgggctgctctggctcctatgcagggagacccggacattaaccttgctgtcattaagctgcgcattgctaacgccattggtattgatacttctggtatcctactgacggacgagcagaagcaagcactgatgatgcaggatgcggcccaaacaggtatcgagaacgctgcggctgctggtggtgctggtgttggcgctttggctacctcaagtccagaagctatgcagggtgcagcagcacaggctggcctagacgccacctaataaccctcactaaagggagacctcatctttgaaatgagcgatgactaaaggttggagtcctttggtttccctttatctttaacaacttaggagatttaacaatggctgaatctaatgcagacgtttatgcgtccttcggtgtgaacaacgcggtaatgaccggaagcacacctactgaacacgaacagaatatgctgagtctcgatgttgctgcccgtgatggcgatgatgcaatcgtacttagcgaggaaccgaggtctcataacgatgacccttatgcggctggtgaagacccattcgctgagggcgaggatgatgagggccgtattcaggttcgtatcagtgaagatggtagtgaagccgagttcgacactggtagcgataatgctgaggtggagaccgaaggtgaagccgctgagtttgagccgctgggtgacactccagaagaactgagccaagtgactgaacaattaggtcagcacgaagaaggctttcaggcgatggtcgagcaggccgttgagcgtggactgagcgcagagtctgtgactcgcatctacgaagagtatgaagccgatggtatctctgagaaatcctacgcggaactcgaagctgctggctatagtcgtgcctttgtggactcctacatctccggtcaggaagccctcgtagaccagtacgtcaatcaggtagttgcctttgctggtggtaaggagcgctttagcgcaatccatacgcaccttgaagcaaccaacccggatgcggctgagtcccttgaggctgccatgatgaaccgagacttggcgactatcaaagcgattatcaatctggctggtgagagttacacgaagaaattcggtaagcccgccaaccgtagtgttaccaagcgtgctactccggttaaacctgtagctcgtcagaaagagggctttacgaatcaggctgagatgattaaagctatgagtgacccgcgttaccgtagtgatgctgcctaccgtcaaatggtagaacagaaggttatcgactccagtttctaattaaccctcactaaagggagagaccatagatgactacaatggttgaatcacctgagcacagaactttgtggtcactcccgtaggtgaaacattgagaaccaactcgattcaagtagtaaccaaacttttctttaaattaacataaggagattcaacatggctaacattcaaggcggacagcaaattggtactaatcagggtaagggtcagtccgcagcggacaaattggcgctgttcctgaaagtgttcggcggtgaagtcctgacggctttcgctcgcacctccgtgaccatgcctcgtcacatgctgcgctctattgcttctggtaagtccgcacagttccctgtgattggtcgtaccaaagctgcttacctgaaaccgggtgagaacctcgatgacaaacgtaaggatatcaaacacaccgagaaggtaatccacattgatggcctgctgactgcggacgttctgatttacgacattgaggacgcgatgaaccactatgacgttcgcgccgagtacaccgcacagttgggtgaatctctggcgatggcagcggatggtgctgttctggctgaactggctggtctggttaacctgcctgacgcctccaatgagaacattgagggtctcggtaagccgaccgttctggaactcgttaagccgacctctggcgacctgactgacccggttgagctgggtaaagcgattatcgctcagttgaccgttgctcgtgctgctctgaccaagaactacgttccggctgctgaccgtactttctacaccactcctgataactactccgcgattctggctgctctgatgccgaacgctgcaaactatcaggcactgattgaccctgagcgcggtactatccgtaacgtgatgggcttcgaagtggttgaggttccgcacctgactgctggtggtgctggtgatgaccgtgaggacactccggctggccagaagcacgctttcccggcgacttccagcactaccgttaaggttgctctggataacgttgtgggcctgttccagcaccgctctgcggttggtacggtcaagctgaaagacttggcactggagcgcgctcgtcgtgcgaactatcaggctgaccagattatcgctaagtacgcgatgggtcacggtggtctgcgtccagaagctgctggcgctatcgttctcccaaaggtgtcggggtaattcccgaccccactggagttactcttagtcagaagactatgagcttagacgttggggcttctaaagcactgactgctacggtaactccggctgatacagtacagacagtgacctttacgtcttccaatgcgaagattgctacggtcgatgcctctggtactgtaacggctgtgggcgctggctccgctgacgtcactgcgactactgtgaatggccttacggctgtctgcaaggtcactgtgaaagcagtagagcctcccgtagaaggctaaggtctttaccggggatttactttccttcattactttatgaaccccttgggtgccctctgagggtatctgaggggttttttgctttaaaccctcactaacaggaggtaacatcatgcgctcttatgagatgaacattgagaccgcagaagagctatcagccgtcaacgacattctggcttccatcggtgagccgccagtatcgacccttgagggtgatgcgaatgctgacgttgcgaatgctcgacgtgtactcaataagattaaccgacagattcaatcacgaggatggacattcaatattgaggaaggtgtgactcttctaccggatgcgttctctggtatgattccatttagctctgactatctgtccgtaatggcaaccagcggtcagacccaatatatcaaccgtggtggctatctctatgaccgctctgcgaagactgaccgattcccttctggtgttcaggtcaacctgattcgcctgagagagttcgatgagatgcctgagtgcttccgtaactacattgtcaccaaggcttcccgccagtttaacaaccgcttcttcggtgcgccggaggtagacggagtgttgcaggaagaagaacaggaagcgtggcgtgcgtgcttcgagtacgaactagactacggcaactacaacatgctggacggtgacgcattcacctctggtctacttaaccgctaataacaaggaggctctatggctctcattagtcaatcaatcaagaacctcaagggtggtatcagccaacagccagatattctccggttcgctgaacaaggtagcgtacagattaacggttggtcttctgagtccgagggtctccagaagcgcccaccgatgattcaccttaagacccttggtgctgctgggtatgtgggtgcgcaaccttacgttcacctcatcaaccgtgatgagttcgagcagtatttcgtggtgttcactggtgaagacattaaggtgttcgacctagatggtaaggagtatcaagtccgtggagaccgctcttatgtgcgcacagctaacccacgagaagaccttcggatggtaacggtggcagattatacctttgtgactaaccgcaaggtggttgtgcagagtaacgaccaatcggtcaaccttccgggttttaaagaccaaggcgatgcgctgattaatgttcgtggtggacaatatgggcgtagattatcaatcgagttcaacggggctgagcgtgctgctgtgcaactaccagatggttcacaaccagcacacgttaacgaggtggacggtcaggctatcgctgagaagctggcggtgcagttgaggaacaaccttgggaatccaaacaacgagcaagacccgaataagtggcgcttcaacgttggccccggattcatccatatccttgcgccaaataatgataacgtttggggactacagactaaggatggttacgcagaccagctaatcaaccctgtaacccattacactcagtcgttccagaagctgcctattaacgcgccagatgggtacatcgtgaagattgttggcgatacctcaaagactgccgaccagtattacgttaggttcgacctcaaccgtaaggtgtgggtagaaacaatcggctggaacacaaggactcacctgcactaccacacgatgccttgggcgttggtacgtgcttctgacgggaactttgacttcaagtaccttgagtggggtgctcgtactgttggtgatgatacaaccaacccatacccatcgtttaccggacagacaatcaatgatattttcttcttccgtaaccgtttggggttccttagtggggagaacatcatcttgtctcgtacctcgaaatacttcaacttcttcccggcctccgtgtcaaactactccgacgatgacccaatcgacgttgcggttagccacaaccgggtgtcaaccttgaagtatgccgttccgttctccgaagagttgctcctgtggtctgaccaagcgcagttcgttctgacagcctctggtatcctatcgagccgctccgtggagttgaaccttacgacgcagttcgacgtgcaagacagggcacgaccacatggtgtaggacgtaatgtatactttgcgtcacctcgcgcttccttcacatccattaaccgatactatgcggtacaggatgtaagctctgtgaagaacgcagaggacatgacggctcacgttccgaactacataccgaatggagtattcagtatatcggggacaaccgctgagaacttcgcggctatcctcacgagtggtgcgccgaaccgagtgtatatctacaagttcctctatattgacgaggaaatcaggcagcagtcgtggtctcattgggactttggggacaacgttacggtgtttgcagcacaggtgataaactcaacaatgactgtattgatgagtaatgaacatgctgtgtggatgggacgcttacacttcacgaagaactccatagatattccgggagagccttacaggttatacatcgacgctaagaggaaatacaccatccctgctgggacgtataatgacgatacctaccagacgtcaataagcctcgcaacaatttatgggatgaacttcactaggggtagagtgtctgtggtctttcctgatgggaagattgtagagatagaccagccaatcaacgggtggagcagcgacccaatgctgagacttgatggaaaccaagagggacaagtggtttatatcgggttcaacatcccgttcacttatacgttctcgaagttcctcattaagaagactgctgaggacggctcgacggctaccgaggatatcgggcgtttgcagcttcggcgtgcatgggtaaactatgaggactctggagcgttcaccatccgtgtgaacaacctgtcccgtgagttcatctacacgatggctggtgctcgcttgggttctgacaatctgcgtgttggtgggtcgaacattggcacagggcagtaccgcttcccggtggtgggtaatgctcagacgaacatagtaaccatcgagtcagacgcatcaaccccgctgaacatcattgggtgcggttgggaggggaactaccttcgtcgttcttctggcatttaacttaaagtctccctgtggtgaattaaccctcactaaagggagacactaatagatacgaggggttaaagcattatgtacattacaaagtgtttacaaatccatgccgacaactttcagccgtccatagaggacattctggaagctgaggcactaggtgttgaaccaaaggtaattcccgatgagaacactgtggcaatgcttagcgataacgctgtgttggcaatcggtggcaatcagggagaccgagtgtggttccttacctctaagtacgtcccactgtttaccttgaaggagcgcttagagtttcgtagacttatcattgaatatcgtgatatgatgctgagccagtacgagtccatttggaatttcgtttgggttggcaataagtcacacattcgtttcctaaagaccataggcgcggtattccataatgagtttaccgctgatggtcaattccaattattcactatcagtaggaggtaactatgtgctggatggcagcgattcctattgctatggctggtgcccaagctctaagtagccagaacagtgctgacaaggcgcgagtggcgcagaccgaagctgggcgcagacaggcaatggagatggtcaaagagatgaatatccaaaatgccaacgcctcgctggaacaacgggacgcccttgaagctgcatcctctgagttgactacacgtaacatgcagaaggtacaggctatgggaacaatccgtgcggctatcggcgagagtatgctagaaggtgaatccatgaagcgcatcaagcgtatcgaagagggtaactacatcagggaggcaaatagtgtcaccgagaattaccgccgagactacgcgagtatctttgcgcaacagttgggacgcactcagtcaacagcaagccaagtcgattccatatacaagagcgaggccaaaggtaagtccggtctgatgcgtgtactagaccctctgtccattatgggtcaggaagctgcaagtcaatatgcggctggtggatttgacaagaaaggcggcaaccaagcagcacctatcagtgccgccaaaggaaccaagaccgggaggtaataatggctagtaaactaaatagtgttttaggcaacatggcgactcccggtatggaacgactccggggagtcaaaggtatggactaccgggcagcaaccattcaggctgaacaaccaagagcgagtcttctggactccctaggtcgattcgctaaggctggtgccgatatgtacatggcgaaagaggaacgtgataagcaacgagccgatgagcgctccaacgagattatccgtaagctgacccctgagcagcgccgacaggctatccagaatggtacactgctgtatcaggatgacccatacgcaatgcaagccttgaagttcaagacagggcgtaacgctgcgttcctcgttgacgatgaggttcaacaggctatcaaagagggtcgcttccgtactcgtgaagagatggagcagtaccgacactcacgcctacaggagcactcgaagtcgttcgctgagcagttcggcatcaatgagatggacgaggaataccagaagggtttcaatgcgaatatcaccgagcgtaacattgctctgtatggggcacatgataccttcctgagtgaccaagcgcagaagggtgctatcctgaactcgaaggttgaactcaatggtgtcctgtctgaccctgacctactatctcgtcctgagtccggtgagttcttccagaactacatcgacaactccttggtgacagggatgactgacaatcaggctcaacaggttatctctgcgtccctgaatgacgtgctacagcgtccgggtggtgccgccttcttgcagaacattgagaaccgcaaggtgactcttaatggcgctacgactacctatcgtgaactgatgggtgatgagcagtggcaggcaatgatgattaaggcacagcacactcaattccagaacaacgcgaagttaaccgagaagttccgactggatattaactcagcgcttaaccaagcggacactggcaaaggctgggagactcttcaaggcatcaaggccgaactcgataagattcagccgggtgaagaggtgactcctgagcgtgagtggttgattgccgcacaggaacagatgcgcacccgcttcaagcaggaacaggctgagacagccaagctgatggacaagcaacagaagaccctgaataaatccttggtcatcgacaagcagttccagaagcgcctcaatggtgaatacgtctcgaccaactataaggacatgccgaccaacgagaacactggtgagtttacacatagcgatatggtcaactacgcgaaccagaagttagcggaaatcgaggcgatggacttaacgccagaacagaaagacaaactgaaattggactacctacgggcagactccaaggagggtgctttccgtgcagcgtttggtgagatgattactgatgctggaaacgaatggcaggctgcggtgattaacggtaagatgcctgagagcacaccagcgatggacaaccttcgtcgcatccgtaacactgaccctgacctgattgcctctctgtatcctgacaaggccgagttgttcctgacgatggacatgttggataaccaaggtatcgacacgcaggttcttatcgacgctgataaggcccgcgcacagaagaccaaggagatgcagtttgaggatgaccgagcgtgggccgctatgatgaacgattcaacgaatcctcaaatcaagtacatgccttcgagtgtccaaggttacgcgcggaagatttatgattccgtgaagtaccggacagggaaccctgacatggcaaccgaacaggtcgcaaagttcctctccgattcgacgaccacgttctccagtagcgatgttgagggtgatacctacggcgtcctaccgaagaacatcctgactgtttcggatgaccctaagtcgtgggagcaagggaagaacattctggacgaagccattaagggaatcacagcggcgaacccttggattaccaacaagcaactgtcagtgtaccagcaaggtgattcaatctacctgatggacacaaccggacaggttcgcattcggtatgaccaagagttactcaaacgcgaataccagcgtaccgcacagatgcaagctgaggcagcgcgagagaaagctctcaaagaggcaaacaaacgtgccccaatcagtcgtgcaacacaggcgcgtgaagaagcgcgtaaacgtgtccaagagaaacggaagaagactccgaagtacatctatggacgtaaagaagactaacagtgataaggaggctccatgagctacgataagaacaaacctagcgagttcgatgggttatttcaaaaagcggcagacaatcatggggtctcctatgacctgcttcgtaaattagcattcaacgaatccagctttactccaaaggccaaatccccaactggcccgaagggtctcatgcagttcaccaagggtacagccactgcgctgggactcaaggtcactgatgcagacgatgatgaccgttacaacccggagttggctgtggatgcagccgctcgtcacctgagtgacctcattcgtaaatacgatggggatgagcttaaggccgccctagcgtacaaccaaggcgagggcagaaatggtgcccctcagatgcaggcctacgacaaaggtgattgggcctcaatctctgaggaaggtcgtaactatatgcgcaacctgatggacgtagctaacagtcctcgcaagggtgacttggaggcgttcggcggcatcaccccaaaggctaagggcattccatcgggggatgcatttgcgggaatcgggaagaagcagacggtaggaactgacctaccggagtcaactggattcaaagtggagggtaaagagcagaaagcacctaacgtgccctatgctaaggacttttgggaaaagactggaacaacgctggacgagtttaattcacgttcgaccttcttcggaattggtgatgcaacaagtgcggagcttcacaactctgtacttggagttgctttccgtgcagcccgtagcgacgatggttttgatttgttcaaggacaccattacgcctacccgttggaatagccatacgtggactccagaagagctagagcgaatccgtaaggaagtgaagaacccatcgtacatcaacgtagtcactggaggttctcctgagaatctggacgccctcattaagatggcgaatgacaactacgagatggacgcgcggtctgccgatgctggtgttggggccaaacttactgctggcatcgtcggcgctggtgtagacccgctgagttacgttccgctagtaggcgtagccgggaagggtctcaaggtggttaacaaggctttcattgtgggtactcaaagtgctggcctcgctgttgcatctgagggtatccgtacatcaatagctggtggtgaagcacactacgccgatgctgctttaggtggtctcatgttcggtgctgggatgagtgcaatcagtgacgctatcgcggctggtattcgccgttcccgtgggactgaggttgttaatgacttcgctcctatggcacaccgcttggaagcccgtgagaccgctctgaacactggtggtgaagacctgacccgtatgcctagcgagaaccgtgtgttcgaccgtgagcacgctggcgttgagtattcacctctggataccgagccgggtgccgttgtgttgcctcaaggtcaaatcctgagcgacaccaacccactgaaccctcagactcttagcgagttcgaggcggtcaaccctgagcgtgcctctcgtggtatctctttgggtggtttcactgagattggcttgaagactctccgctctgagaacccgactgttcgctccattgcgagtgaccttgtgcgttctccgacagggatggaatcaggctctaatggtaagttcggtgcgacagcttctgacattaaggagcggctccacgcgaacaatcagcgcacctacaatcagctttatgatgctgtgcgtactgcaatgaaagaccctgagttctccacgggcggggccacaatgagccgtaaggaaatccgacaggaaatctacaagcgtgcagccttggcgattgagcggcctgagcttcaagcaaacctgacaaagggtgagcggaacgtgatgaacatcctcaagcagcactttgacctcaaacgtgaaatcatggagaacccatcaatcttcggcaacaccaaggcggtcagtatcttccccggctcacgccacaagggaacctacgtgccaaacgtgtacgaccgagctatcaagattgagatggttaaacgctacggtaatgatggcttgcagcgagcaatcgcggagtcatggttgacgtcttatcgtgctcgtccagaagtcaaggcgagggtcgatgagtacctgatggaacttaacggtctcaaatccgtgcaggaagttacacctgagatggtgcagaagcacgcgatggataaggcgtatggtatttcacacactgaccagttctctgcctcctctgtcattgatgataaacttgagggtctgacgggtatcgagaacaacaacttcttggaagcccgtaacatgttcgacagtgacatggcggtgactctccctgatggtaacacgttctctgtgaatgacctgcgtacctatgacatggctgaaattctgcctgcatacgaccgacgagtagatggtgacgttgcaatcatgggcggttccggtaagactaccaaagacctgaaagatgaaatcatggcactggataagcaggcagagggtaacggcacactgaaaggtgaagtggaagccctcaaggacaccgtgaagattctaactggtcgtgcccgtaggaacccggaaggcgcttggggcacagccctacgctccgtcaatgacctcacgttctttgctaagaacgcctatatgggcgcacagaacgttactgaaatcgctggtatgctggcgaagggtaacgtctctgcgataactcatggcatccctatgattaacgattgggtcaaccgtggtaaaccgcttcgtgcctctgagattaaggagatacacgggatggtgttcggtaaggaactagaccaactaatccgtccgggacgtgaagaccacgtgcgccgactccgtgagtctacggacaccagcgctgcggttgccaatgtggtaggtacgattcgtttcggcactcaagagttggctgcacgctctccttggacaatgctcctaaacggaacatcaaactacattcttgatgctgcccgtcagggtgtgcttggggatgtggctggagcggcccttgctgggaaagcctctaagttcggtaaggcaaactaccttaagtctgcctctatcagccctgagcagtggaatggaatcaagcagttgttcagagaccatgcgactcgtggagaagacggtaagttcaccattcgtgacaaacaggctttcgccaacgacccccgttcaatggacttgtggcgactggctgacaaggtggctgatgagaccatactgcgccctcacaaggtttcctcacaggacgccaaagcgttcggtgccggggtcaagatggtgatgcagtttaagaacttcgtcattaagtcactgaactcccggttcgtccgcagcttctatgaggccactaagaacaaccgtgcgttagaccaagcgttgactcatatcatctccttgggtctagctggtggttactacgtggggcaggcgcacctcaaggccgcaagcctacaggagcacaaacgtaaggagtacctcaagaacgcccttgaccctaagatgattgctcatgcatcaatctctcgtagttcacacttaggctcaccattgagtatctacgacatgttcgctgggatggttggtagcgatacctataagtacacccgctctacggtactacctaaagagtccgagaagcgagaccctaacaaggcagtgacaggtagacaggtggcttcaactatcgcaggtgcaattggtgagcaggttccgggtttaggctttgttggctctgtgggtgcaacagctattaacgccgcttccctgctgtcttctccgaacaaggctaccgctctggagttccgtacaggactcttcaacacctctcgtgagttgattcctaatgacccgctatcgcagcaactcattatgaaaatctacgaggccaacggtatccgcataaaggagacgccgaagaaacaataaaccctcactaaagggagagaggtcacattctggcctcttctctcttaatgctaatttacaaaggaggtcacatggctacaactattaagaccgtgatgacttacccgctggatggctccactacggactttaatattccgttcgagtatctggctcgtaagttcgtccgagtgacccttatcggtgttgaccgaaaggaactcatcttgaatcaagactaccgttttgcgactaagaccacaatctccacaacgagagcattggggccagcggacggttataacatgattgaaatccgtcgattcacctccgctaccgataggctggttgacttcaccgatggttcaatccttcgggcatacgacctgaacatctctcaggttcaaacccttcacgttgctgaggaagcacgtgaccttaccgctgatactattggcgtcaacaatgatgggaatttggatgctcgtggtcgccgtatcgttaacgtagctgacggagttactgatggtgacgccatctctgtaggtcaggttaaacgtatgaaccagaactcgtggcaggctcgcaatgagtccttgcagttccgtaatgaagcagaaggtttccgaaatcaggcctctacgagtcaacaggctgctgctacctctgagtcgcaatcatggaatcattctgagcgctctcggacattcgctgaggcagctcaaggttctgctaactctgcgggacagtcagctaccaatgcgaacaacgctatgcagtctgctggtcaatcagccatagatgctagtaactccgctgctcaggctaaagcctctgaggttaacgcaaaggcctctgaggttaacgctaagcgtgatgctgatgctgtcatggtggcgctgcaatccactggtaacgtacctgtgggtacagtcgccatgattacccacaccaagattcctactggttgggtgcgcgctggtgaggagttcgacgttaatacttacccggcgttagctgaactgttccctagtggtcgtactcctagcttcgatgaccgctatcctattggtaactctactgtgttgactccgggtcagcttattgaccaatccgtgccagcccacagtcacaccttcgacgtgcctgtcaacgtgtcaggtgctacggctgctgctggcgaatatcgcgccagaacgtcacatgaaggagaccacagtcacgggttctcattgcctatccagaacaacactggtgcttatactggtcgtctggttggcggtggcaacaaccctaactacccgcaagacctgcactttaacactggtggcggtggtgctcacagtcatgagttctatgttccttcacacagccacacgttgaatgcatctgggcgtgctgctggctccgtctctccgtcaggtattggaaactcaccgtatgtccgtccgtattccactgtggtcatcttcatcatcaaggccgctcaaggtgttgacgataaggacgcagcaatgcaggtggtgggcactgtggttggacgagtggacgctctggagaactggcagaaaaacttcaagtccgttgttgagaacggttcaccaacctctggaaacggcgggccttgggaacgtactgtgtacaccgcccagactgacttcggtctcaaccgctggcgcacgctgagtggttcatcatggggagaagtagtcaacttacttaatagctccggcgtacttaaaggatatccgcaacgtcttgttgttgacttcacggttgaggtaatcgatagctctgacctaatcaccaaggctggtatatatagtccagtgattatggccagaatcgtgtatcacaacggcaccaactctggtgatatacttcccgctgcaacctcaagctctgcgcacgcgacaaacgggtggaacattaagatggctacacgattgttgtatggtactgatggcggttcaccagtagttgagattggtgccttcaaggatggttctgaccagcagacggcaaacgtgccagttaaagtaggcttacgcctagttcacgtgttctaaggaggacataatgttatcatttgacttcaacaatgaggtcgtcaaggctgcgcctattgttggcacaggggtcgctgatggggcggcccgactcttttggggactatcgttaaacgaatggttctacgttgcagctatcgcctacacagtggttcagattggtgccaaggtagtcgataagatgattgactggaaacgtgcaaataaggagtaacctatgagcgacaagactttaatcaggctgctggagatgcttgacacggagatggcacagcgtatgcttgctgacctccagagcgaggaacgccgaacgccgcagctttacaacgcaatcggcaaactgttagaccgccataagttccaaatcagtaagctgaccccggatgagaacatccttggtggtctcgcggctggtctggaagactacaacaaagtggtcggccctaatggtctgactgacgatgaaacaatcacgctacagtaagtgacatactcaaggttctccacgcggggagcctttatggatgttatttggtgcatctacgtgaaatctgaaaattgatgggaggtgttatgctaaaacttttacgcagcgcactcccttgggtactcgccgggacactctttatgggtggctggcacttagggtcaacccatgagagagcaaactggaaggaggtaatccaaaatgaatacattgcgaaaacaaaagcaaccgcagaaactcaggcagaggtcagtcgggtgtcccgtgagtaccaagaagagattgcagccattgaaggcagcactgataggatgcttaatgacctgcgtagtaataataagcggctgtcagtccgcatcaaaacccttaccggactaccagaagataacggtagatgcgagtttaatggtcgagccgaactacacgagtcagatgctaagcgtattatcggaataacccaagctgctgatgctcacgtaagagcgctccagcgtactattaaggagatgcagaatgagcgacacccaagcaaaccgtaatgcgctaatcatcgcgcagcttaagggtgacttcgtggccttcctgttcgtattgtggaaggctttggctctcccgcagccgactaagtgtcagattgatatggcccggtgtttggctaacggagacaacaagaagtttatcctacaggctttccgtggtatcggtaagtcattcatcacctgtgcgttcgttgtgtggacgttatggcgtgaccctcagttgaagatactgattgtctctgcatccaaagaacgtgcggacgctaactccatcttcatcaagaacatcattgacctgctgcccttcttggctgagctaaagcctcgccccggtcagcgtgactctgtgattagtttcgatgttggccctgccaagcctgaccactctccgtctgtgaagtcggtgggtatcactggtcagttgactggtagccgtgctgatatcatcatagcggatgacgttgagattccgtctaactccgcaactcaaggtgcccgtgagaagctgtggactctggtgcaggaatttgctgcgcttctgaaaccgctgccgacttctcgcgttatctaccttggcacgcctcaaaccgaaatgaccttgtacaaggaactcgaagataaccgtgggtacaccacaatcatctggcctgcgctctatccgcgtagccgtgaggaagacttgtactatggcgaccgtctggccccgatgctccgcgaagagttcaacgatgggttcgagatgctccaaggtcaaccgactgaccccgtgcgcttcgatatggaagacctccgggagcgtgagttggaatacggtaaggctggcttcactttgcagttcatgctcaacccgaacctgagtgatgccgagaagtaccccttacgcctccgtgacgctatcgtgtgcggtctggacttcgagaaagccccaatgcattaccaatggcttccgaaccgtcagaatcgcaatgaagagcttcctaacgttggccttaagggtgatgatattcatagctatcattcgtgcagccagaacactggacagtaccagcaacgcatcctcgtgattgaccctagtggtcgaggtaaggatgagacaggttacgcagtgttgttcaccctgaatggctacatctatctgatggaagctggcgggttccgcgatggttattccgataagacccttgagtccctcgctaagaaagcgaagcagtggaaggttcagacagtggtcttcgagagtaacttcggggatggtatgtttggtaaggtattcagccctgtgctcctgaaacaccatgcagccgctatggaagagattcgcgctcgtggtatgaaggaactacgcatttgtgatacgctggaacctgtgctctctacgcaccgccttgtgattcgtgatgaggtgattcgtgaggactaccagactgcccgtgacgctgatggcaagcatgacgttcgctattcgctgttctaccagttgacccgtatggcccgtgagaagggcgctgtggcacatgatgaccgacttgatgcattagcattgggtgtggagttcttacgctctacgatggaactggacgccgtgaaggtggaggctgaggtgcttgaggcgttcttagaggaacacatggagcacccaatccattcggctggtcatgtagtcacctcgatggttgacggtatggaactctattgggaggatgacgatgtgaatagcaacaggttcattgactggtagttatgcatattgagtgcataaggattcaattagaccacggatggtcactttaagaaattccttaagaatcaacgagtttgcattaaccctcactaaagggagagaggggacttaaagctactatataggttagtcattcaggttatgcaatgaccctttatgcactttaagtctacctctgtgattggtgatattatcattgtataatcaccctaccttagagcaactgaaaggaggtggctcaatgttacgcttgctgattgccctgctgcgtcatagagtcacttggcgatttcttctggtacttgctgctacccttgggtacgcaggtcttactgaccacctcggtcaactggaagtggccttttgctctatactctcttgtggggattaaattgttgttgatgaccgacaagcggctctgagggattcgtaggtatagtttcactacaccaactcatccctgtagagtcaaccctaaaggttatacctaaagatgccctgtagttcgtaatggtcttacagggtcttctggtgtctcctagctatccctttgggatgcgcctgtggtctcacctatggtgtggccttacctaaagtggttgcctaaagagaccctttaagaatttaccacaaaaatctgaatgggtatctcacagttcaagaacccaaagtacccccccatagccctcctaaagccacctaaagccagccctacccctagggtttaaccttcggttaaccttgggtatctcctcaagtggcctataggatagcctaaagtgttgcctaaagtcaatacctaaagtggtggatggcttaaggaggccttaaagagtgtcctagtgtaccagttcgatagtacacctctatgtgtccctatctgttagccattcctaaagtaaaacctaaagcctccacctaaagaccagacctaaagccactacctaaagaccagacctaaagccaccacctaaagagcgacactatcttaaagacaaccagaccgatagacccactgttaacctgtagtgacgccctgtagttctctatggtgtctatcgtcaattcaattaccctcactaaagggaataagggatacttaaagagggaattaaagatttaataaaaagatttaaattaatggttgactttaagtaacccttaaggctattattcatctcgtcaacgggacaacggccttaaagccactccctgagataccggagtcaaccggataagtagacagcctgataagtcatacgaaaaacaggcattgacaacatgaagtaacatgcagtaagatgtaccacataacgtcaaccacaaatcggtgtcttctaggtgacttaagcgcaccacggcacataaagcgaaacaaaatggttgacaacatgaagtaagcacggtacgatgtaccacattgaaacgacagtgaggcgggacactgaaagccgctagggtctaacgaaaccagaccgacacgctctttaacaatctggataatagctcttgagtgtaccgatgtataagcgaataactcaaggtcgcactaaaagcgtggcctttatgatattcacttaacacgaactaacatgaggtaacaccaaatgattttcactaaagagcctgctaacgtcttctatgtactggtgtcagctttccgttcaaaccttgacgatgaggtgaacatgagccgccaccgccacatggtaagcactttacgtgccgcaccgggtctttatggctccgttgagtcaaccgatttgaccgggtgctatcgtgaggcaatcgcaagcgcaccgactgaggaaaaaacggttcgtgtacgctgcaaggacaaggcgcaggcactcaatgttgcacgattagcttgcaatgagtgggagcaagattgcgtactggtatacaaatcacagactcacacggctggtctggtgtacgctaaaggtatcgacgggtataaggctgaacgtctgccgggtagcttccaagaagttcccaaaggcgcaccgctgcaaggctgcttcacgattgatgagttcggtcgccgctggcaagtacagtaagtgatagactcaaggtcgcactgtatgtgtggcctttatgattatcacttagcacgctataggactaacaccatgagtaaactactggcaacatctaaaatagaaggccaatgcaccgtaacactgcgtgagtattaccacgggtcaatgggaagcacctacgttgtgcgatacggtaaacaggtcacacattgggtaaatccgattttagcacaagaggattatcaagcctgtgtgatacaccaagcaacatgtgcgggctggaatgactgacataacaggaggattagcaatggctatacttatcactatcgcactctttgcggtcgctggctggtactctgagtcacgcaaggccgctcgctatcatcgcaagatggttaaactcatggaacacttggacgcagaagggcgaggccgcgaggctatcgaactggtgaaaaacgcatgatgattctctttatcgcactggtttatctgtgcatcgcaatgtactttatgcgagactttaagcgcggtctggcaatccacaaggcaacgctgagttactttaagtggggattcatcccacggtttacggttcgtgaacgcaacggacgctttaaggccaataaggttggtatcttctacatcgcaactcactgattaacttaaggacacactgaaatgactttcaccgaagcacaagccgcattcgaagctatccgcaacctaccgattgagcgcctgaacgaacgccagcccatgctggtggctcttgcggctgctatcgtgaacgatgagactcaagacggcgaactgacgcaggccaaaggcaattggggcttggagtcgatggactactggcagacactggcggagcgtatgcgtgatgttggtttcatcaagctgggcaacgggcactttagcgcagcctacgagcacgcactgctaccgggcaaggttatcaaggtaggctttaagaaagaggactcaggcgctgcctatgctgccttctgccgcatgaatcagggtcgtgagggtatccctacggtgtacgacattcagcgtcatacaggctgctacacggtggtcttggactatctgacaccgcttaacgattggtgctacggtgaaagtgataagattcgccaccacttccacgctgcacgggaaatcattcaggaggattgcttcgacaccgccgaagagtatccgataagcgagggctttattgagacctgcaaggatatccgcaagttcttcctaggcatcgcatcgttcgactgtcacagcggtaacatgatgaaagaccagatgggacgcctgattatcacagacccggtatcattcagcgcagacgacaagttaaagccgggtgagttccactgtgacccggatgaactcatcgctgagattgaggcgctgagagcgcaggaagcgattgaccgctgtaaggcacgcaaggcacgccgtgaccctgacggaacctttcaggtagcacgtaaagcccgcgccaaatggcgcagatagaccacagggctatcgcatggaacgctccgctgtgtgtcgataagtaccttgacaagcgtctacaaggctgatagagtcttatcttacaggtcatcaatcggtggcctgaataggtacgatttattcacaatgaggtaagcaatgaacatcatcgaaactatcgaaaagaatgacttctccgaaatcgaactggctgctatcccgttcaacacactggcagaccactacggcagtgcattggctcgtgaacaactagctttagagcatgagtcttatgagctaggcgagcgccgcttcctcaagatgcttgagcgtcaagcgaaagctggtgagattgcagacaacgcagccgctaagccattactcgctacgcttctccctaagttaaccgcacgtatcgttgagtggctcgaagagtacgcatcgaagaaaggccgcaagcccgtagcctatgcgccactccagttactcaagccggaggcgtccgcgtttatcacactcaaggttatccttgcgtcactgaccagcacgaacatgacaaccattcaggccgctgctggtatgctggggaaagccattgaggacgaggcacgtttcgggcgcatccgtgacctcgaagcgaagcacttcaagaagcacgttgaggaacaactcaacaagcgccacgggcaagtctacaagaaagcctttatgcaggtggttgaggcagacatgattggtcggggtctgctgggaggcgaggcgtggtctagctgggacaaggaaaccacgatgcacgtagggattcgcctgattgaaatgctgattgaatccacgggtctggtggaattacagcgccacaacgcaggtaacgcaggctctgaccatgaggcactgcaactggcccaagagtacgtcgatgtgttagcgaagcgtgcaggagcactggctggcatctctccgatgttccaaccgtgtgtcgtaccgccgaaaccttgggtatcaatcacaggtggcggctattgggctaacggtcgcagacctttggcactcgttcgcactcactctaagaagggactgatgcgctatgaggatgtttacatgccagaagtttacaaggcggtcaacatcgcacagaacaccgcatggaaaatcaacaagaaagttcttgcggttgtcaatgagattgtgaactggaagaactgcccggtcgcagacattccatcgctggagcgccaagagttaccgcccaagccggacgacattgacaccaacgaggcagcactcaaggagtggaagaaagcagccgctggtatctaccgcttggacaaggcacgagtgtctcgccgtatcagcttagagttcatgctggagcaggccaacaagtttgcaaacaagaaagcaatctggttcccttacaacatggactggcgcggtcgtgtgtacgctgtgccgatgttcaacccacaaggcaacgacatgacgaaaggcctgctgaccctcgctaaaggcaagccaatcggtgaggaaggtttctactggctgaaaatccacggtgcgaactgtgcgggtgtcgataaggttcctttccctgagcgcatcgcgttcattgagaagcacgtagacgacattctggcttgcgctaaagacccaatcaataacacttggtgggctgagcaggattcaccattctgtttcctcgcgttctgcttcgagtatgcaggcgttgcgcaccacggtctgagctacaattgctctctgccgctggcgttcgatgggtcttgctccggtatccagcacttctccgcgatgctccgcgatgaggtaggcggtcgtgcggttaacctgctgccaagcgaaaccgtgcaggacatttacggaatcgttgcgcagaaagtaaacgagattctcaaacaggatgcaatcaacggcacacctaacgagatgattaccgtgaccgataaggacaccggggaaatctcagagaagctcaagctgggaacctcaacgctggcgcaacagtggctggcatatggtgtaacccgtagcgtaactaaacgttcggtcatgacgttggcttacggttccaaggagttcggctttcgtcaacaggtactggacgacaccattcagcctgcgattgacagcggaaagggcttgatgttcacccaacctaaccaagcggctggctatatggctaagctgatttgggacgcggtgagcgtgaccgtagtcgcagcggttgaggcgatgaactggctcaagtctgccgccaagctgctggctgctgaggtcaaggacaagaagaccaaggagattctgcgccaccgttgcgcggttcactggactacgccggacggcttcccggtctggcaggaataccgcaagccactccagaaacgtctcgatatgattttcttagggcaattccgtctgcaaccgacgattaataccctcaaggattcaggcattgacgcacacaagcaggagtctggcattgcgcctaactttgttcactcacaggacggtagccacctacgcatgacagtcgtttatgctcacgagaagtatggcattgagtcctttgcgctcatccatgacagctttgggactatcccggcagatgctggtaagctctttaaggctgtgcgtgaaacgatggtcattacctatgagaacaacgatgtgctggcagacttctatgaccagtttgcagaccagctacacgagacccaactggacaagatgcctccgcttccgaagaaaggaaatctgaacctgcaagacattctcaagtctgactttgcctttgcataacaagcacttagcattaaccctcactaacgggagactacttaaggtctcccactttaagacactttaggtactaagagattaaatttatgattaacattaaggctttctttaagaacatctttaagttaaaccgcttgacttccgttaagttctacgcttggatgccaggtagtgatgacctccgcaagactgaatttaagttaggcttagggccgtgcggtaaggtggtcaagaagttggaatgttactcaacgtcctccgggatggtcatcttccagaccactgaggacaacgaaacgaaatccttctactacccgaaaggttcaacgtgtggtcgtatcgaacggacatacagttaaccctcactaacgggagagttagcctcaaggtcatccaagcgggtggcctttgtgattaactttcaatacacatcaacatgaggtaagatactatgcgtaccaactttgagaaatttaccaagcgcgattcagtggtcaatgagcatggcgaacagtggcaagagcgccgtgaccgcatgaagaaacgccacaagcagcagcgcggtaactcacagaaacgggagtggaactaatgatgggacgtatctatagcggcaacctgaacgattacaaagatgcggtagcgcgtctacaggaagaccatgacgtgaccgtgaaggtggagtcgttcagctatgaaaaccccgcgaagatgtgcaggtcatccggtgaggttctccgtgtgttcacacgctccgggcatccggtggcatccagaaccttcgagcatagcgacagcgatgttcaaataaacgcgcagactgcatggctccgtaaggttcacagtgacttgaaacactggaagtaataaccctcactaacaggagaatcctcaaggtcactttaacgagtggcctttgtgattcgaactgattaacttccaaccaatgaggaacaaccttatgaacatcttcaagaccaacccattcaaagctgtgtcattcgtagagtctgccgttaagagagcgctggagacctccggctacctaatggctgatatcaaatatgatggtgtgcgcggtaacatcgtggttgacaacgtggctgaggctgcatggctgtcccgtgtgtccaagttcatcccggcgctggaacatctgaacggtttcgataagcgatggcagcaactactgaacgatgaccgctgcatcttcccggacggcttcatgctggatggtgaactgatggtcaaaggcgtagacttcaacacagggtctggcctgctgcgtaccaagtggctcaagaagaacaacttcatgtttgaccgtggtggtgttgagccgctgaaaggttccaaagtagcctttgagttagaccctaagcgcctgagtgtgcgcctgtatgcggtcatgccgattcacattgctgagtctggcgaggactacgatgtgcagaacctcctgatgccgtatcatgtggaagccatgcgctctcttctggttgaatacttcccggaaatcgagtggcttatcgctgagacctacgaggtctacgatatggattctctgagtgaactgtacgaggccaagcgtgccgaaggtcacgagggtctcattgtgaaagacccgcagggcatctataagcgaggcaagaagtctggctggtggaaatgcaagcctaatgacgaagccgatggtattattcagggtgtcaattggggtacgccgggtcttgcaaacgaaggtaaagttatcgggttctctgtgcttcttgagactggtcgtctggttgacgccaacaacatctctcgcgcattgatggatgagtttaccgcgaatgttaaagaacatggcgaggattactacaacgggtgggcctgccagattggatatatggaggaaacacctgatggctcattgcgacacccctcattcacctgcttccgaggaactgaggacaatccacaagagaaagtataagttagaccgggaggctccttcgggagtcgtttccgcctctggtaaacctttagggtataagcggaaagatggctactggattctctcttacggtggcaaacaaatgctggcacatcgtgtggtctggatgttgacccacggtgaaatcccggaaggtatggtcattgaccacatcaacaggaaccctagtgataaccgtatcgaaaaccttcggtgtgtcactcaagctgttaacttatcgaatgttgacgtccggtctcattgtttaagtggcgagaagtatattgcccttgacaagagaaccggacgttttgccgttcgtatcaagcgcaaatcccacggaacctatggaacccttgaggaagctatagccgtgcgagatgctgccattaaccctcactaacaggagacacaccatgtggcttatcctattcgctatcgtcgcaacgctgggattaatggctgccgacgacaacatttggcctgattgttaaggagacaacatcatgcgtttacatttcaacaaatccaacggtatcttttcggttcgccgtaaagaccgctctaccgtattggcctctgagcgcaacgccaagctgccgctgattggttcggtcgtcccactatcgccccgtgttcacctgctgataactcgtggagagttcattaaggcgatgaacaaagagcgcccacatctggaagccgtggtcacttactggcctcgcatccgtctgttcgttaagtggattaaggaggtgctgtaatgttacaggaatattacgatatgcgtagccgcttcccggttaactctgccgttcgttattcaggagctaaccccttgttcaagggtctgaccggaacagtgcagggctactctcacactggtcgagtaaaggttcgcttcggtattcgtgaggctgaggttcacccatccgtgctaatcccgctacctaaagtcggcccgaacgtggaagcaccgaagacagaagtcaagtctgacgtgacccatccgaaccactacatgctgttcgacaacgtggaagccattgaggttattgcccggtcaatgaccgtcgaggcgttccgtgggtattgcttagggaatattttgaagtaccgcctgcgtgctggtaagaaatccgaactggcaacgatggagaaagaccttaagaaagctgcattctatcaggaactctttgacaaacaccgggggctgtgttatgacgcttcgtgaatgttgcgattggtgtgccgctaagtggaaccaagctattgaggctggtgacgaggccgcagccaacgcctaccaacaattgtacgcgctgtgggagcggcgctttaaggaggctcaaagtggagcaacgtgaacagaaataccttctgacgatagaaggcaacaccgagtcctttgaggttccggtattcgcccggtctctcgaagaggcaacgctacaggctgaacattatgaggacgctgggttcgtagtgactcgcatccgtccagaagtgaagcctaattaccctcactaaagggaacaacccaatcattaaccacaaaggagaaacattatggctggtttcaagaagaaaatctacacctctggtcttggcactgctgagccttatgcttacctgagtaagccggattacggcaacgaagagcgtggcttcggcaaccctcgtggtgtctataaggtagacctgactctttccaataaagacccgcgctgtcaggcgatggtcgatgaaatcgtgaagactcacgaagaggcttatgctgctgccgtggaagagttcgaagctaacccgccgcaggtacagcgtggtaagaaaccactgaaaccttacgagggcgacatgccgttcttcgataacggtgatggtacgactaccttcaagttcaaatgctatgcgtctttccaagacaagaagaccaaagagactaaacacatcaatctggtcgttgtcgatagcaagggcaagaagattcaggacgttccgattatcggtggcggctctaagctgaaagtgaaatactctctggttccttacaagtggaacaccgctgtgggcgcaagcgtcaagctgcaactggaatccgtgatgctggtcgaactggctaccttcggtggtggcagtgaagatgagtgggccgatgaagtcgaagatggcggttacactgcgagtgagtcccgtcagtcccgcgatgagcaggagtggcaggaagacgagcacgaagaaacccctgatgaggatgaggatttttaaaccatgaacctgcgggagaaactggaggcttactctaaagtgtctccctctggttgctgggagtggcagcgagccactgctgggaagggttatggtttaatatccataggtcgccagcgtcaagcatatgcgcacagggtgtcatacttcgtctttaacgggccaatttccgaaggtcttgtagtcagacataagtgtgataacccttgctgcgttaaccctgagcacctagaagttgggactcaggaagacaacatgcaggactgtaagaagcgtggtcgtctatccgcgccaccagtagcgagaggtgtacacaatcacaagaccaagctgaccgatgaacaggtggcctacattgtgaactccaagaagagtagccgcgagttagccaacgagtttggagtctcgccgcaggctataaactggaggaagaaacaatggcaggctacggcgcaa

SEQ ID NO: 2 - DNA, 38786 bp, unidentified; identical to SEQ ID NO: 1
  source 1..38786: mol_type = genomic DNA

SEQ ID NO: 3 - DNA, 43238 bp, unidentified
  source 1..43238: mol_type = genomic DNA
acttcgtatcgtgcgggggaaagtggtgttaagacgtggcaaggtacggtaggttctacgacctcccgtaactacaacctgcaattccgtgattctgcggcactataccctgtatgggacggcttcgatttaggtgcggataccgatatgaacccagaagatgaccgcccaggtgatttcccatattctcaatacccggtgcatatgctccctctaaaccatttaattgacaatctactggttagaggttctttgggggtaggtttaggcatggacgggcagggcctatatgtttcaaatataactgtagaggattgtgcaggttcgggtgcgtatcttcttactcacgaaacggtattcaccaatattgctataatcgacacaaatactaaagattttccagcgaatcagatatacatctccggggcctgctgtgtaaatggccttcgtttggtaggcatacgctccactagtgggcaggggatgacgatagacgctcctaactccactgtaagcggtataaccggtctcgttgacccatcaagaatcaatgtggccaatttagcggaggaaggtctaggaaactcacgaattaatagttttaataacgattctgcggcgctgcggttacggatacataaactgtcaaaaactttagatagtgggtccgtgtattcacacataaacggcggccctggttcgggttccgcgtggacagaagtcacagctatttctgggtctttgccggatgccgtgtcattaaaaataaacaggggcgattatcgtgctgttgagataccggtagcggtgtccgccctaccagacaacgctgtcagggataacggggctatatcactgtatctggaaggcgatagccttaaggcgttagttaagcgggccgatggaagctatacaagattaactttggcataaatagtaaaggccccgtaaggggccttaatttatgataacagaaccacagcagcggtaagtaaagcgaccgcagccaatccgtagcctattaaaaagcatttcgcgccaattgagtatttcatttagcgcccctctctttgtcaacttcctgttgttctagtagccagtcaagttgcgcattagcggcatctctctgttgccgtagtcgtagaacctcctcttccagttccttgatacgtttttgcaatgccggaatcggggctatgatgttcatcattcgaactccataaaatcaccatctaccgactctgtgtaaattagtcctgttctatcatcgtagcacgcaccccaacgattggcggtattatccatcacctcgaattgccctccacctttgtactccgcggagtaataattaccttcatcgaaaacacttatagtagaacttacgcaaaacagtttcatttcttgcctcgtctcttcatgtaattaagcaattcttcctgcaccgatttcttctcgtccgtacgcgcggcaacgacctcatccagcgtgtctttagcgactatgtgatacaggaacactggacgctcgtggccagcctgtttctggcgtacaggacctatacgctcgacaacctgcaaatagtgctccaggttccagccttgcgaaataaacgccagatgatgcccgccgtcctgtaaattcaaaccatggcccgccgatgcagggtgcacgcacaaaatctcgatttcaccacggttccacgcttccatctgcttattgcccttagcgcctttggaaaacgccttcgcctgcgggaatcgcttaaggatgcgctcaagttcgtgcttaaactgataggccaccagcagcggcgcaccctgcaactcctcgacaattgactccagcgcgtccagtttcgtatcgtgcactttctcccagtctttggttgcttctccatccggccctgaaacatacacggcgcctgatgcaatctgcaaacacttcgacgtcttcgccgcagcgttagccgcttcgacttccccgctttccagttcagcgaataacttctcttccatgtcaatgtaggcttgacgcgctttcttaggcaggtcgatttctacaggcacgataataggcgcttcacatccgaaccattcagcagcgtcaatcgtgagactgatatccttcattttctggtgaatctcattatctgcgcccggacgtgcgtgatactcacgcgccatcgccgattttcctttctgtaccgagttaaaccagcggtcggtgaatgcggtgtacgaagaaccaaggcgctcacctgcgtcgataaaccagttctgaccccacaaatcttttaagccgtttggcgatggtgtcccggtaaggttaatgaaacgcttaaccttaccgaacgcaaccttgctaagcgcccttgcccgtttgctcccgcccgaacggctgcggaacgatttaagcttcgtgctttcatcggcaactataacggtaaaaggccagtcgtctttgccgtagtagtcaataagccattcgataacctcgtagttagtgcataccacgttagcgtccgactccagcgccgctatgcggcgcttctccgaaccggttgcatcaatgacacgaaggcacgggaagttccatttctcttgttcagcaggccatgtgccggatgcaacacgtaacggggcaaggattaacacgcggtcctcttccgtaagttgcccgttgcggaacaggcggtttaatgcccacagtacgctgccagtcttcccggcgcccatgcttgcccatatgttgcagcgatggtgtcgcaacatgaacgaagtcattagcttctggtactcgcgcctttgaaacttagacatgattagcctcgtaaaccgcccttgcgaaacccctcggggtaagactgcggattgtttttgttctcgcgctcttgccgcctagtttagcccatcccgggttatccccgctatccgggacaaccgcgacaacgttgggcataataaacccattaccaacccacaggcacgtctttttaacataggcatctcgcgccggaataacttccgggaacataggatgcgcgtcatcttccggcaaatagccgccgtattcgtgggggttgaatacataatcgggcctacgccataaagacgaaagaacgctaaccgggttttcaatcatataaggtgcgttatacttattagccatacgcgccgcaactttacaggtaatcacagcctctacctgaaacgttggattcttcttccgcttcgtttcgaaatgcgccgcgccgctaacggctaaatctgtgcaaggagggaatgcgaaaataatatccggtgcaacgtcgaagtcgaacttattatcgatccacgcgtttatataatggatattttcatgttcaacttttgcgcccaatcgtgcgtaatcaccgtggtcagcgccgtcgtagttgaaacaatagcaagtatggcctgcttccgcccacggctgggccataaggcctgaaccgtcaaacaatgaccagacaatcatttcgccaccagaaccaattccttccgcccgaacgccgtaacgttaccagttacatcttcgataaccagtttaccgttcgactcgacgtacaccgtatcaacggcaacagggcgacgggtcttaacgttgaaaatcatgtcgcccggtacgatgtcacgtgctggtttgcggtcgtattcgtgtttcatttcccaattccttatattgttggtgtgtgattaactataatagctcgctattaggtcgtcaacctgtttaaacgaaccgacgacaaaaacatttgcaccacgtttacgcatccgctcgtgctcacgtaactggtgtgggtctggcttcgtgttttcgtctttcttaacctccacgaaccagatgatgccgtgggggagaattaccagcaggtcgggggcgccggaacgtccttcgtaggaaagtttgcgaacgaggcccccgagggcctctaatcgttcttttgcgtatttctgaatcttgccttccggagtcatgagcgcagaatccaagtaaggaaagccccgcctagaacggcaacgccaacaaacttaaagaacaggccataacagaacatcgcagctattcctgcaccgagtaacagtgcgagcatggttactatcgtccaaaatacaagcattgtcataatatgcacccctcgcgtttcgtatgttcaatcccgcagcgcgggcagattcggcagtcttcttcgtagaaccagtaaattttcattccagcacccacagataaattgcgatgaacatgcccaatatggcaaccagcatgccgtattgtccctcgtgacagtagacaccggcggcgaatcccgccagtaccgcgataatcagtttacttggcataacggtacacctcttccccggcccatgttagtgggaatccttcagcccattcaggtaatgagcatagcaggtcacccatctctttaaccgtgtactccggtgtgtctggtgtttcggcaacgcattcgtcatgtacgtgaagtacaatcgggtagcctgctgcctcgaccgtcaggagtccgttaaccagtaaatcacggcacaacgcctgaatgatgttttcacacaattttccgccgtgtgtatacaaagttgtccactggcgggtagtctggttttcaccctggtacttaatgcgaacgttagttgttacgcgcccatcctcgtcagtttctttagtcacgcttaagcctatgccagggtaggacatgatgcgcccggacggcaactccacgcacaaccaccaacctggcacaaagttaccctgtctgtctttctctttagtacgccagatgcgaatggccctatcgccgttgaggcggactctagcccccgcccaaaactctcgccccggatttctgatcgctgcgattaccgaatccttaaggtcgttccagaatttcactgtttccggatgggcctcacgccacatgcgcttaatagcatcgcaagtacgccagacttctttaggcagcacataagaggcccgttcatcttttttacctggttttggaggtcgcttagcctcctgaatccgcgcccattcataacctcgggcagtggcggcccatatatggtcagggaatgttccccgcattgtctccgtcattttaaagaggtccaacccgtaagttttagccatagtcatgaaagctcctgcgccgccctcgaagccgaggccaagctcgcaacatttcccgacctgacgtaggtccttacgggtggctttaagctcaataggatccataccgaacatcttgcctgcagtggcgcaataaatatcaatccccgcgcggaacgtctcaagggcagacgtctcccccgccatccaggctaacccgcgcccttccacgtttgagtagtcggcaacgagcaacttattgcctttttccgggatgatgcaactgcggacagtcgaggccgttagtttagatacatcaaaacggcgatgcgcccgaccctttaaaagcaaattgatgccgaaggatagcgggttaagcctgcggttccagtcttcgtcggactcccacggctggcgtacaagtttcttttcctcgttagttgggtcgtcatgatagtacccgcgcgacaggttctgcggttggaaccctttacccgcaaatcgtaaagtgcgctttgccccaccgtactggatgcaaccgcggcggcgtccgtctttggaccgtcctagcaggagcggtttgtattttgtcgatgcggtggaggccgcgccgaggcgcatctcaataattgtacgcgcatcgtcaggtaagtcgtcgtcctctagcaaaccttcgagcgttgctttctgcgcgttatagatattgtaggccggtgccagttcccgaaggattggcaggaagtctttgcccgttagtttccccccgaatttcgcgcgggcctcgttttttagctcttgcttgtggcgttcaactgccgctaccgccgcctcagcaagagccgtgtcaacaaagaagcctcggtcgttgattaactggtccagttccagtacacggtcctcaaactcggagttaccccaacgcggcagcttatggaagacctcacgcatcgcagtgatgtcgctaacggcatacttgatgaacagcgcccactcgtcaggatgtgtttcggcggtgtaccggcggattttgtagttctttggcgtaggtttagaaaaacgcttaatcagcgccttgccgcgtttgtctttcgcgttgtccgcggataccccaagcacttcgcacaacgcgtcaagcgaacccggcagcgcgtgacgaaacgcccaaatcatcgtatcaatggtgttacttacaggaatgtcaaagccccagcagttcttcatgatgagtctgtcgaacattgagccgttgtgccacaccatcttaatacgactgttcggcttaaccaggcggcgtaacgcgcgatgcaaatccccgggcatgtcgctgccgtcggtgcaatcccatacctgtacgggctcgtcgtcaaaagcataggtacagataagcacttctgtagttgggtgttcggcgtaagcgtaggagccgactttctttaaatcggcttcggagaatgtttcaaagtccaggtataagtagctcatttctttttccttaaatccagtgccttcgtttcgcttaatcttattttcatacaatcacggcaaatccacacatgccggtatccagctcggtactcatccgcgtaccacggaagcggtttcatatagcgataaacgtgcttacagaaaaacattatttcgacccttaacaaaaaggcccaatgaagggccttagttaaattgattcagatattagcggcgacgacgttcgcggcgcggtgcttcatcttcttcgtcgtcttccaggtcgtcagtgctcgctactacactggagccgccgaagcgtttaccttcacccatatacttgatgccgaggatggtagcgcacaggtttttccaatctttgtgccaccataattctacagatacgtttgcaacacatccgctatatacctgctcgccttcgatttctttcccgtcgatgttgaaatcaggctcggtttgtttttcacctttagccgatgtcataatcaccggttgacggttgtttgtagcgcggaaatacagaccttcttcaaaatcctcaatcggattatctcgcattgccaggtcacggacgcagcatttgtctgcgtggtggtctacaccgtagttggccttcatccatttctcggcagcagctgtcccgattgcctctgttacaacttccagcgcggtgtcgtaaagcgcgtcgatttgagggtggtcaggctgcataagaaccgtaacctgatatttgcccttaatcagtttaccggttttcttgtcctcagaatcagcagcacgttcaaatacgttaacccaagcagtgtttactttacgcagattcagcttaatacccatcttgatttctcgcttttcagtttcgtctactccggggagctgcccggccagtgattagaactataatagctagctagttcgggagtcaacccttccagccagttattttctcaactatctcaatatacttgcttggccatttcatgttgtttatgaatcgaagcgcctgtgtcttccacgatttatcgctcaggatggcttcgatgtcgcgagtttgtgctatacataccgtaatacacattaagataaatgttggtagcgttagtggcagcataagaacccagaaaacaatacacgctacaaatccaccaagcaccctgaacattttacgaatcattctaaatcctcctcggtcacctgattccactcaggacgtttgtcgtctgccgttgcgatacatggtgcacccggcttacgcgtaatgaagtctttcagttcttcttctggtattactttaaccgcttcggcaggcgtcatgagcacttctttagtcagcttgtcgccgtagagttctgccactttctccgcgtctttccacgcacggttgcccatcttaccctcaaccagcttgtaccccggcactttcttaccggaatgcaacgcggcagccatagctttctctaccttgtcgatgtgctggcgcaacagcggcaatttctcatactcagcgacgagttgttcaggtgtcagttccagtgcaaagtcgtcttccagttcttccgccagtacagaattaacggtttttgtacgcgcggcgcactgttctgagaaacggcaccactggcaaccatctaccgacggcctgaagtccgacgctttcaggttcttcttgccgcgggaatacgcatcaagcgctaacagcgcgcgtttctgtgcgaacttagtaaacaattccagaccttcaaccgagatgtcccactccgatgcaccgcccgcgtacggctgaaagatgaccagacgaacagttgttatgttataacgtctcttgagtcggcgataaacaccgagagcataaagcataagctgcttgttttctttcgcttcgacacgatgccgtcctgttttaaggtcgccgataataagcatgtgctcgtcagtgttcgccagtccctgaacggctacaatgtctgcggttccgaaagtctccacgccttcataacccgggtgcaacacctcagtaagatttacgcgcatttccagtttggcgtaagtagatacgtctataatcgctttgcagtagtcagtgtactttcgcacctgctctaccatgtcggccgtaaccagtacagcgcctttcatcggcttaattagcgctttaacagctcctttaccttcttccagtacatacgcaccgatgtcgcgctctaacggcagctcagtgccgcgtatatacgcgtttaaatgatattcagaaaggctatgcattgccgtacctaacactgcagccttgccggaagtgttagggatatctttttcgcatatcagcgacgcagggcatgttaaccatttcttagcaccggatggtgacaataaggcgtgtacatcgttattgccggcgcgttctataagaatcattcttctaccctccaaccgtagttgcaatagttaccgaactcttctttagccatatcttctaattcttcttcagtctcagacccatctaattctatctctacaacttctgaagctccgacccaagctgtttctatagtcactataagtactttactcttcatccgttaaccctcagttaaagcggcccgaaggccgccagatagttattcttcttcgaaatacttgttcttgattgccgtgaggcgttccaggtattcggccaggtcttcgtccctaattgcggcaatcttcatctttttaccggtgaactcttccagcagttcatcagaatcatcgcacgcggcatcgctaggaccttcgttaatcgcatcgtcgatagcctgaatctggtcgcgaagagactggtaatcaatttcttcttccggttctggtaatggttcctctactttagctttacgcggcttgcgtttcgacttctcttcttctgctggtttagtgtcaacgatgtcttcaccttctaccggaagttcttttgctttctcgacttcttccagtgctttagccagttcttttttaactgtttctacccccgttaccgcgacgtcaaccgtcaattcacctactgatttttctatcaactctttgtttaccgcagtgatatcacttttaagttggtacactgtctgtttcgcgctattcgcagcaatcagttcatgggcgactacgaaacgttcaagtaatactaagattttctctaacattatttgttctcctgtttagttggtgataatgactataatcgtactctattatttatgcaagcactttttcgtaaattgcaccgtgtattttattactgtactattactacatatctaactaaggagtaaacatatgcagcaatccgaattaggcgcccgcgtagagcgccgccgtaaagaaatcggcatgggccagactgaacttgccttcaaagctggtgtatcccaaagcctgattacccacctggaaaccggtcgtgtaggtaaggtagactgttttaaaatcttccacattgccgatgctctaggtgttgaccctcgatggctaagctttggtgatacgggggtctgatggccccctttcttttttttttacttacggttaagcatggcagcgcgacaggcgttccatgctctcattgcaagagttgtccggtcctccttcgccattccgttgatttgcacaaagttgcaggcggtggaaaagttcatttcttcaggcactaccggcactggctgctctttgaattcgtcggcataacgaataacccggtcaataagacgctgtatccagcgctctatctggaaattaaactcttcctttgattcaggtaacgcaacaccaacaacacccaacgccttgtccaggtttttagggataaattcgtttcctaccggcacaggctgggtgtgacgatagagcgggatatctcccaccgcctggttttgtttaccccaaatcaaagaagtttctcggccactggcaatgtgacgaagatttcgttcgtcggtgaacacaacggggtcggcacttttctccgcttcgagtgctgccagcacgatacgcgccagcttcaattcgaatatctgcgcttcccacccattagctaaatcggattcaatttgctcaatgaatgccttaacttcttctttggtaatgtcgctcattctaaatccccttctgttgtctttatgttgtcgttaggttcgtacctgttcttcggccgctttcggtcatctatccccgacggcaacctgtaagtattcgttacaacttcaccgttttcattgcagcctaacatcaattcaccctcacgtagcatcttctcgagcaccaggtttgttatactgtggtctccagctctagcgactatttgccgttgtgtgaagcctcgaccagtatcgtcggattgttgtaaatcctcaagagcggacataaccgattcgcgggccgaagcgtctttagaccgtttaacagtatcctttacggaatctttacctttaccgtccaatccttcgttgcgctctttttcctcgtccgtttcgaacggttggaagccccacggcataagtacgagcgctttatgcggttccggcaggtcgaggttaacgatttccccgtatccctcgcccccaacgaattctacagcctggaactctttcggtggcggagcttcgcgaaactgtaccggctccagcaccatgcctattgttttctgctgcataccgtttttgttcttggtgtgcgcaacatttatttgtttctcagtggcccgtacaagcgtaagttcgacgtcgacaccggcgtacagcgccccacttccgcgcgccttactcccacctttcggggtgtggtggacaacgcctacagcgcctttagtaccgtcgcgtacttctttgagcatcgccactacccgccccatcccgtcctggcccgcggagttctcgttaaatttgtctatccagttcccgaaggtctggttaagagtgtcgaaagcgaccatcccgacaggttcactgcctgctagctggcgcattttacgcaccagtttcttcgtatccgcgaactcaccggcatccagaacatgcatatatcgcataccttcatcgccgtatttagcggccaacgcagcgatacgggtgtgcgtgaactccccaccctcaccatcaatatagaaatgatgggctttacgggtgtccgcccccgcgaaccgatacccggcagcactgatatacatcatgccgagtgtgtagaacgatttatacgtcccggattcaccgacgatatcccatatgcaattagacggcatgtacccttcgacgatgaagtccgcttgtggtgctggcggttcttcgtcggccaggtcttcttcgtcgcaagttaccccctcggcccacccaagagctacctctacacggtcaaacggtaagccggtagccgcacaagcgtaagcccacatatcccggccgccgggtttcatcccgcccgtagctaccaggtcggtatcgtggtacagggtaacgttggggcgctcgaagccgtcgcgcggccaacagaacaggaagtcatcctgcttaggttcaccggtggtgtattgggcggcgtgctccggcgtggcgggcatttgcaggccgcggtcggtcatacgtccgccgaattcaaaagcgaactcttcgaacaggtcagttaactcagattgctcaccctccggcactttataatcagaagcaccggtaacgttaatttcaggaattccctccataagcttgctcgccgtaatcatgcgactactttcggacacgatgacctgactcccgacaggcgggcggtacatcggctgagacagggtgaacccggcgctatccacgtcacgccctttaaggaaatgcgtcaacaggccgtaccggatgcggatgatgtcgccaccggttaccggagtgcgcactggcataacgatgcgatagcgtggcgcttcttcggtgtgggatgccgtggtgtagagcatcattgcaaaacgtgactcacgcaccatctcacagtcagttgcgaactctttcggcgtcgcactatccacgtcggcataggccagggatgaagacgtaacagatgcattgcagcgatagaacataccttcggcggcttctttaccagtagagcttaccgcggaggtgcacgcggcggtgatatatccagggtctgtcttggggttccggcgcgaacgtttaagcggctgcataagttcgatgaactcatcccaggttccggaagtcgtggtgtagacgttaatgtcagcgcgttcctcacggcgattgctacgcgaccatgagtatgctaaattgcctgttgacatgtgcttttcctttgtagtgtttaaggccctgacgttcgcgcgttggggcttttcttttattcaaggtcttctggtgtcgcctgagagcctttaacctcagctactgccgaatatttaccggccttacttttaactgctccgtctttaaccagaacctttaacgtgttctcaatgagagaggaactatagtactgaaaataagtccggcgcaagtcaagaacactacacgacccttttttacccgtaagcgcgacaacagacctgaatacccttttctggaaatcagtcactttacattctccctaatccacgcttcaactttctcacggtcaaatgtacccggcaaccggcgacccataatccggacacaacaatccgggaacttgccgtctcttagccagttattcagcgtacggcgggtcaccccgataagctcagctacttcattctgtgtcattctcaaatcctccatctcagttagtgaaacaagtatacacacggcataatgggaaatcaactataaacacacctattgacattttcatttttttcgtgtaagttgtctcttgtaacttcaagttacttaagtttctgtttcaccttgtgagagcttagcggttccgccccaagcggaaacgcgttaaggcttactgaaacccgcaaacttaaaactcagtaacttcccttccttagtatcttaactgccttgggcgagctctattctcatcggcagcatatctcttaagtatctgatttccttgatgtgccgtagattcgcaaccgtaaaaatgtaacgattagtgggcaggttttagacctgccctttagttaccagttaataagaataagggcacaaccctacctgaaggagcacaacggcgacgtaactattctcttgcataccccaacataacaggatgctattcacttatcgaaatgccgaaagattcttagtctacttctcagggattaacgcacttgccttacggcaagcgcttacctgacgagggacgagggaaaagaataaaaatgcacttgtgtaacccaatataatagagtactattattacaggttaacgagaaaagaggatgaatcatgtttaagaagggtcagttggtaatttgtaatatctccatggagtattacgtagtgctgcaccagcacgggttgttgctagcggtcaggaatttaagtaacgggcatatcggatggatgaactcgcagttcgtagcgctgattggaaataactacacggcaaaagattctaaagagtctgcgcctgggtcttaaaggaggttgccgtgttaagtaaatacagaattgttagtgaggagtgggagaccgctttcggtgaaaaacgaattacacctgcggtacaggtccgtgctgggttattttggtgggccactttaacccattgcgcaagcgtgaaacaggcccgttactacattgagcgtgcaggcaacagacgtttggagtctaaagttataggggtgattagttatgagtaagttgggtaagtgtgatgttatattgtaacaactatttaggattttactgagtgacttacgctgctgagtagcagcgtaccatttttgttaatatttttctatttcaccaaatgcaacaatcaacacaactattgtaggagacgcaacaatgattactaaagaacaggaaacatatctgatgaaacttgtccacgatgtagatgatgcttcggcagatatggtccgtgctgtagtgcggggcggcaatgctaagtatgtagcggcgccgctggagcgggatttagcctaccaggaacttatggcctttgttgattcaattacggagcaatctggttgccgtaaagaagatgctcaggggtgcgagcacgattgggccttttacgatacaagtgcgtcgatgatgtgcagaatatgcgggttaaagaaatgaccagtatcttatttatctgggtactgaccgcgggccagatgcaactcgcggcatcggaaacgttttacacattagaggcgtgccggtcagcggcacgcgccgccgagaacgcgcacttcctgtttcagggcgacaagccaaatgattcagaagtacgcgctatctgctcgcctaagcgacttggtaaacaggagaagtgattatgagcctcgcgactgatatactgaaacacgctggtattaaccttgcaccgccttcatcggcggtaacaaagaaggttactcgtgttaaagaaaaggttaagcgtaagccgaagccgcgagtgaagccggttaatgagatgccggatgtgtacccccgcataccaggggtgcatcaaccgaagtattgtgtaggcaaaggtctgtggcgcgcgcactcctacgacggcaagaaagtggtaaacctcggtgagttcagtagccaggccagggcgcatatggcggttaaactgtataagctgtggcgtaaacgcgggcactcagatatcccgcacaagccatccatcagactttatacgttcaggtaatttatatgactactatagcttttgacggtaaaacgatggcatgtgatacatgcgtcacagggaattttaaatattatacagataccaagatttacgagaacgaccgcttcgttatgggtgtatccggggacgccggtgtgggtaggttgttggttgtcgatgccgagatattgacgcctaagtactacgacttcgacttttctgcgctggtgtgggataaagaggataaacatattttcagggtagaattctttaagtcatgggacacgccgttaagctcggttataacaattgcaggtaacgcgacggccgttggctccggtgctccgtatgcactcacagccatgtttatgggtgccaccgctactgctgctgtcgagatagcgaagcgcttcgaccctaatacggatgggggagttataacgcttcaactaggataaatccgaattatctgttactaaaattgtcgtataaatgttaatctccgaagggtaaaccaactttcggagattttttattgtggacgataaatacctttggcttagtgtggcgggcctcgccgggggtgccgtgtctcagattaagaagcgtgaggctatttcgccgtggttgcgattgtgccatctcaccgcttcagcctgttgtgcggtgtacgcatcccccataattataagttactatgaactatcacagtctgagggtcagtacctggttccttttggcgtaggtatgttctggcttaagttatttgaagccgccgactcgtccctcagcaactttaagttaccgtgggggaaataacatgctcaactcaccaatcggcatcatttgcctggtcgcaatcatcatcacctcgctaattaatatttacgcccactggattgaagacggtcttttcgggcgtctgttgtatatggcgtcggcaatgaccgccgccggtgggctagctcatctctttaccggagctattccaccattcataattacaactctcatcgccatactcgcgctgaagtcaattcgtcacatttgcgtgaaaggcgctcgtcagtacaaataccggaggatgtatgtcaaaccgaaacattagcaacaacggcatcaaattcaccgccgcgttcgagggtttccggggaaccgcgtacaaggcaacgaagaacgagaagtaccttactattggcttcgggcattatggtgccgatgtgaaagaaggccagaagattaccgaaggtcagggtcttctgcttctgcacaaggatatggctaaggccgtagctgctgtagacgccgttgcgcatccgtcgctcaatcagtcacagttcgatgcgatgtgcgacctggtgtataacgccggtgccggtgtgattgccgcttctaccggaaccgggcaggccctacgcaaaggtgatgtcgctactctacggaataagttatcgcagttccattatcagaacggcaaatcactcctcggcttgcgtcgtcgcgccgccggtcgtgtcgcgttattcgacggtatgctgtggcaacaggccgaagctattggccgtgcagcaaagtaggttgacacatgggagcattcctaagatactaaacctgctcctgcttattcatccctctaactcctttatcccggtaactgacccctaccgggattttttttatctgtatcccgaaataatagttgactagtagcattaaccttattatgtttagctcatcgacaacgagaacggagtagagaagatgaacttacaaagtgataaagttttttactattgcaaaccgctactggacaccgaagcatttaaagacgcgcaactgatggcacgcattgccgttagtaacctgagcaatagcatcactgcggacgcgttctggttcgctgcaatgcagacacttaaagccgcctatgcaggggagaagaaatgaaagttaaagcaactgaagttacggcgctatcggccttaaaatacgtgacagagagtaaagtatatgacgtggtgtgttgcttcaaacaagatacagggtcaattctcgatatcattgatgatgagggggatagaatttcagttttggttagcaacaccagaaactgcgcacacggcgttaaatgggagattgtagaatgagtgaacaaggtcaagtaagctaaccattacgagtaggacgtaaggtcagtcgcaccccgttcccgacacgcgaggaactgatgaaacgtaacagtttcccggggccggacaagaacaagtatctcaatcgcatgtggggagagcgtaaagaatgactgaccgtgaatacgaaaagatgatgatagaggcggttaacagcggccacgtgatgcacgtcctgaatactaaaatcgctgtagctgagcaaatggttgagtcgctttacgagacacgtcgtgaactgattaaccgcttcaacctgaacaaggggcgcgctatttctgacgatttcgatggggattaccgaaatggagatttttatgagggggccgaataatgcctaaaatcacaatcgcatcactcgagcgccgtattctggtgcttgagtcggagaaacagacgttaggcggtcaactgtcgattaacggtgagtttcaactggaggcgtttaaagagctactggctattaagacgcaagaaccgaacgtgtacgcggggttgttcgttggtgaagacagttcttctctggcttactacaacacggcaaccgcagcttcaaaggatggggcggatgtaataatccccctgtatgacaatgttgtcgggccagaagatgaaagcgcctgaaccagtagttattgatggcgtcctgtggaaaccctactcggttaaccacatcgacgccgacgggaagaagtttagcttctacatttttgcaattagccgtgagcacgccgcatgtgtggtcgacgatatacgagaaacggcgtggcttggtgatgagatagtggggtaagggtatgtttcacgaactaaagatattacctgagcattttgagcctgttttagcaggcgttaagttggcagagctaaggatgaacgaccgtaactttaaagcggacgatatcttgtgcctgcgtgaatacgaaaacggtgaatacaccgggcgtaaagtttaccggcatattatccatgttgctgatgtatccgcgtaccttccgggttacgtgttgctgagtatggaagcgggtatggaagattaattatgttcgacaatattgatgctgcaatcgaagaagcaatatggcgtcgctacaacggcgagcaacagcgacacttctgcctggtgcaacgtggaaacatggttgccgtagtacaggaccgcgataacaaatacccgaatgcgatgcggacaacgaggaatttcttaggatgattaccagcattccgaacctgattaaagaattcggcacgatggctgagacatgccgacaaaccggcatcagcgaaatgacgattgcgaagtaccgcaacgacgtgaactgcgagcgccacgtaatttataacaaccggcttatgacgcacgttaagacaagcccggtgctattcacgcgccggggtatcactaaaactgagcaacgcattgctaaaggggggagaggggaatgagcattttagaattactcagcagcattagttatatagtgttttggttcgcgtttttactggctttctgtactaagccagaaacatttaccaatctgcccggatatatctggtggcctatgtttttcgggctagtggcgtccggtatcgctgatgccctgttgagattcttaaaatgagactgctaatcatacctaacgcttgggctatctccgtagctaacgaccattacggcggcgacggtaaaagagcaccacgacacggcctatacaactgatacaagcccgcttctgtgggcttttctgtacacaggccttcattcccctgtacaatccttaatagacgctcagggggtgtctgatgcgctatgatggtcatacgcatttaagagaggattgcatgaaactcaaattaaaacagcctgacgccgaagtagtggccgccgcgcacgaagaagcggttaacgcaaaccgtcgccgtaaacgtccgcgtggtaaacaaagcctgtatcagtcatccagacattccgccgaactatgggacccggactattgcgacgaattaatccggttcttcgaccgcacgtcatgggaactcgtacccacgtctaaaggcgacgaacgcccgcttatccaggacaaaccgccgtcgctggcccgcttcgcattacacatcggtgtaaccatcccgattatcaagctatggctccgagaggttcctgcattcgccgaagcatatgagacggcacaagcactggaagaggcgtatttcactgagaccggtgccgccgggatatctgctacgttcgccgcagcaaaacttgggcttaataaaactgttgtcgaagaaacgcgtgacgaaccaattagcgaagtaactattaaggtggtgtccggtgaacgttgatatcacggcgacagaaccgcaaggcgcgttccttaatctgcactgtaaattcccggccttcgtagctggcttcggcacaggtaagtccgaggtcatgtgcaactcggccctgcttgatagtatggaaggtggtagcgattcccttatagctatgtacgagccaacctatgacctggttcgccttatcctcgccccgcgcatggaagagaagttatctgattggggtattcgctataagtacaataaatccgacaacataatctatacgtcatcagggcaattcggggattttgttctgcgtactttggataacccagcgcgaatcgtcggttacgaatctttccgtgcaaaaatcgacgaattggacacgttaaataaagaccacgctgagcacgcctggaacaaagttatcgcccgtaaccgtcagttgccgcgtacctacagaccgattacgccgaaaccagccaatacggtatcggtatttacgacaccagaaggtttccgttttgtacatgaccgatgggtcgtaaaaaagaaccaaggttacgaaatgattcaggcccagactacctctaatccattccttccggaggattatgtacagtcattacgggatacgtatccaggccagttaattgatgcttacatcaacggtgaattcgttaacctgacatccggtagtgtttattacgcttacgacaggcgtaagaacagcagtcgtgagactatccaaccgggcgaaacgctgtacatcggccaggacttcaacgtcgggcatatggccgctacggtatatgttcaacgagactacgtctggcacgcggtggctgagttagtagatatgttcgacactccggatgtggtcagggaaattacagaacgatggaaacgacacgggcatcacatcgtcgtgtacccggacgccagcggcaagaaccgtaaatcgactgatgccagtacgtcagatattgcccaattacagaacgcgggtttcgagatacgtgcgaaatcagttaaccctgcggttaaagaccgtgtagcatcggtgaataaagcgctggagtctggtagattaatggtcaacgagcaggcttgcccggttacagcacgttgcctggagcaacaggcttacgataaaaacggtataccagataagacaagcggcaacgaccaccagaacgacgcgacaggataccctatcgcctacgaaatgccgttggttaaacctgtttcccatatcccggttacttttgcactttaagaggattattaaatgttaacagcaaacggccagggttctggcgtaaaaaccaagcactgtgaatgggtgcgctacgcgccgaaatggaaaaaggtacgccacgcgctcgccggtgaacttgttagttatctgcgcaacgtaggactgaacgaaccagacaaagcatacggcgaagcgcgtcaggcagaatacgaggcgggcggcatcgtatacaacttcacccggcgcacgctgtcagggatggttggcagcgtcatgcgcaaagaacctgaaataaatattccgaaggagctggaatacttgcttaaaaatgcagatgggtctggtgtaggcctaatacagcatgcgcaggacacgctcatggagattgactcagtaggtcgtggtggtcttcttgttgacgccccagaaaccggcgcagctacggccgccgaacaaaacgcaggtttgcttaaccctacaatcgctttctacactaccgagaatatcgttaactggcgactcacgcgcgtaggttctgtaaaccgggtaactatggttgtgctgcgcgagacatgggagtacaacgaaccgggaaacgagtttgaaactaaatacggcgaacagtatcgcgttctggacatcgatagcgacggcaactaccgtcaacgtctttttcgtttcgatgccgaaggtggcgcgcaggaagacgtcgtggagatttacccggacttaggagaatcattacgaggggtaattccgtttacctttattggggcgaccaataacgacgccaccattgacgacgctccgctactaccactcgcggaacttaacatcgggcactacaggaacagcgcggacaacgaagaatccagcttcgttgttggacagccgaccctgtttatctacccaggcgagaacttaacacctcaatcattcaaggaagcgaaccctaacggtatcaaatttggtagtcggtgtgggcataacctgggatatggcggtagcgctcagcttatccaggcaggcgagaacaaccttgcccgccagaatatgctggacaaagaacagcaagctatccagattggcgcgcagcttatcactccgactcagcaaatcaccgcagaatcggcgcgcatccaacgcggcgcggatacgtctgtcatggcaacaatcgcgcgtaacgtaagccaggcgtataccgatgccttacgttgggtagctgttatgctgggtaaacccgaagatactgaagtcgagttccggcttaacatggatttcttcctgcaacctatgacagcacaggatagggccgcgtggatggcagacattaatgccggattgctgcccgctacggcgtactatgccgcgttgcgtaaagctggtgtcaccgactggacggacgcagatattaaggacgcggttgcggaccaatcattaccggtcgctactgaagttcagggggaaatccctcaatcggcgcaacaacaagagcagtgaacaaaaggccccctttcggggccttagttttaacgtatatttgccgcgcatttgcgaacaacacgcggtattgcctctttaagctcatcccacgagttgactgcttccatatccaacaacgtcaccatagacttttgaatctcgctccgcaattgattagtttttgcgtcctgggagtcccatgtatcaaaacctgtgacttcacgaatatctatagactccttgattgctttcgctacctgtatgaagtgaccggagtgaccgattctcccctccgtgcgctcaatcattcgactaacggcagcgttaagctcgacaaagctcaccgcaccaatgttacgcagctctacctgccgttgcgtaataaatcggtctataaccaggaagtgaaaatacgcactatactgttctgcaatgtacacggccaggtgcaggcacaaccacgtcttcccgttcctccctcttgatgtttttaacggaacaatacctgtctcctcctcacacacacgcataaagtttttgcagtcatcggtttttagtatctgcgcaagatttcttggttggagtccgttatcgaacctccatctgttcatctggatggttaatgttgaggcatctatatactcatcacgtgtaaattccacctcgccatacggaaattctaaagtctgcacattcatctgtttttctcctcttaattgtatcgtttaaattatattcctaatttttgtggttatgcaacctttaactaccaaaatgaaagcgaataagattatttctcgcagactaaatggccttaattatgcaccctgatgagttgagtgtataatcaccacaccaaaggcgggagggtatatgagtcttttagagtcgattatcagtcaccaaatatggctgcaacgtaacgccagtagcgaagtaaaagacctggcaccatttatctcgcagatgcgcgatgaggttaaacgacaggtactgctgttcggtgacgacagcaggacggcagcgagactcaccgttatgctgcgagaactcgagcaagcactgaacgggattacgtcagaatggtacgagaaactactggaggatgcccgcgagctttccgactacgaagttaactggaacgtaaaaacactgtcgaccaacgttaacgctaactttgtaacacccgcggcggagcaagtgtgggccgctgcgactttcgcgcctcttgaattaagcgaaaagccggttgattttgtttcattaattagcgggtggcggcaaaccgaggtgaatcgcctggttatgggcgttaagtctggattcgtacaagggatgaccacacggcagatagttaataacgttgttgggcccggtggtctggtcgatatttccgcgcgtaacgccgcaaccgttatccggacagcgttggcgcacgtatcgaacgaagcacggcaacaggtttacgctcagaacaacgacatcatcgcgaagtacgagtggatatcaacccttgattcccgtacttcggctgtttgccggtcgcgggattctatgcagtacaaaataggcaaaggaccgctgcctcctgcccatccgaattgtttacttggggacactgtagtatctacctgtagccgtgtctcgaacgtttttaaacggtcttacaaaggcgtcatggtctatatcactacaaagtccggtcgcaccctgtctattaccccaaatcaccaggtaatgaccgaccgtggttgggcggctgcgggcgaactcaatctcggcagcaagctcgtatgcggtgatagtcgggctgtttctctccatcataaggaagataacgtcgtagcccagtttgccgatgtgtttagtgcggctaatgtagcggtcaatcctgcggcggtcagtacaagcccaacctcccccgaagacttccacggcgacggaaccgacggcgaagtcgagattgtacttgtcgacagcctttcctgggataaagtcaagttcggactgaataagcaaatcgttaacaaggagctcccagtggctgccagcgttgatgaccccctccctggctttggccctgcgaagcagttcggcatggtcggcctttctccctcgggcggcttcatgcgctgcggcggagagagcgaggcgctcctcgggggtagctttagccattctgataaacatagcgtcgctgcctccgcgaatagtaacgccgttctctctgagaacgcgtatgactgggctacacgaaacgccgataatctttccgatttcaactggtccgacgccgtcggtgtagagcttgatgatgttgtcgatttggtctttagtgaagctgatttttgcgggcatgtttataacctcgaaaatgaacagaactggtatcttgcaaatggaattatagtacataactgtcgctcaacaaccgcgcctgtaataagtcccgaatttgacttccttgataagggcgcgaaacgggcggccaggggtgcagatggaggtcagcaggtaagcgccgacacgacctactacgaattcctcaagcagcaaccggcgtggtttcaggacgaagccctcggccctgtgcggggtaagatttttcgtaatagtgggataaccccggaagaatttcgtgtaatatcggtagacggattcgggcgtccgttaactcttaaagagatggcggaactcgataagcgtgttgccgattatctgaaagaggaatagagatgggctttttcaaagttaaagatgtgccttcacgacgtgtagttcagtactcccgtgtgtctggtgctggtgaaggtgtcgtgtatatcaaagatgaatcggttctaggtgaatcggtggacgaaatgccgtttgcggataagaccggactggccacaatctcggacggcatcctgtacgaagtaccttatctggacggggccggtgatgtgtacttcgatgtgcaaccggcggatgtgcaactgaaggatggttccgcaaaattaaccgttgtcgtgaaaggtggcaaagcgccctacgatttgcaatggtttaaagatggtaaagaggtaatcaacgttccttacgttgaaggagaactaaccgttaaagaccccggagaatatttcgtcaaggcagtagacgccgacggtgtgtcggtggttagcaaagcagctaaggtctcagaacctaagtaacgaaaggccccttgaaggggccttaattttgccctcaattattccagttattccttgtcaatgtaaattaaggaatactttattcagaaagtagatttctattattttaaatgctacgctaactacgctttcagcgtaccgcggtgcgggtcgcgtgaagcgggcgtctcaacgccctagcgtaaccgcgaacgcattcggaagggccaagctatattgctttcagtatttagctaagattcgccgtataaggtttacagatataccagtttaacgcccgacttacgtcgggcttagcgtataaagaaaggggatactatgaattggaaagtaaccgcagtagccacggcagtcggtatcctatccttgtggtcatatggccagtacaactacagaagcgggtgggtggaaggtcgggcgaatcttgtttcgcagcaacaacagaaagcacaggccgagctagcgaagaaaacacaacggcagaagcagaacgatactaaggctgccgccgccgaatcggaaggtaaagaaaatgctgagtctatcacccgtgaagttatcaaatacgttacccgtcctggccgtactgtctgtgagtttcctcctgaacgggtgtcaatcaaacgacgcgccgccgagaatgctaattccatccccggatacgacgttgatgcagtcaccgtgcaaaatggtgctaccgagtagtgatgccgatgaagacctggtcgtcgatgtgcagaacgcggagtgcatacggcaattgcgtctgaaagtattccggttgcaggagtacataaggaatattctggaatagttgccttagtaagtggaataatttattcttgttacagaaacaccgggtggcccggtgtcctgaagtccaggggacatattgactatgaatcgttttttacgttacccgcttcatgaagaagccggggtggaagataaaccgggcgcaggtgacgcgccaaaaatgtacaccgccgaagaagtgcaggccctgattgagaaagaagtggcggggcttaaggcaaaccaggaagcgctccttagcgagaaaaaggaagctgcacgccgtgctaaagaagccgaagaagaacggcagcgtgcacaccaggaagccttgaaggccgctggtaaaatggacgaatttgaaaagacgattcgtagtcagtatgaacctgtactgaaagagaaagaagagcgttacgcgtctttagccgcgcgaattcttggtagcgaacgtaaggctgttttgggttctttcgctggtgattttatcaccccagaagcggtagaaatcctggcacctttcgttaagaccgagttcgacggtgaagacgtagtgactaaattcatgggtgcggatggcaatgttgttactaccgaccctgagcagttccgcaaatacctgcgcgaacataaagctttttcacatttgattaaagcaaatgcagcttccggtggcggggcttccgggaataaaggcggcggggccgcaccagcgtttaaagatatgagtgaaagtgagcggttggctctctataaatctaaccctgccgaatttgaacggcaacttaaagccctgaggaaataaataatggcaattaccactattggcgatatcgtaaccggcaacatccctgtactggcgtcttatatgaccgaagacccggtagagaaaaccgcgttttttgactccggtatcctcacccctaccccgtatgcggctgagattgctaacggtccgtcaaacattgctaacctgccgttctggaaagctatcgatacctctatcgaacctaactattcgaacgatgtgtatcaggatatcgctactccgcgcgctattcagactggtgaaatgatggcccgcgttgcatatctgaacgaaggtttcggtcaggctgaccttactgtcgaactgactagccagaatccattgcagtccgtagcctcacgcctggataacttctggcagcgtcaggcgcagcgccgtcttatcgcaacagcactcggcctgtacaacgacaacgtggccgctaccgacgaataccatgagcagaacgacatggtagtagacgtgtcagctactcttggctttgacgcaggcgcatttattgatgctacccagactatgggcgacgcattgatgggcaacggtggtgaggttctcggtgctattgcaatgcacagcttcgtatatgctcaggcccgtaaagcccagcttatcgatttcattcgtgatgctgagaacaacaccatgttcgctacctaccagggctatcgcgtcatcgttgatgacagcatgaccgtagtaggccagggtgcacagcgcaagttcatctccatcatcttcggtagaggcgctattggttacggcgaaggttcccctacaaatccgctggcgtatgagcgcgaagaatcacgcggtaacggcggtggtgtagaaacgctgtggactcgtaagacttggttgctgcatccgtttggttacagattcaccagcgcagtaatcaccggcaacggcaccgagactatctcacgttccgctagctggcaggacctggctaacgcaaccaactggaatcgtgtagtagaccgtaagcacgtaccgattgctttcctggtaactggcgtcggtgcttaatcgtagggtatactcttgagggacttcggtccctcttttcatttatacggaggcaatataatgtcaaagacaggtaaagggctaccccgcagcctggttaacgcggaactagatattcccgctgcaaccacaacggcaatcggaggggttaaaaagtccgcaacggtagcggcacctccggcgattagtgccggcagcggtgcagccgccgcggcggctcctacgaaagaagagttcgatgcactggttaccaactacaataagttgcgtacagatgtgacttcattgcgcactgccgttactaatctgctaaccgctcttaaaaacgctggtactgtatcctgaggaggataaaaatggttgatgtaattaaacgtcgtattgttggtgtatctgatgatagtccgcaggatgggcaggttgagattgatatggaaaacgtaatgccgttgcgtttctctaccggtctcaatgacactaccgcggtaaccgctggtcaggctatcacactgaccgtggaactcgccgacggcatggaccctaaaaccgttcaatggtataaggataataacgctatcgctggtgcaaccgctttgacttacactaaggccaactccgcagcggcggattccggtacttataaagtcgtagcgcacgacggttacggtaatatcatttcagatagtacggtagttaccgtaagttaaatacacgcggcctccgggccgctttaaggattagacatgacagataattacgtagtacgggaacaatacaaaggtgtggtcgaggttgacgggcagttagtcccgatgcgcgaagaagcgaacccggaagcattaatcgaaaatcagccagtggccgaagaaccgcattacaacggcggcggagaacctaagcagcgtcgtcgccgcaaaagcgtagaggaataatttatgccgcttatcgtggaaaccggtcaaggcatcccgaatgctgactcttacgtcagcctggaagacgcgaggtccatggcctataagtacggtcttgagctgccagaagatgatgctaaggccgaagcggcgctacgcaacggcgcggtatacgttgggctttttgaatctcagatgtgcggccgtcgtgtatccgcaaaccaggcactggcgtacccccgaactggcgttaccctgcatgggttccctcagccgtctaacgttatcccgccgttagttgttcaggcgcaggtaatggccgcggtcgaatacggcgctggcaccgacgttcgcgggtccactgacgggcgcgaggtgcagaccgaacgagttgagggcgcggtgaccgtgtcctactttaagaacggctattcgggcgggaccgtgagtatcacggcagccgatgatgcgctacgccctctcttatgtggaagtaataatgcttactcctttaatgtttttaggggataaacatggctaagactaaatcggaaatattcgctttgattggggcaagcttccctgataaccagtccggcctaattacgcctgaaaagttgcgagaagttaccactcagatggcggactctatgttgtacggagctaaagaagtagaggtacttcgcgcgtcgtctacagatatccaggcgccatctaccactggtacagcgttaacagtagcttttggtggcgctcagaaaacaagcgccgacccggtaatgattaatgcctccggggcggttacgttcaacgtcgccggcaactacgctatccgtgttaagttacaggccggtcgcaccggggcgagcggaacatccatccttctgtcgcgtgttcttctaggtggtgcgcaattcgggtcaccagcagcgactaaactggcaagcgcagacgccactattcctattgaatcccgtgttgttgttaatgctaccgccgggcagacttttacggtagagattatgcgcgacgccgccggttctaactttggtggactgtacccacaagcggcaacggttacttcctggggtgtagctccatccgcattgctggtcatctcaagactggagggtgtgtaatgggcaccgcttttagtaaacggatgcaaggggtaggtactcgcctactatcaaaatacggcagcacggtaactttggtgcgcaaaggccagaaaacatgggaccctgttttaggggagtacgtgtgggggcctgacgtcgtactccctcttaaagcggttcctgtacccgttaatgccggtcttgtaaacgggaccaccattcaggccggggacatgatggttaaagcggattacagcgtagtgccaaagatggatgacaaggttcggtttagcggtgaacaatggtctgtagttgctattgagaagaagatggttaacgatgacgttgtggcatactttattcaggttagaaaatgagttttgcgcttgatgtgtccaagttcgtggaaaaggctaagaagaatcctgaaaaggtaattcgtcaggtttctataaagttgttttctgcgattatcaaagcgagtcccgtagataccgggcgatttcgtatgaactggatggcttcaggaagcacccccgccgatggaactacggacgctactgataaatccggcaacacagcaaccggtaaagttacaagtttcgttctaaatgctgctgactggcacaccttcacccttactaacaatttgccgtatgcacaacgtctggagtacggttggtctcaacaggccccacaaggtatggtcaggactaacgtcagcaggttccagcaactactaaatgaagaagcctctaaggtgaaataatggcaacatattttgaggacttaacgaaagcattcgacacggcgctggtgacgttcggcatggacaacgacatcaaggtcgcattagagaacatcgacgcgccgacgtctactgatatgccgtatcttgcaagttatatgctgttgtctgatacggagcaagccgacttgttctggactgaacaacgggcaggtgtttatcaggtagacattaacgtcgggtcggccttgggtagcgctcctattaaccgattagctgataagctaaacgtcgccttcgccgccggtaactgttttagtcgtaacgaaatctgtgctgaggtacaatcagtaagcctcggtcctcttattgttgagaatggatgggcgaagaggcctctctcaattaatttcatagcatttacagcgaggattagataatggcgttacaaccatataagggcgcgatgaccgcgcagttttacgttcttgagacgacgccgggggtaacacccgctaacccggtatggcaaccactgcgcaacactgggggtattcccgccgtaacccgcgacgccctcatctctaatgaactggacggcagccgtgaaacatcatctatccgcaccggtaaccgtcaggtaactggtgaatatgctattgaacttagcgcggaaagccaggatgagttgcttgctggcgcaatgaccagttcctgggtagcaggttccactaaatctggaatcagtattaccgtagacccagtagcgaaaactttcacacgtgcaactggcagctttgtgaccgacggcgttgacgttggcgacctggtgcaatttgatggcctgtcgggtaataatggcaaagctttcctcgttaccgcggtaaccgctacggttgtaaccggcgcaggtatccaacatactttaacggcagaatccgacgtccaggccgatttgcgtatcgcagataaactggaaaccggtaacttgtgtaaaacctattcaatcctgacatggttgaaaggtaaatgcggaaatcctgattcgtacatcataactcgtggtgttgaatttaccgggttcactattgagcaggctgttaacgcaatggttaccggttctttcccgttcatcggcctgaatcaggaaatcctgcaagcgccgccgagcggttctgacttcacgaccaattttagcgcccgcccgttcgcatcggtcgacgtatcagcctacgacggtgccgcgccgcttaaacttatcgacacgttcaccatcaccaacgacaacggcgcgtccgcacagtttgagttaggtaataacagcgtggcatttgttgagcgcggtcgcgccaccaacaccttctcgctggccggtaaactgtacgacatgacgttattgaataaattcctggatgaaacagaaatggaggtatcttcggttctcgacggcccggatggcgcaatgagtttcacgcttaaacgcgcttcattgacgtctgcaactccggaaatcggtggccctgagtctatcaccctttcccttgaggggcaggcaaccggcaaccagttccagtcttcaattgttatccagcgcattaaatacgcctaagaaaaaggccccgaaaggggccttagtttcattcttcaagattactcaggtatagccggtatttatccagactctccatcgcctcacgtaagtcggtatcggcgtctttatgcccccgtaaaccggaacacaacagcttcttcaaagcgtgctgtagtgcagggtcacgtacgtcaaaggccctaagaacgtcatacacatcacaggtgatactatcgccatctgagtttgtcattgtacggttgtatttatttgtcataaatcgaggtcctcccctaaaataacagccgcggaaacagcgacacagcctgcatacagaatgcgagcttctaacgcggtaatgttggtaataccctcgtatatagtaacgaacacagcagtgtccataacggtaaacatcagtaccaatcccacggcgaaagcgatagtcactaaccccgcaacaattaacatcggcattaatccgaaaatccacaagattctattcaccccaacatctccggtgaaatagttaaacgtgcgacctcaccatattcggcactataagtaattacatttgcactacgccctgacatccatccgccgcgggaggcgtaggcgtctttagccgccagggtgcggtgttgctcgacaatcatgttacggctttctacaatcttctggtgatgcaggtgcccaacgtgtgcatagctgtaaacactctcgccgaacgctttgcggaatttagcaatcataaccggttcgatagcgtcaaatctggctttatggccgtggtgaaagaacagtgtcgttttgccgtgttgcaccatcttgtacacatccggagatgtatcgacaaatacacgcggttcgttgtcgtacagtgtgttgaacatctcagccaaccaaatcatgccggattcgtcgtggtttccttgcactataagaaggcgcactgatttatgcttaacccgcgccatgttaaccacacgccgaaccatacgaatcatgtagcgcaccagtttctggtagcgtgtatctgcatctaacacatggccactagccggtgtaacggcgtcaaggctatcgaagtgggcaaaatcccctaagagattaataacacccacaccggcatctggcgctttctgaaacgcggcgtcgaaccatttagagaaaaggtcttccgcaatcttcatgtcccagtcatcaccgctctcgtccgcccaagccaacatgcctaaatgaaagtcagaaaccgtgtaaagatttagcaacttttcgttcagtcgctttttagggggagggacagcagaaaccggcgtaatatcagacttcatcccgtcgataaccgcgtgcattaattctacctgacgttcaacatcggtatctgttttcacccattgaagttttgtgttaccgaattcatctaccagcgacgacgtacctttaattttatacccatctggcacaagatggcttacgtcgcgcccgtgcccaacgcctttctttgccaggcgcgcggaacgaagctgaacattacggcgtgacatattgtactttttagctatttctgtggggccgaggcccgcatttagctcttgctgcaactgttcatctgttattttgcgagtgaccatgcttatttcctgattgtttaagacaaatctgattctaataatgtaattttaatttagccagattatttactgttaacgtttgaattgcaggcccagatgagcgcgcctacccacggcagcagaatccatcccagtaaaagattgcatacgaagattcctagtttagccttgtggttgcgcagtaacgctaccaggaacgggataaaataaataaacgcgataacaccgatagaaattcccatgttcttagcctcatattgtttggttgacgtaaggataataggccactattaacggttatgcaagctattttgttattcttatttagcgtctacggtcgcaccggaaaagcgggtggttcccgcctggcgcaattagccaaccagtaaccgactaaccaaagggtattactatgaaacttagcgatttttactacgaagcagaagccgagaaaggcacacgaatgccaattcctttaaaagatggcactgattcaggggaatggttgaatgtggtttcaccggaggccgatgccgccgttaaggctatgcgggcattcacggtggcataccgcgccgcgttaggtaagttgaaaccgcttcgtgataaatgcgaggaggcgaaagacttttccgaatataacatgaagatggaagacgccgccgccgacctaaaccggcaattagcaagtgaacttgtaaacggctggagtcttgatgacgagtttactaaggaaaaccttaaaacccttcttactcaatacaagcgcctggccgagcatgtagtcgtattccaccacgaacagttgcggcaattgcaggaaaagtagacgcgttgtttcagttcgcccgttggaacttcataacccgccacgaaaggcgcaagtttgacagtatagccgacgggcataaagccgcgcttatcgctatgggggtaatcgaagacgcggaagaagcaacgctggacaccgggccggaatgcccccctgaactactcaccacttttgaaaagtatcgtgatgttaaattcacccgccgtgtggatgatgacggggtaaagctatacccaagagaacaacttagttggtcggatttagtggcgtatagctctatttcaggtcagaatatagggatgtttgaatctgaaattatcatgggcttagacgccatttttgagggtagaaacgatggctgatgtagctagcttagtagtaaaagtaaccgaacaaggcgcgaaagccacatcagaccgtcttgataacctttctaaatccgcgaaagttgctggagctgcggtaactggcctggctgccgttgtggccgctacagcgtataaagccgcacaggaacttgtcgagtcacaacggcaactcgacaagatgtccgccagcctgaaaacactcacgggtagtactcaaggcgcaaagcaggccctgagtatcttacaggactttgcccgcgacacaccttacggccttgaacaagctgtggaagggttccgtaaactggtagctctgggcctcactccatcagaagaagcgctccgctcatacggcaataccgcgtcggcaatgggtaaagaccttagtcagatgattgaagcggtagcagatgcgagcacatttgaattcgaacgtctgaaagaattcggcatcaaggccaagcagaaccaaagcgatattgaatttaccttccaggggacgaccactgtagttaagaaaaacgccgccgatattgagcaatatcttctcaacataggcaacgttaacttcgccggggcgatggccgaccaggctaatacactgaacggagctatcgctagtgcggaggactcatggtcccagttgaagatgactctcgctactagtctggatgtagggtcgctagcggaacctttacgctatatcgacgacctgatacaggagataaacgcgcaagtcgcatccggcgaattcgtagctgagatgcggatgtggggggacgtggcgtccgaagttggcggcgcgatagaggcgtctttcgatgcagccttcgggatggtaggtgacgcgctaaacgctttgaactccgcatggacatacaccagtgaaagcattactggaagcggggaggaaaccgcctcgacgatagccgagtcagcggcggatgcgctggacttcatcgcccaggaattcacggcaatggagcggttttttgaagacatggttaaaggcgctcaggatgcgggtcgccttgtaaaggccgcgttaacccccggtgagtcggtagccgaggccaagaaccttaacttccagttagcgcttgctatggatacccaaaggggcgtggctgacctaacacgtaaaagtttccgtgaacaggttgaagctcaggaagacctaattgcgttaaagcgtgctgcttacgacattgataaagaagcggctaaggctgaaggtctgggtaagtttaaggtatcaggcgagggcggagactctaccgacgattccgcggccaaagctgccaagaaagccgccgacgcattcgaacgccagaagaaagccgcggaggatttctattatcagtcggttcaccttaacgacgacgtattccagaagatacaagctaaccaagaagagcaacttactaagttacaggagttctacagcaaccgcctccttagcgaccaacagtacgaaactgctaaaacgcagattatgctcgaggcggatacggcccgccagactgaattagacaaacgcgagaaagagcgcctggaaaaacagttctccgcggatgcgtatgttgctcagatgcaggcccttgctgaaggagaatttgcggaattagaccggcagtacgaggtcaagctacaaaaacttaacgattttcatgctcagggtttaattgccgaagaaacataccagcaaacgttaagcgctatggatgagtcctactcccttgaccgggcgaaggcaaccggtgcagctttcggcaatatggcaagcaacatcgggtcagcactgggggaagcgtccaccgcgtacaaggcatttgcaatcgcacaggcgacaatcgccacgtacacgtcagccgtagaagcgtataaatcaacagcagccataccggtagtcggcccatatctggcccctgttgccgcggcggcggcggtagctgctggtcttgccaacgtaggtaaaatacggtcggcgcgcgagcagggcggcaacctggccgcaggccagatgtctactattgccgaacgcggtaaaccagaagtgattatgcccgccagcgcctcccgtgtccgaacggcggagcagatgcgacagattatgggggagagcggcactaaatccggcggagataatgttactattgtgaacaacaccactggaagaattgattcggcaacaacggaacgtgacgacgaaggtcgtttacgtattataatcagtgaaaccgtaagttcagcgttgcaggatagtaacagcgccatttctaagtcacgtcgcgctacacgcggccaaccaggatattgatatgagcgattaccatttcccggcctctttgaggcctatagtatctaaaggctactcgatgacccgcggtaacaacgtgtggcgggtagacctggccggtggcggagttcgacagggacgagacacctattttgacgtgtttccaattaacgttaccctggtcgtatcgccgttgggccgccaggcattcctcagtttcatggaaaaggtagacggtggtgcgtccagtttctggatgaagcacgaccttggccagggtattgacgattaccaggtaacgttaacatccacgtggaacgagtccacagatgacggaaagaattgggtaatcaccttcacggccaccgctgagaaatcaccattccaggaagccagcaacgcttgtctgaaccagaacctaccagatttgtacgggtgctatggtgattgccttggcgaattccttaaaacctacggagtgtatcagactacattcccgagaatatgggaccccatgcaatgagtcaggaatcagtagaagcggcataccggcgtaagctggcgtccaatccagacggtgaaatggattttattactcttgagatataccacccacttctttcgagacggtggctgctcgtgcgcggggctgacgatttaacagctactcttgagaccggggaggtcgtgacgttcgagggtacgccgatggaggctaagaacgccgccaacaataacgatatggaccagaccgcgtccttttcgcttccggatgtgcttaacatactggatgaggaaatggaccgtataccttacgataacaaggaactgcctaaattcatcttccgtcgctatgtgagtacggacctatcctatccatgcgacggcccggtggtttatgagttacaaacactcacacaagaaaaaggtgtatttacggcggaaactggtgcgccgatgcttaaccaacgggctaccggtattttgatgacaccagaagaaatacctttactccgcgggatactgacatcgtgaatattaatgattacaccggcctgccatatgacttccgtcgtcgtaattgctggcatcacgtccgcaacgtccgcgcggacgctgggttatcaactccaatgtttgacgtcaccagcccaacggcaataggtgcggctttcgacgacggccacgctaatccgaaaggcctaacccgtgcgtttcacccccagaatttcgatgcggtcttgctgggcgtaaaacatagggggcgaatagtgtggcacgctggggtatattacgaaggaatggttagccactgtgagctggcgtccagacaggttagactggatagtctggaagaccttaaagatacttattcggagattgaattttggcgctagtaatccactatacccgtaacgaagacggcacatttgacgttaaacgttatcgcgataatccgatgaacttcgtcgtgaaccacgttcccgatggggtgccggttcgcgttttcatcgacgaaatcggagaagataacgatgtaacagaagacttcgaagcactgaaagaaaacgcgactttccacattgtggaatccgcgggtggtggcgctatcaaaggcgtcatgaagatttttagcgttatccttaaaccgctggcgaaactactatcaccatccgtgaaaggggcgtcctctaacctggcgaactcgcaggcggattccccgaacaacagtctcactgaccgtaacaacaaggcgcgcccgtacgaacgcagctacgacatctgcggaacggtgcaaactatcccaaataaccttatgtctacgtataaggtgtttaacgccgctggtaaaattgtagagtacggctattacgacgccgggcgtggatacctcgacatacacccggaaggtataacggacggggatacccgtgtatcggatataacaggtacgtcggttgccgtgtacgcaccatatacatcgcccaataacacatctacaccacaggtcatggttggggaccccatagagcaaggcctatacattaccgtagaatctaacgaagttgacggcgtggttcttaaagcaccgaacggtctgggcatttctttctcttacatgtccgggtatccgtctttgtctggaaacattggcacgatatatgacccaacaggtggttcggatttttctggggtgttggtgcctaatgatacattttcgctggtgtcagcgtggacaaatacagacgttgacctctccggcggcggatatcaggtagtcagcgtgtccgaagggaccgttacctttatcgtgcctggcggccttatcgggcggtggcaagaaataagacccggttcatttttccgcggtgatggagaggcctcgctgcaaccagacaacacgtatgagaaaacattaaccgattgggtttcaataaaccgtaccgaggttgagcgtatagtcgccaatatcgccgccgcgaacggcatgtataaagacaacggcaaatcgaaaacattggcgtctgtaaccgccgaaatacagtatcaattgctcgatgaaaacagcgctccttacggaccgatatatactgcgcaaggaactgtgtccgggcgtacaccggactacaacggcgtcactatttacgccgacctgccggttgtgtcacgggtgcgggtgcgcgccagaagggtaacagacctggactttaatttcgaggggtctgtagtcgatgagataacgtacgttaacttatacgggcaaacacgtgacaacaccccacactacggcaacagaacaacagtacactcgatgcgcaagcagaccccacgtgctgcggaagtaaagcaaccgcagttgcgtatgattgctactgaaatggtgtacaaatacctcggtaacggtgttttcgaagatacgatgacacctaatacacaggctgtacaatccctcatccgcctggcacgtgacccggacgtgggtggtttaaacctgacggtacgcaacatggataagttacttgccgtgcagaacgaggtcgaagcgtattttggcgacaaacaggccggagaattttgttacacgtttgatgactataaaaccactatgcaggatatagttagtactatagccgacgctatcttctgtactccatataggcgtggggcggatatccttctcgattttgaacgccctcgcatgggccctgagatggtgttcacccaccgaagtaaggccggtacttccgaaaaatggaccagaacctttaacgatgctcaggtgttcgatagtcttaaattctcgtacatagaccctaaaacaaacgttaaagaaaccataacaatacctgaaaccgggggccttaaaacggagacttatgactcaaaaggaatccgcaactataagcaggctttctgggcggcaaaccgtcgccaccagaagaacattttaaagaaaatttcggtgtcgtttaccgccactgaagagggtattttcgcccttccgaatcgtgccgttagtgtggttaagggttcgcgtatgtctacttacgacggctacgtaaccgcggttaacggtcttaccgtagagctatcccagccggttaagttcacacccggagatgaccactatttggttctgaagttacgtgacggtggcgtccaaagtgttcgtgtcgtccctggtgtgcatgaccgacaagtaattatgacgtctgtgccgcaagaagccatttacactggtaatagcgctttgaaaactgaattttcattcggcaacgaagcaaggcataatgctcagatgattcttgtttctacggtagaccctggcgatgacagaacagtcaaaataaccgggtttaactatgacaaggatttctataagtttgacaacgtgcctcctttcggtcgtgcgttctccaacggattcgataacggttttaactaagaggatagccatatgtccagcggttgcggtgatgtattgtcacttaatgatttacaaatagctaaaaaacaccagattttcgaagccgaggtgatcaccggcaaacagggcggtgtagctggcggcgcagatatcgactacgccactaaccaggtaaccgggcagacgcagaagacgcttcccgcagtcttacgtgatgctggtttctccccggcatcttttaattttacgaccggcgggaccctgggggctgacgacgcggataaagcggttctttggccgaaagaagatggtggagacggtaactattacgcatggcgtggctccctgccgaaagttatccctgcggcgtcgacacctcttacgacaggcggcatttcggattccgcttgggtcgcgttcggagatattacctttcgtgcggaagcggataagaaatttaaatactccgttaagctgtccgactttactacgttacaacaattggcggatgccgccgttgatagcgttcttatcgaccgcgattacactttcagtaataacgagaccgttaacttcagcgggaagaccctgaccatcgactgtaaagcgaagtttatcggcgacggaaacctggtatttacgcaattaggtaaaggttccattgtaatagccccctttatggagagtgctacaacgccgtgggtgattaaaccgtggaccgacgataatcagtggataaccgaccccgcggcaatcgtggccacacttaaacagtctaaaacagatggataccagccgacggtaaacgattacgccaagtttcctggtatagaatcccttctccctccggaagctaaagggcaaagcatatcttctaccctggaaattcgggaatgtacaggcgtcgaggttcaccgggcgagtggtcttatggcgtgtttcctgttccgcggatgccatttctgtaagatggtagacgttgacaacccgagcggcggaaaagacggcgtaattacctttgaaaacctgagcggcgattggggcaagggtaactatgttattggcgggcgcacaagttacggttcggtaagtagcgctcaattcttacgaaacaatggcggtttcgcgcgcgacggcggggtcatcgggtttacttcgtatcgtgcgggggaaagtggtgttaagacgtggcaaggtacggtaggttctacgacctcccgtaactacaacctgcaattccgtgattctgcggcactataccctgtatggg

SEQ ID NO: 4 - DNA, 41080 bp, unidentified
  source 1..41080: mol_type = genomic DNA
gggggggggaataagcatggtaactcgtaaaataacagatgaacagttgcagcaagagctaaacgctggtctcgggccgaccgaaatcgcaaagaagtacaacatgtcacgccgtaatgttcagctaaggtcagctcgtctggccaagaaaggtgttggtcacggtcgcgacgtaagccatctggtgccggatgggtataaaataaagggtacgtcgtcactggtggatgagttcggcaatacgaaacttcagtgggttaagactgacgccgacgccgagcgtcaggtcgagttgatgcgagccgtaatagatgggatgaagtccgatattacgccagtttcttcggtccctcggcctaaaaagcggctaaacgaaaagttgctaaatctttacactgtttctgactttcatttaggcatgttggcctgggcggacgagagcggcgatgactgggacatgaagattgcagaagaccttttctctaaatggttcgacgcggcgtttcagaaggcgcctgatgccggtgtgggtgttattaacctattaggggactttgcgcattttgacagccttgatgccgttacgcctgccagtggccatgtactggacgcggacacgcgttaccagaaactggtgcgctacatgattcgtatggttcggcgtgtggttaacatggcgctggttaagcataaatcagtgcgccttcttatcgtgcaaggaaaccacgacgaatccggcatgatttggttggctgagatgttcaacacactgtacgacaacgaaccgcgtgtatttgtggatacgtctccggatgtgtacaagatggtgcagcacggcaaaacaacactgttctttcaccacggccataaagctagattcgacgctatcgaaccggttatgatcgccaagttccgcaaagcgttcggggaaagcgtgtacagttacgcacacgtaggtcacttacaccaccagaagattgtagaaagccgtaacatggttgttgagcaacaccgcaccctggcggctaaagacgcctatgcctcccgtggcggatggatgtcaggccgtagtgcaaatgtaattacttatagcgccgaacatggcgaggttgcccgcctaactatttctccggagatgctgggatgacaaataaatacaaccgtacaatgacaaacactgaaggtgacagcattacctgtgatgtgtacgacgttctgagagcctttgatatccgcgaccctgcactacagcacgcattgaagaaactgttgtgtactggtttacgggggcataaagatgctgatactgacctacgcgaggcgatggagagtctggataaataccgactatatctgagtaatcttgaggaatgaaactaaggccccgaaaggggcctttttattaagcatacttaatgcgctggataacaattgaagactggaaccggttaccggttgcctggccctcaagagaaagggtaatagactccgggccgccgatttctggggttgcggacgtaagtgaagcacgtttgagtgtaaagctcatagcgccatccggaccatcaagaaccgaagatacctccatttctgtttcatccaggaatttatttaacagcgtcaggtcgtacagtttacctgccagcgagaaggtgttggccgcacgactgcgctcaacaaatgccacgctgttatttcccaactcgaactgtgcggatgccccgttgtcgttagtaatagtgaacgtgtcaataagtttaagcggtgcagcaccgtcgtaaacagatacatcaaccgaggcaaacgggcgggcgctaaaattggtcgtgaagtctgaaccactcggcggtgcttgtaagatttcctgattcaagccgatgaacgggaaagaaccggtgaccattgcgttaacagcctgttcgatagtgaacccggtaaactcaacgcctctggttacgatatatgaatccgggtttccgcacttacctttcaaccacgtaagaatcgaataggtcttacacaagttaccggtttccagtttatctgcgatacgcaaatcggcttgggcgtctgcttgggcggttagagtgtgctggatgccagcaccggttacaaccgtagcggttaccgcggtaacgaggaaagctttatcgttattaccggacagaccatcgaattgcaccaggtcacctacttcaacaccatcggttacaaagctaccggtagcccgtgtaaaagttttcgctaccgggtctactgtgacgccgattgccgatgcggtggaacccgctacccatgaactagccattgcgcctgctaacagctcgtcttggctttccgcacttagctcaatagcgtactcacctgttacctgacggttgccagtgcggatagctgatgtttcgttgctgccgtctagctcattagagatgagggcgtctcgggttaccgcggggatgccgcctgtgttgcggagtggctgccataccgggttagctggcgtcacccccggggtttcctcacgaacgtaaaattgcgcggtcatcgcgcctttatatggttgtaacgccattatctaatcctcgctgtaaatgctatgaaattaattgagagaggcctcttcgcccatccattctcaacaataagaggaccgaggcttactgattgtacctcagcacagatttcgttacgactaaaacagttaccggccgcgaaggtagcgtttagcttatcagctaatcggttgataggggcactacctagagctgacccaacgttaatgtccacctgataaacacccgaccgttgttcagtccagaacaagtcggcttgctccgtatcggacaacagcatataacttgcaagatacggcatatcagtagacgtcggcgcatcgatgttctctaacgcaaccttgatgtcgttgtccatgccgaacgtcaccagcgccgtgtcgaatgctttcgttaagtcctcaaaatatgttgccattatttcaccttagaggcttcttcatttagtagttgctggaacctgctgacgttagtcctgaccataccttgcggggcctgttgcgaccaaccgtactccagacgttgtgcatacggtaaattgttagtgagggtgaaggtatgccagtccgcagcgtttagcacgaaactcgtaaccttaccggttgctgcgttgcccgatttatcggtagcgtccgtagtcccatcggcgggggtgcttcctgaagccatccagttcatacgaaatcgcccggtatctacgggactcgctttgataatcgcagaaaacaactttatagaaacctgacgcattaccttctcaggattcttcttagccttttccacgaacttggacacatcaagcgcaaaactcattttctcacctgaataaagtatgccacaacatcatcgttaaccatcttcttctcaatagcaactacagaccattgttcaccgctaaactgcaccttgtcatccatcttcggaactacgctgtaatccgctttaaccatcacatcgcctgcctgtatagtagttccatttaccagtccagcgttaacgggtacaggaaccgctttaaggggaagtacaacgtcgggcccccactcgtactcccctaaaacagggtcccatgttttctggcctttgcgcaccaaagttaccgtgctgccgtattttgatagtaggcgagtacctacgccttgcatccgtttactaaaagcggtgctcatcacacaccctccagtcttgaaacgaccagtagtgcggatggggacacaccccaggacgtaaccgttgccacttgcgggtataacccgccgaagttggaaccggaggcgtcgcgcataatctctaccgtaaaagtctgccctgcggtagcattcacaactacacgagattcaatggggattgtggaatccgcgctcgccagtttagtcgcggcgggtgatccgaattgcacaccacctagcaaaacccgcgacagaaggatagacgttccgctcgccccagtacgtccggcttgtaacttaacacggatggcgtagttgcccgcggcgttgaacgtaactacacccgacgcgttaatcattaccgggtcggcgcttgttttctgcgcccctccaaaggctacggttaatgctgtacccgtggtggtcggtgcctgaatatctgtagacgacgcgcgaaggacttctacttccttagcaccgtacagcatagagtccgccatctgagtagtaacttcgcgcaacttttcgggtgtaattagcccggactggttatcagggaagtttgccccaatcagggcgaatatttctgatttagtcttagccatgtttaacccctgaagacattaaaggagtaggcattattgctcccgcataaaagaggacgtagcgcgtcatcggctgccgttatgcttactgtaccgcctgagtagccattcttaaagtaggacacggtcaccgcgccttctacccgctccgtctgcacctcgcgcccgtctgtagaccctcgaacgtccgtacccgcgccgtactcaaccgcggccatcacctgagcttgaataactaacgatggtattacgttggatggctgcgggaacccgtgcaggtcgatacccgttctggggaaagccagtgcctggtttgctgaaacgcgacgcccgcacatctgagactcaaaaagacccacgtataccgcgccattacggagagaggcctccgctgcgatgtcatcttctggtagttcgagaccgtacttagcggccagcgcccgcccgtcttccaggctaacataagagtcggcatttggcaggccttgaccggtttccacgataagcggcataaattattcctctacacttttacggcgacgacgttgcttaggctccccgccaccgttgtaatgctgttcttctgctgtcggctgagtctcaattaatgcgcccgggttcgcttcttcgcgcatcggggttaactgcccatcaacctcaaccacacctttgtattgctcccttactacataattatctgccatgtctaatccttaaagcggcccggtggccgcatgtatttaacttactgtcactacagtactatctgaaatgatattaccgtagccgtcgtgcgctacgactttgtaagtaccggaatccgccaccgcggagttggccttagtgtaagtcaaagcggttgcacccgcgatagcgttattatccttataccattgaaccgttttagggtccattccgtcggcgagttccaccgtaagggtgatagcctgcccagcggttaccgcggtagtatcattgaggccggtggagaaacgcaacggcatcacgttctccatatcaatttcaacctgcccgtcctgcggactatcatcagatacaccaacaatacgacgtttaattacatcaaccatttttatcctcctcaggatacagtaccagcgtttttaagggcggttaacagattagtaacggcggtgcgcaatgaagtcacatccgtacgcaacttattgtagttggtgaccagtgcgtcgaactcttctttcgtaggtgcagcggcggcggctgcaccgctacccgcgctaatcgccggtggtgccgctaccgtagcggactttttaacccctccgattgctgttgtggtcgccgcgggaatatcgaattccgcgttaacaaggctgcgtggtagccctttacctgtctttgacatcatgtagtctccatataaatgaaaagagggaccgaagtccctcaagagtataccctacgattaagcaccgacgccagttacaaggaacgcaatcggcacatgcttacggtcaactacacgattccagttggtagcgttagccaggtcctgccagctagcggaacgtgcgatagtctcggtaccgttgccggtgattacagcgctggtgaagctgtaaccgaacggatgcagcaaccacgttttacgcgtccacagggtttcaacaccaccaccgttaccgcgcgactcttcgcgctcatatgccagcggggtttccggggaaccttcaccgtaaccgatagcaccacgtccaaagatgatggagatgaacttgcgctgtgcaccctggcctactacagtcatgctgtcatcaacgatgacgcgatagccctggtaggtagcgaacatggtattgttctcagcatcacgaatgaaatcgataagctgagctttacgggcctgtgcatatacgaagctgtgcatagcgatagcaccgagaacctcgccaccgttacccatcaacgcatcgcccatagtctgagtagcgtcgatgaatgcgcctgcgtcgaagccaagggtggcggagacgtctaccaccatgtcgttctgctcgtggtattcgtcggtagcggctacgttgtcgttgtacaagccgagggcggtagcaatcagacggcgctgtgcttgacgttgccagaagttatccagacgggacgctaccgactgcaacgggttctggctggtcaattcgacggtcaggtcagcctgaccgaagccttcgttcaggtaggcgacgcgggccatcatctcaccggtctgaatagcgcgcggagtagcgatatcctggtacacatcgttcgaatagttaggttcgatagaggtgtcgatagctctccagaacggcaagttagcgatgttggatggaccattagcaatctcggccgcgtacggggttggagtaaggataccggagtcgaaaaacgcggttttctctaccgggtcttcggtcatgtaagacgccagcacagggatattgccagttacgatgttgccaatagtggtaattgccattatttatttcctcagggctttaagttgccgttcaaattcggcagggttagatttatagagcgctaaacgctcactttcactcatgtctttaaacgctggtgcggccccgccgcctttattcccggaagccccgccaccggaagctgcatttgctttaatcaaatgtgaaaaagctttatgttcgcgcaggtatttgcggaattgctcagggtcggtagtaacaacattgccatccgcacccatgaatttagtcactacgtcttcgccttcaaactcggtcttaacgaacggcgccaggatttctaccgcttctggggtgataaaatcaccagcgaaagaacctaaaacagccttacgttcactaccaagaattcgcgcggctaaagaagcatagcgttcttctttctctttcagtacgggttcatactgactacgaatcgtcttttcaaattcgtccattttaccggcagccttcagtgcttcctggtgggcgcgttgccgttcttcttcggcttctttagcccggcgcgcagcttcctttttctcgctaagtaacgcttcctggttagccttaagcccggccacttctttctcaatcatcgcctgcacttcttcggccgtgtacatttttggtgcgtcaccgccgcccgcgggtttaaggtcatctggtgcttcgtcttgaagcgggtaacgtaaaaaacgattcatagtcaatatgtcccctggactttaggacaccgggcctcccggtgtttctgtaacaagaataaattattccacttactaaggcaactattccagaatattccttatgtactcctgcaaccggaatactttcaggcgcaattgccgtgtgcactcggcgttctgtacatcgatggccaggtcttcgtcggcatcactacttggcaaggccattttgcacggcggctgcattagtgtcatatccggggatggaattagcattctcggcggcgcgccgtttgattgacacccgctcaggagggaacacgcaaacaacacgaccaggacgggtaacgtatttgataacttcacgggtgatagcctccgcgttttctttaccttcggattcggcggcggcggccttggcctcattctgctgctgccgttgtgttttcttcgctagctcggcctgtgctttctgctgttgctgtgaaacaagattggcgcggccttctacccaaccgcttctgtagttgtactggccgtataaccacaaggagaggataccggctgccgtgactaccgcggttactttccaattcatagtatcccctttctttatacgctaagcccggcttacgccgggcgttaaactcgtatatctgtaaaccttatacggcgaatcttagctaaatactgaaagcagtatagctcggcccttccgaatgcgttcgcggttacgctagggcgttgtgacgcccgcttcacgcgacccgcaccgcggtacgctgaaagcgtagttagcgtagcatgtgaaataatataagtctactttttgaataaagtattccttaatttacattattcatgaataactggaataatcgcggataaaattaaggccccttcaaggggcctttcgttacttaggttccgagaccttagccgctttgctaaccaccgacacaccgtcggcatctactgccttgacgaaatattctccggggtctttaacggttagttccccttcaacgtaagggacgttgattacctctttaccgtctttaaaccattgcaaatcgtaaggcgctttgccgcctttcacaactacggttagcttagcggaaccgtctttcaattctacatctgctggctgtgtatcgaagtacacatcacccgctccgtcccgatacggcacttcgtacaggataccgtcggtgattgctaccagtccggtcttatcagcaaacggcatttcatctaccgattcaccgagaaccgattcatctttaatgtacacgacaccttcgcccacaccggacacacgggagtactgaaccacacgtcgcgaaggcacatctttaactttgaaaaagcccatctctattcctctttcagataatcggcaacacgcttatcgagttccgccatctctttaagagttaacggacgcccgaatccatctaccgatattacacgaaattcttccggggttatcccactattacgaaaaatcttacccctgacagggccgagggcttcgtcctggaaccaggccggctgttgtttaagaaactcgtaataggtggtgtccgcacttacctgagtgccgccgtccgcacccctggccgcccgttttgcgcctttatcaaggaagtcgaattcggaactgattacaggtgccgtggtacttcggcagttaggatgcgccgggggcgtcggccccttaccgatttcccacgtcattccgtccctggccctgcaaatcgtactggtacggctgtccagcgttgacacccattcgtacttctcgatgatgtcaccgttctgccggtacgtctcgttgcgggcttcgttggatacgtgggacagtgcggtgcggattactgtagctgcgttacgttctgaaatgtccgccagcccaccagcaccaacaacattcttaacaatctgccgtgtggtttgaccttgtacgaaacccatcttaacgccagttaccaggcgcgcgacttctgttcccccccagcccgacatcagtttagtgaaatcgacaggtttgtcgcttagtgacagcggctgaaactcggcagcggcccacacctgttcggcgccaggcgtaacaaattccgcgtttacgttggcgaccagtgcctttacattccactcggcctcatacgccgccagttccttaaggtcttctgtcagctttgtttgccagtcatcagtaagcccggttagcgctttttcaaggtcgcgtaacagtttattaagccgcgcggtgcttcgcctgtcgtcaccgaatagcagcacctgccgtttgatttcgccccgcatctcttgaatgaacggcgcgaggtctttcacttcgccggatgcggtacgttgtaaccatatctgatggctgataatcgattctaaaagactcatgtacccctcctgtctctacgcaacaatcatatcagccagtagggtatagacataatcatgcgcatttaaggttaaaaagagcattgacaaatggtatatctatatataccatttaatcatacgcatttaaggttaaaaagagctcggttaaattctacatttgaatgtagaatttaaacgtacacttaaacaggagtgatatacatgaaattgcaaacattagagttcccgtatggagaaatcactttcgaccgtgacaactttattaacgtaaacgagttggtcgaccagatgaaccgttggcgcttagacaatggtatgggctttaagcacctacggcagctaatgacgacagatgggtttaaagatttcaaggctgagtgtgaaaagcagcttggcattaaggatgccgtcaggattacacgagggcgaaacggctccacttttgtgtgccttcaccttgcggtatatattgcggagcaatacagcacttatttccatttcctcgttatcgaccgttttattacccaacggcaggtcgaactacgaaacattggcgcggttagctttgtcgagcttaacgccgccgttagtcgaatggttgagcgcacggagggaagaatcggccacagtggacacttcatacaggtggctaaagcggtaaaggaatccgtagatatccgcgaagtaacgggtttcgatacctgggactctcaggatgcgaagaccaatcagttgcgaagtgacatccagaaagccctggtgacgcttcttgacatggaggccattaatacatggggcggactgaaagagacgatacctcgggttgtgcgtaaatgtgcggctaatatacgttaaaactaaggccccataacggggcctttctgtttactgctcctgttgttgcaccgattgagggatttccccctgaacttcagtagcgaccggtaatggctggtcggctaccgcatccttaatatcagcgtctgtccagtcggtgacaccggccctacgcaacgcggcatagtaggctgtaacaggcagaaatcccgcattaatatccgccatccatgcagccctgtcttgagctgtcataggttccaggaagaagtccatattaaggtggaattcaacctcggtatcttcgggtttacccaacattaccgccacccaacgtaaagcatctgtatatgcctggcttacgttacgcgcaattgtcgccatgacggatgtatcggcaccgcgctggattcgggcagattgtgcggtgatttgctgagtcggggtgataagctgcgcaccaatctggatagcctgctgctctttgtccagcatattctggcgggccaggctattttcgccggcctggataagctgtgcgctacctccgtaccccaggttatgcccacgccgactgccaaatttgataccgttggggttcgcttctttgaacgactgcggtgttaagttctctcctgggtagataaacagcgtgggttgccctacaacgaagctggactcctcattgtccgcgctgttgcggaaatgcccgatattaagctccgccagcggcaacaaaggcgcgtcgtcgatagtggcgtcgttattggtcgccccaataaaggtaaacggaattaccccccgtaatgattcccctaagtccgggtaaatctccacgacgtcttcctgcgcaccaccttcggcatcgaaacgaaaaagacgttgacggtagttgccgtcgctatcgatgtccaggacccggtactgttccccatattttgtttcgaactcgtttcccggttcgtggtactcccatgtctcgcgcagcacgaccatagttacccggtttacggaaccgacgcgcgtgagtcgccagttaacgatgttctcagtcgtgtaaaaagcgattgtaggattaagcagacctgcgttctgttcagccgccgtagcagcgccggtttccggagcgtcgacgagaagaccaccacgacctaccgagtcaatctccatgagtgtatcttgtgcgtgctgtatcaggcctacaccagacccatccgcatttttaagcaggtattccagctccttcgggatattgatttccggctctttgcgcatgacgctaccgaccatccccgacagcgtgcgccgggtgaagttgtatacgatgccgccagcctcatattctgcctggcgcgcttccccgtacgctttatctggttcgttcagtcctacgttgcgtaagtagcccaccagttcacctgcaagcgcgtgacgcaccttctgccacttaggggagtaatgcaaccattcgcggtgcttggtttttacgccagaaccctggccgtttgctgttaacatttaataatcctcttaaagtgcgaaagtaaccgggatatgggatacaggtttaaccaacggcatttcgtaggcgatagggtatcctgtcgcgtcgttctggtggtcgttgccgcttgtcttatccggtataccgtttttatcgtaggcctgttgctccaggcaacgtgctgtaaccgggcaagcctgttcgttgaccattaatctaccagactccagcgctttattcaccgatgctacacggtctttaacggcagggttaaccgattttgcacgtatctcgaaaccagcgttatgcagttgtgcaatatccgaggtacttgcatcagtcgatttacggttcttaccactggcgtccggatacataacaatgtgatgcccgttacgcttccatcgttcggtaatctccctgaccacatccggggtgtcgaacatatccaccagctcggctaccgcgtgccagacatactcacgctgaacgtatacggtgctagccatatgcccgacgttgaagtcctgaccgatatacagcgtttccccaggttgtatagtctctcgactgctgttcttgcgtcggtcgtaagcataatacacgctgccggatgtcaggttgacaaattcaccatcgatgtaggcatcaattaactggcctggatacgtatcccgcaacgactgcacataatcttccggtagaaaggggttggatgttgtcgaggcctgaatcatttcatagcctggtttcttttttacagcccatctgtcgtgcacaaaacggaagccttctggcgtcgtaaataccgaaactgtattagcgggcttcggggtaatcggacgatatgtacgcggcaactgacggttacgggcgataactttgttccaggcgtgctcggcgtggtctttatttaacgtatccaattcgtctatttttgcgcgaaacgattcgtagccaacaattcgcgccgggttatccaatgtacgcaggacaaaatccccgaattgcccggatgatgtatagattatgttgtctgatttattgtacttgtaacgaataccccaatcagacaacttctcttccatacgcggagcgaggataaggcgcaccaggtcgtatgtcggttcatacatggcgataagtgaatcactaccaccttccatgctgtcgagtagggcggagttgcacatgacctccgatttacctgtgccgaatccagcgacgaaggccgggaatttacaatgcagattaaggaacgcgccttgcggttccgtagccgttatatcaacgttcaccggacaccaccttaatagttacttcgctaattggttcgtcgcgcgcttcttccacaacagttttattaagcccaagttttgctgcggcgaacgtagcagatatcccggcagcaccggtctcagtgaaatacgcctcttccaaggcctgcgccgtttcgtatgcttcagcgaaggcaggaacctctcgcagccacagcttgataatcggaacggttacgcctatgtgtagtgcgaaacgggccagcgacggcggtttgtcctggataagcgggcgttcgtcgcctttagacgtgggtacgagttcccatgaggtacggtcgaagaaccggattaactcgtcgcaatagttcgggtcccatagttcggcggaatgccgggatgactggtacagactttgctttacgcgcgggcgtttacggcgacggtttgcactaaccgcttcttcgtgcgcggcggccactacttcggcgtccggctgttttaatttgagtttcatgcaatcctctcttaaatgcgtatgaccatcatagcgcatcagacgccccctgagcgtctattaaggattgtacaggggaatgaaggcctgtgtacagaaaagcccgcagaagcgggcttggtggtaggctattttacagttctacagatgataaatggttggccgcggacttaaccgcgtctttaaggctatcgtacgcggaagtaaaagacgtatgtactcctttcgagacaactatagcgccgccgttaaagtacgcttttgctctccagccaccatttaccttatcaataaggatgtagtgactaccttcttccggtaccgtaaccgtgttttcgtctattacttcaaatttagtgcccatgttcgccctctcctctttagcaatgctcgtctttataaacccaccatttacctacggcttcccatcgccacccgcgactatggatggcatagaatctacggccaaatactgtttccccgtaccacatgacaaatagcttcaccgcggtagccgcaccaattttatgagcattcaaacggcgatacgctagtgacatcctcgtaagacttaccgcctcaacaaatggccaaagaaacagaatccacatcaggtatataacaacggcaagcagtaatgttgtgaatcctatacccatgaggatgagaaaactatccatcacccctccagcttcatacggtcgttgcgcgatagtccacgtttcgtgtagatgaccggtgacgttttcgtatgggtcatcagtcggttgttatagataacatgttgctcgcattttgtgtcatggcggtacttggataatgtcatctcattaagtcccgtctcccggcacgtatctgctaatgtgccgtgtgtctcgattagcttggggatactggtaatcattctttatcgcaatccttcggtttattcaggtcgtaacggttaataagctcccggcgatgctcgttcaggtagtctaatgcggcctggtgcgccttaatttgcgcgtctaccgcttcaatcttaaacctgccgttggcggcttctaccatcatcttttcatattcacggtcggtcataaaactcatcctcggcaatggtttacgtctcattctttacgcgcccccacatacggttgagatatttgttctggtcaggccctgggaaactattgcgttttaacagttcctcgcgtgtcgggaatggcgtatttcctaccttaccgcccaccttaaacgttactgctacttgcccgttgtcgctcattcatgcacctcccatccgcatacgatatatactttcatttttgccccctgcataagctgctttcagtgtctgcatcgcggcgaaccagaacgcgtcagcaggaattcgtgtgctcagattattaacggcaatgcgtgccatcaattgcgcgtctttgaatgcttcggtgtcaagcaccggtttgcaatggtaaaaaactttatcgctttgtaagttcatcttctctactccgttctcgctgtcgatgaactaaatataatagggttaatgctaccagtcaactattatttcgggatacagataaaaaaaaatcccggtaagggtcagttaccgggataaaggagctagagggatgaataagcaggagcgacaccagtatcttaggaatgctcatatgtgtcaacctactttgcttcgcggccgatggcttccgcctgttgccacagcataccgtcgaatagcgcaacacgaccggcggcgcgacgacgtaatccgaggagtgatttgccgttctgataatggaactgagttaacttattccgcagtggggcaacgtcgcccttacgcagggcctgccctgttccggtagaagccgcaatcacaccggcgccagcgttatacaccaggtcgcacatcgcatcgaactgtgactgattgagcgtcggatgcgcaacggcgtccaccgcagctacggccttagccatatccttatgcagcaacagaagaccctggccctcggtaatcttctggccttctttcacatcagcaccgtagtgcccataaccaatagtaaggtacttctcgttcttcgttgctttgtacgcggttccacggaaaccctcgaacgcggcggtgaagtgtagcccgttatcactgatgtttcggtttgccatacatcctccggtatttgtagtggcgagcgcctttcacgcaaatgtggcgtaccgatttaagcgcgaatatggcaacaagagtcgtaattatgaatggcgggatgtcgccggtaaagagatgagcaagaccaccagcggcggtcactacggacgccatatacaacagacgaccgaaaagaccgtcttcaatccagtgagcgtagatattaattaacgaggtgatgatgattgcgaccagacaaatgacaccaattggtgagtcgagcatgttatttcccccacggtaacttaaagttgctgagggacgagtcggcggcctcaaataacttaagccagaacatacctacgccaaaaggaaccaggtactgaccctcagattgcgataactcatagtaacttataattatcggggatgcgtacacagcgcaacaggccgaagcggtaagatggcacaatcgcaaccatggcgaaatagcctcacgcttcttaatctgagacacggcacccccggcgaggcccgccacactaagccaaaggtatttatcgtccacaataaaaaatctccgaaagttggtttaccttcggagattaacatttatacgacaattttagtaacagataattcggatttatcctagtcgatgtgttatcactcccccatcagtattagggtcgaagcgcttcgctatctcgacagcagcagtagcggcggcacccataaacatcgccgtaagtgcatacggagcaccggagccaacggccgctgcgttacccgcaattggtataactgagcttaacggcgaatcccatgacttgaagaattctaccctgaaaatacggttatcctctttaacaaacaccagcgctgaaaagtcgaagtcgtagtacttaggcgtcaatatctcggcatcgacaaccaacagcctacccaccccggcgtccccggatacgcccataacgaagtggtcgttctcgtaaatcttggtatccgtatagtatttaaaattccccgtgacacatgtatcgcaggccatcgtttcaccgtcaaaagctatagtagtcatataagttacctgaacgtataaagtcgaatagatggcttgtgcgggatatcggaatacccacgtttacaccacaacttatacagtttaacagccatatgcgcccgggcctggctactgaactcaccgaggtttaccactttcttgccgtcgtaagagtgcgcacgccacagacctttgcctacgcaatacttcggctgatgcacacctggtatacgcggagacacatccggcatctcattagctggctttactcgcggcttaggtttacgtttacgcttgtcttcttccttaggacgagtaaccttctttgttaccgccgatgaaggcggtgcaaggttaatgccggcgtgtttaaggatatcggtcgcgagactcatttctccccctgcttagataaccgttttagtgtacagataccgcgtacctgagcgtcaaccggacgctcgatgaaggttaacggcgcgttctccgcggcgcgtgcggctacctggcacgcctctaacgtgtaaaacgtttctgtcccggccagttgtagctgaccggcggacacgacccatatgaaaagtatgcttgtcatttgattaccctgtacttcacaatatcccagttgtcgttatcgattgaccacaggtaatcctgggcctcacctacggatgcaatgccgttgctgaattcgacctccaccttagaatcgcccggcaccgggcactccttacgtcctggattttctttccaaccttcggaagattcgtgtttatcgcgtgaagtcgcccggttgaagcccgcatccatatgctcaatagtttctcgtaactccttaaccttatctttcgcccggcataagcgggctttcagcacatgtatttcatcgtccttacgggcgaggtgctgtttgaggttatggatgatttgttcgctgtcagaatcccggcctatgtaggcgtcgataatcgcgttctccttgtctgagtaccctgagcggcctgttatcgaccggtctatgctcatcttgtgtatttcttctacaacatcgaacttacttacacgatgctgtagctgctcaatcaggtactcaagtacttcgtctttgttattcatttctcttagctcctctcgtttcgataaagtaatagtaccctattatgtttggttacgtcaatactattttacgattagtttatcctgacgcatcgtaaagtaacggggcttcttcgcatgagcgcgccaccactcaacgcgggcgtagatgccctgaagcgatgttactttgccgtgtctgatttctccgttccgcccggtccaggttacaaggtaatttggtttcattcgttagtctccgttatcgatgcgatgaaggtcatgagttccttgtaacaagcatcccacctcatagccgctgcttcatactcctcgtcgtcgaccctgattgtataggccatatccgccgatgcatcgtcgatatcatggacaagtttcatcagatatgttgcctgctctttagtaatcattgttgcgtctcccgcaatagttacgtttattgttgcatttcgtgaaatagaaaaatattaataaaaatggtacgctgctactcagcagcgtaagtcactcagtcaattcctaaatagttgttacaatataacatcacacttccggtggttccggtagcggcatccgctcactacatttaatccacttactcataactccacctttatcgcaaaaacttccacgggttcactgccgaaatgcgggtgtgtgatttcttttatctcgtacccacggtacttacactctattctcctgctcgcgtcatcccgtttagggtaacccttcgttataatcaaacggtcgaagtcacggccaaccaggcgcgtccaccaataatcatttaccagacgatactcctcggtcttctccccgctcttaatagcgtcaaaatattcacttttcacggccaactgtaagtttttcacaaacggcaatctcctttaagattcacgacccgaagacacagactcttcagaatcttttgccgtgtagttattccctattagtctgacgaagcgctggctcatccacacgatatgcccgttacttaaattccctacatagagcaacgacccgtgctggtgcagcactacgtaatactccatagagatggtacaaattaccaactgacccttcttaaacatgattcatcctcttgtcccgttaaccttgcaataatagtactctattatactgggttacacatgtgcctttttattcttctctctcgtccctcgtcaggtaagcgcttgccgtaaggcaagtgcgttaatccctgagtagtagaccaagaatctttcggcatttcagtaacttaaaggaatagctgccgatgagaatatagctcgcccaaggcagttcagatactaaggaaggaagttacttaattttaagtttgcgggtttcagtaagccttaacgcgtttccgcttggggcggaaccgctaagctctcacaaggtgaaacagaaacttaagtaacttgaagttacaagagacaacttacacgaaaaaaatgaaaatgtcaataggtgtgtttatagttgatttcccattattccgtgtgtatacttgctttactaactgagatggaggatttgagaatgacacagaatgaagtagctgagcttattggggttacccgccgcacgctgaataactggttaagagacggcaagttcccggattgttgtgtccggattatgggtcgccggatgccaggcacatttgaccgggagaaagtggaagcgtggattagggagaatgtaaagtgaccgatttccagaaaagggtattcagaactgtagtagcgcttacatgtaaaaaagggtcgtgtagtgtccttgacttgcgccggacttattttcagtactatagttcctctctcattgagaacacgttgaaggttctggttagagacggagcagttaaaagtaaggccggaaagtattcggcagtagctgaagttaatggctcccaggcgaccccagaagaccttgaataaaagaaaagccccaacgcgcgaacgtcagggccttaaacactacaaaggaaaagcacatgtcaacaggcaatttagcatactcatggtcgcgtcgcaatcgccgtgaggaacgcgctgacattaacgtctacaccacgacttccggcacctgggatgagttcgttgaacttatgcagccgcttaaacgttcgcgccggaaccccaagacagaccccggatatatcaccgccgcgtgtaccgccacggtaagctctactggtaaagaagccgccgaaggtatgttctatcgctgcaatgcgtctgttacgtcttcatcactagcctatgccgacgtggacagcgcgacgccggaagagttcgccaccgactgtgagatggtgcgcgaatcacgtttcgcaatgatgctctacaccacggcatcccacaccgaagaagcgccgcgctatcgcgtcgttatgccggtgcgcactccggtaaccggcggcgacatcattcgcatccggtacggcctgttggcacacttccttaaagggcgtgatgtggatagcgccgggttcaccctgtcgcagcctatgtaccgcccgccggtaggaagccaggtcatcgtgtctgaaagcggtcggatgattacggcgagcaagcttatggagggagttcctgaaattaacattacgggcgcttccgattataaagtgccggatggtgagcaatccgaattaaccgacctgttcgaagaattcgcctttgagttcggcggccgtattaccgaccgcggcctgcaaatgcccgccacgccggagcacgccgcccaatacactaccggcgaacctaagcaggatgacttcctgttctgttggccgcgcgacggcttcgaacgccctaatgtcaccctgtaccatgacaccgacctggtagctacgggcgggatgaagcctggtggacgggatatgtgggcttacgcttgcgccgctaccggcctgccgttcgaccgcgtagaggtggcgcttgggtgggcggagggggtcacttgcgacgaagaggacctggccgacgaagaaccaccagcaccacaagcggacttcatcgtcgaagggtacatgccgtctaattgcatatgggatatcgtcggtgaatcagggacgtataaatccttctacacactcggcatgatgtatatcagtgccgcaggataccggttcgctggggcggatacccgtaaggcacaccacttctacatcgatggtgagggtggggagttcacgcacacccgtatcgctgcgttggccgctaaatacggcgatgaaggtatgcggtatatgcatgtgcttgacgctggtgaattcgcggatacgaagaaactggtgcgtaaaatgcgccagatagcgggcggtgaacctgtcgggatggtcgctttcgacactcttaaccagaccttcgggaactggatagacaagttcaacgagaactccgccggtcaggatggtatgggccgtgtcgtggctatgcttaaagaagtacgcgacggtactaaaggggcggtaggtgttgtccaccacaccccgaaaggagggagtaaggcgcgtggaagtggtgcgttgtatgccggtgttgacgtggaactgacacttgttcgcgccaccgagaaacaaataaacgtcgcgcacaccaagaacaaaaacggtatgcagcagaaaacaataggtatggtgcttgaaccggtacagtttcgcgaagctccgccgccgaaagagttccaggctgtagaattcgttgggggtgagggatacggggaaatcgttaacctcgacctgccggaaccacataaagctctcgtacttatgccgtggggcttccagccgttcgaaacggacgaggagaaagagcgcaacgaaggactcgatggtaaaggtaaagattccgtaaaggatactgttaaacggtctaaagatgcttccgcccgcgaatcagttatgtctgctctcgaagatttacagcaagccgatgatacaggacgtggcttcacacaacggcaaatagtcgcaagagcgggggaccacagcatcacaaacctggtacttgagaagatgctacgtgagggtgaattgatgcttggttgcgatgaaaacggtgaagttgtaacgaatacttacaggttgccaaccggtatagacgaccgaaagcggccaaagaaccgatatgaacctaacgacaacataaagacaacagaaggtgatttagagtaaaaagaaaggggccatcaggcccctgtatcaccaaaacttaaccatctagggtcgacaccgagggcatcggcaatatgaaagattttaaaacagtctaccttagatacccgaccggttgccaggtgggtaatcaggctttgggatacaccagctttgaaggcaagctcggtctggcccatgccgatttctttacggcggcgctctacacgagcgcctaattcggattgctgcatatttttactccttagttagatatgtagtaatagtacagtaataaaatacacgacgcaatttacgaaaaagtgcttgcataaataatagagcacgattatagttcttatcaccaactaaacaggagaacaaataatgttagagaaattcttggtattacttgaacgtttcgtagttgcccatgaactgattgctgcgaatagcgcaaaacagatacctaacaaatccgtaagcgaaatgactgttgacgtctccgttgcaggtgtcgagacagttaaaaaagaactggctaaagcactggaagaagtcgagaaagcaaaagaacttccggtagaaggtgaagacgtcgttgacactaaaccggcggaagaggagaagccaaaacgtaagccacgtaaggctaaagcagaggaacctgcgccggaaccagaagaagagaaggaagaaatcgattaccagtctcttcgcgaccagattcagggtatcgatgacgcaattggtgagggcccgagcgatgccgcgtgtcatgattccgatgacctgctggaagagttcaccggtaagaagatgaagattgccgcgattaaagacgaagacctggctgaatacctggaacgcatcacggcaatcaagaacaagtatttcgaagaagaataattacccggcggcccttcgggccactttaactgagggttaacggatgaagagtaaagtacttatagtgactatagaaacagcttgggtcggagcttcagaagttgtagagatagaattagatgggtctgagactgaagaagaattagaagatatggctaaagaagagttcggtaactattgcaactacggttggagggtagaagaatgattcttagagaacgcgccggcaataacgatgtacacgccttattgtcaccatccggtgctaagaaatggttaacatgccctgcgtcgctgatatgcgaaaaagatatccctaacacttccggcaaggctgcagtgttaggtacggcaatgcatagcctttctgaatatcatttaaacgcgtatatacgcggcactgagctgccgttagagcgcgacatcggtgcgtatgtactggaagaaggtaaaggcgcagttaaagcactaattaagccgatgaaaggtgcggttcttattacggatgatatggtcgaacaggtgcgaaagtacactgactactgtaaagcggttatcgatgtagcgacctacgccaagcttgaaatgcgcgttaatcttagtgaggtgttgcaccccggatacgaaggcgttgagacgttcgggaccgctgaccttgttgccgtacaggaattggctaacactgacgatcacatgctcgttattggcgacctgaaaacagggcgccatagagtagaggcgaaagaaaacaagcaacttatgctctatgctctcggtgtttaccgccgcctaaagagacgctataacataacagctgttcgtctggttatctttcaaccgtacaccggtggcgcgtcggagtgggatatctcagttgaaggtcttgaactattcgctaagttcgcgcagaaacgtgcggtagccgcacttgacgcgtatcgacgtggtaagaaaaaccttaaaccatcggacttcaagccgtcggttgatggttgccaatggtgccgtttctcagaacaatgtgccgcgcgtacaaaaaccgttaattctgtactggcggaagaactggaagacgatacggatgtatatcgtacagccgtagaacgtgtagctaagttctgcaacgacgcggactatatgagcgtttgggaggatgttcagccgagcgatattcttaagttgctgcctgatggcccagatttacctaagaagccagtgagggaggacgactttgcactggagcttacgccggagcaactcgtcgctgagtatgagaagttgccgttgttgcgccagcacatcgacaaagtagaaaaagcgatggcggccgcgcttcattccggtaaaaaagtgccggggtacaaactggtcgagagccgtccgggtaatcgcacgtggaaagatacagatgctgtaaataaatttagggaaaaccatgtaaacggctatctgctggataagactgttccggtatcaccgaccgaagcagaaaaggttattggcaaggaagacccagaactttgggccgagctagctaaactcataacccgtaagccgggggcgccatgtgtcgcaacggatgacgacaaacgaccggaatggcaaaatgttacggaggaggatttgtaatgaagaaatttacatggtggttgctagcagtcccgctaataccgatggcttttatatactatttaatgagagcgttagagaaagttggtgaaacgtcggcaattttagcggacaggatgctagactcaaaaatgaggatgaaatatttagcatggcgtaatcgaaaattcggttgacaagttaatagtactctattatattcttaatcactgaccgggcagcttcccggataaactctaaaatgcgagaaaactaaaatgggaatcaaacttaatcttcgtaaagtacaaactgcatggttgaacgtattcgaacgcgctaaagaccgtgaaaataatgacggttccattactaaaggtacttataacggtacttttatcctgactccggaacacccgcaaattgaagaacttcgtgataccgtattcgctgtagtgtcggaagcattgggagaggccgccgccgagaaatggatgaagcagaactacggcgaaggtaagcacatggacaagtgcgctgtgcgcgacatcgctgagcgcgataatccgttcgaagacttcccagaaggtttttacttccaggcgaagaacaagcaacaaccattaatcctgacttctgttaagggtgaaaagcaggtagaacctgacttcaatattgatggtgagcagattgaaggtgagcaggtatatagcggttgtgtggctaatatctcaatcgaaatctggttctccgagcaatataaagttttaggcgcaaaactaaacggcatcaaatttgccggtgaaggtaaggcatttggtggttctgcggtttctgccagcgtcgacgacctggaagacgatgaagacgaaacgccacgtcgggaacgtcgtcgtaaccgttaataacttttcactctaatttaggcggcttcggtcgccttttctataagggtcaaatgaatgaacctgctttatcttgatactgaaacattttcagaagccgatttgaaaaaagtcggttcctatgcttacgccgaacatccgactaccgaaattgttatctgcacctacgctttcgatgaaggccctgtgcaagtatgggacgccaccgacggcagcgatatgccgcgtgatttgcgtcgggcgatgctaaagctgcaaaaaccagacagcaatctcaaactggtaggccaaaacttccttatgttcgaccgaccagttattaagcattgctggggattcgaactcctggtagaaaacattatagacactatgatagtcgcgttccgacatgccctcccaggttcactggccgcgctgtgtgaggttttaaacattgacgcaagcatggctaaggataaacgcggtaaggcgctgatacagcgattcagtaagcctacgcccaagaactataagattcgacgttatactgccgatacccacccaaaagagtgggcagaatttattgcatacgcaaaaagcgacattacgtccatgcgtgaagtgtataagaaaatgcctaagtgggggaattctgagttcgaagaccgcgtgcttcatctggaccaggtaattaatgaccgtggatttaaggtcgatgttgccctcgccgagagtgcaattgaagcggtgacgcgccataaagaagagttacaggaagaagcccaacgcaaatatggcggttcactaaccggcaaagacttcctgcctattttacaggaactagcaccggcacaccgcatccacaacgcacagaagtcaacattaaacgacctgctggccgatgaggatttaccggacgacgcccgcatgatcattgaaatgcgtcttggggccgcatccactgcatcaactaaatacgcaccgttgctgttaggtcgttcttccgatgaccgccgtcgaggttgtctgcaatacggcggagcgaagcgaacactgcgatgggccgggaaaggttttcagcctcagaacctggcgcgcggatattatcacgacgatgaacttgataagggtatttccgcattacttaaaggccgtgcacaccgccgttttgatgtggccaagctaacggcatccactgtccgtagctgtataatcccggaagacgggtgtaagtttgttgtcgcggactattccaacgttgaagggcgagggcttgccttcctgtccggggaagaaacagctcttgatactttccgcgcgggtcttgatatatactgcgtaacggcaggaaagatgttcggcatggacccagatgatattaagaaaaactttaaggatatcagacagataggaaaagcttgcgagcttggcctcggctatgagggtggcgtcggagcgttcgttacgttcgccaaaaacttaggtcttaaccttatcgaaatggctaaaacaatggccgggactttccccgaccatatctggaccgctaccgctcgcgggtatgagtgggcgcggatacaggaggcaaaacggccgccgcggccaggtgaaaaggatgaccgcccatcgtatattttggacaagaaagtatggcgtacatgtgatgctattaaacgcatgtggcgggaatcacacccgcaaaccgtagctttctggcgcgaccttaaagacggtatccttgccgcggtacgtaatcccggtcgcgatttctgggcgggcgcaaatatccgaagtaacggtgaaagagctatacgcatatggcggacgacagaaaccgattcatccgggagaaacatcccgggatggtggctatgcatggagctgccgtcggggcgaatcctatcatatccaggaataggcgtcagcgtgacaaaggaaaccgacgaagacggacgggtaaacaccaatgtaaggattaagtaccagggggagaaccagttaacgcggcaatggactaccctgtatacccatggcgggaaggcttgtgagaacatagttcaggcattttgtcgtgatttattggcctacgcaatgcttaatgtcgaagctggggggtatcctatagttctttcggttcatgatgaactggtatgcgagaccccggatacgccggattacacggtagcggaactggaaaagctaatgtgtgcattgccgggatgggccgatggtttccctcttgtagcggaaggtgcggagttaaaacggtatgctaagtaaactaattatctattatttccgtgaagaagattgtcgaatctgcccgcgctgcggtattgaacacacgaaacgcgaggggtgcatagatgagtacgcctgagggcaagattcagaaatacgcaaaagagaggttcgaggccctggggggcctcgtacgcaaattgtcgtacgaaaatagagctggcgctcctgacctgctggtaattctccccggcggcatcatctggttcgtcgaggtgaagaaagacgaaaacacgaagcctgacccgcaccagctacgggagcacgagaggatgcgcaaacgcggtgcgaatgtttttgttgttgggtcgaagaaacaggttgataaattaatagaacactattatatttagttcacaccaaacagaaataaggaattgagaaatgaaatacgaatatgcccgcaaaccagcacgcgacatcgtaccgggcgatatgattttcaacgttaagacacgtagccctgttgccgtagatacagtatttgttgagtcaaacggtaaactggttatcgaagatgtaactggtaacgtcacggcgttcgggcggaaggaattggttctggtggcgaaatgagtaaattcactagaaggccttatcaaaagctcatgacctcgttcatgctgcgacacccgcgctgtaatatctgggcaagtatgggcagcggcaagtgcctgaaacgcgggactgaagttattatgtttgacggtacgaccaagaaagtagaagacgtcatcgtcggtgacgtgcttatgggaccagattcaacgcctagaaatgtactgtctctcggtagaggccgggagatgatgtacgaggttaaaccgcgaaaaggcgagtcgtatacggtaaacgaaagccatatattatcactgcgaactactacaggtatcgcaaaaggttcatggccagataacaccgttttcgacataagcgtccgcgactggcttaagttgccgaaatatgtaaccgggcctaatgggtatcttaagggttggcgggtacctgtcgattttccaaggaaagagcaggacgaggctttattgcccccgtaccttatggggatatggctgggcgacggtacttcgtcgtctggggccataacatcaggagagaacgaaaaggaaataagggcgtatcttgagtcgtacgcagcacgtaatgggatgcaaatacgaaaagaaggacttacatggagtatatctcacggcaacacaggtcataagaagcacgggtttacgcacgcgctgaaatcggcgggtgtcctgaacaataagcatataccgcacaattacaaatgcggcgaccggagacagcgtttagagttgctggcagggcttctggatagtgatggttactgcgatttgtctaaagcaggttttgactggatttcggtaagcgagcgactcgctgatgatttttgctatctatgccgttctcttggtttcgccgcttacaagaagaaaactcgtaagcgttgcgcaaacactgatgtttggggggattacttccgcgtatcggtatcaggcgatttttctgatgtacctttcgtgcgtggccgccaccagaatttaccgaagcgtaatattaataaaaacgtacttaacgttggaatagaatctataaccccggtcggcgtagatgattattacggcttcactatcgacggtgaccaccggtttctattaggcgacttcaccgtaacgcacaacacaagctcgacaatgtgggtgcttaaccggttattccgtaatggtcagcttaatgatgacgaccgcgtgttaatcctcgccccgttgcgcgtcgcgtcgggtacgtggccggcggaacaagagaaatggggtttcccgtgccttcgtgtcatcgatgctacgggttcagagaagcgccgcatagcggcgctcgagtcagacgctaacgtggtgtgcacgaactacgaagttatcgagtggctgattgactactacggcaaagatgactggccttttacggttatcgtggccgatgagagcacgaaactgaaatcattccgtagccgttccggtggcagcaagagagcgaaggcgcttagtaaagtggcgttcggcaaggttaagcgtttcattaacctgaccggtacaccgtcgccgaacggcctaaaagacttatggggccagaactggtttatcgatgccggcgagcgcctggggtcttcgtacacagcattcactgaccgctggtttaactcagtacagaaagggaagtcagctatggcacgagagtaccacgcccgccccggcgcggataacgagattcaccagaagatgaaggatatcagccttaccattgatgccgccgagtggttcggatgtgaagcgccaattattgtgccggttgaaatcgacctgccgaagaaagcgcgtcaagcctacatcgatatggaggaaaagttattcgcggaactggagagcggagaagttgaagcggctaacgccgccgctaaaacggctaagtgcttgcagattgcttccggtgccgtgtatgtgtcagggccggatggtgaagcaacgaaagactgggagaaagtgcacgacgcgaaactcgatgcgttagagtccattgtcgaggagttgcagggtgcgccgctgctggtggcctatcagtttaagcacgaacttgagcgcattcttaggcgattcccccaggcgcaggcgtttgcgaaaggcgctaagggcaataagcagatggaagcgtggaaccgtggtgaaatacctatgctcctgatacacccagcctccgcgggccacggactgtcgttacaacacggcgggcaccacatggcctttatgagtcaggggtggaacctggaacattatttgcaggttatcgagcgtataggcccggtaaggcagatgcaatcaggatacaaacggtctgtattcatttataaaataatagcccgagatactcttgatgaagttgtcgcagcgcgtacagacgagaaaaggtctgtacaggatgaactacttaattatatgaagagacgaggtaagaaatgaacatcatagccccgattccggcattgcaaaaacgtattaaggaactcgaagaagatgttctacgactatgtcagcaaagagacgccgctaatgcgcaacttgactttattatggagaacctgaaatgcgatacactaccggagcaaaatgcttcatagcaggacttattattgccggtgtttttactatgtacgctttgtacaacgtaatttgaagaaggcccctttcggggcctttactatttatgccaaagttaatcttgtatagcttccatcggcccgcttaactaacgccttaaggctatcgccttccagatacagtgatatagccccgttatccctgacagcgttgtctggtagggtggacaccgctaccggtatctcaacagcacgataatctcccctgtttatttttaatgacacggcatcaggcaaggaccccgaaatagcggtaatttcggtccatgccgaaccagaacctggcccgccgttaatgtgggagtacacggacccactatcaagggtttttgacagtttatggatacgaaaccgaagcgccgcagaaccattattgaaactgtttatgcgagagttaccaagaccttcctccatcaaattggctacgttaatccttgaggggtctacgaagccggttataccgctcacagtggagttaggcgcgtctatcgtaaggccctgttcggtagttgaccggatgccgaccaagcgcagcccattaacacggcaagcccctgaaatgtaaatctgattagcaggaaagtctttagtattcgtgtctattaccgcaatgtttgtgaatactgtttcatgcgtcaacagatatgctccactgccagcgcaatcttctacggttatgttagaaacgtatagcccttgcccatccatgtcgaagccaactcctaaacaccctctaaccaacaagttatcaatcaaatggtttaacgggagcatatgtactgggtactgagatacagagtaatcccccggcctatctgcttccgggttcatgtcggtatctgcgcctaaatcgaatccgtcccataccgggtacaaaactacggaatcgcggaattgaagattataatttcgtgacgttgtggaacctacagtcccctgccatgtcttaactccgctctcgccggcacggtatgaggtaaacccaataaccccaccatcacgttcaaagccaccattattacgtaaaaactgggcgctacttaccgaaccgtaacttgtacgcccgccaatgacatagtttcctttaccccaatccccgctcaggttttcaaaggtaattacgccgtcttttccgccgctcgggttgtcagcgtctaccatcttacagaaatggcatccgcggaacaggaaacacgccataagaccactcgcccggtgaacctcgacgcctgtacattcccgtatctcaagaacagacgaaatgttctggtctttagcttccggagggagaagggattctatacccgggaacttggcgtaatcgtttaccgtaggctggtatccatctgttttagactgtttaagtgtggccacgattgctgcggggtcggttatccactgattatcgtcagtccacggtttaatcacccacggcgttgtgacactctccatgtaagcaccaactacaacggaccctctaccaagttgtgtgaaaacaaggttaccgtcgccaataaacttcgctttacagtcgatggttagggtcttaccgccgaagttaacggtttcgttattgctgaaattgtagtcacggtcgataagaacgctgtcgacggcagcatcagctaattgctgtaaagtggtaaaatccgacaacttaacggagtatttaaatttcttatccgcttctgcgcgaaaggtgatatccccgaacgcgacccaagcggaatccgaaatgccacctgtcgtaagaggtgtcgacgccgcagggataactttagggagagacccgcgccatgcgtaatagttaccgtctccgccgtcttcaataggccaaagaaccgctttatccgcgtcgttaacccctagcgtcccgccggttgtgaagttgaaagatgctggggagaaaccagcatcacgtaaaaccgcgggcagggtcttctgcgtctgcccggttacctggttagtagcgtagtcgatatctgcgccacccgctacaccaccctgtttgccggttatcacctcggcttcgaaaatctggtgttttttagctacctgtaaatcgttaagtgacaatacatcaccgcaaccactagacatatgactatcctcttagttaaaaccgttatcgaatccgttagagaacgcacgaccgaaaggcggtacgttgtcgaacttatagaaatccttgtcatagttaaacccggttattttgactgttctgtcatcgccggggtctactgtagaaacaagaatcatctgagcattatgcctcgcttcgttgccgaatgaaaattcagttttcagggcgctattacccgtataaatggcttcctgcggtacagaagtcataattacttgtcggtcatgcgcaccagggactacacgaacactttggacgccaccgtctctcagtttaagaaccaaagaatggtcatccccggaggtgaacttaactggctgtgacagctccacggtgaggccgttcaccgcagttacgtagccgtcgtaggtagccatacgggaacccttaaccacactaacggcacgatttgggagggcaaagatcccttcttcagtggcgctaaaagacactgaaattttctttaaaatgttcttctggtggcgacggttcgctgcccagaaagcctgcttatagttgcgaataccttttgaatcgtaggtctccgttttaaccccaccagtttctggtattgttattgtttctttaacgtttgttttagggtctatgtatgagaatttaagactatcgaacacctgagcatcgttaaaggttctggtccatttttcagaagtaccggccttgcttcggtgggtgaacaccatctcggggcccatgcgaggtctttcaaaatcaagaaggatatccgctcctctccggtatggggtgcagaaaatagcgtccgctatagtactaactatgtcttgcatagtggttttgtagtcatcgaacgtgtagcaaaattcccctgcttgcttgtcaccgaaataatcttcaatctctttctgaacggcaagtaacttatccatgtttcgcactgtcaggtttaagccaccaacatctgggtcacgtgccaggcggataagagattgcacggcctgggtattaggggtcatcgtgtcttcgaaaacaccattaccgaggtatttgtacaccatttcggtggcaatcatacgtaactgtggttgcttaacctccgcagcgcgcggcgtctgcttgcgcatcgagtgtacggttgttctgttgccgtagtgcggagtgttatcacgtgtttgtccgtataagttaacgtacgttatttcatcaaccacagacccctcgaaattaaagtcgaggtctgtcacccttctggcgcgcacccgcacccgcgacacaaccggcaggtcggcgtaaatagtgacaccgttgtagtctggggttcgcccggacacggttccttgtgcagtgtatatcggcccataaggggtgctattttcatcaagcagctggtactgtatctccgcggtaaccgacgctagtgttttagatttgccgttgtctttatacataccattggcggcggcgatattggcaactatgcgctcaacctcggtacggtttattgacacccaatcggttaaggttttctcgtatgtgttatccggttgcagcgacgcctctccgtcacctcgaaaaaatgaacctggtcttatttcttgccaccgaccgataaggccgccaggtacaataaatgtaattgtcccttcggatacgcttaacacttggtacccacccccggacaagtcgacgtctgtatttgtccacgctgacaccaacgaaaacgtgtcattaggtaccaacactccagaaaaatccgaaccacctgttgggtcatatattgtgccaatgttcccggacaaagacgggtacccggacatgtaagagaaagaaatacccaggccgttgggagctttaagaaccacgccatcgacttcgttagattctacggtaatgtacaggccctgctctatagggtcgccgaccatgacctggggtgccgatgtgttattgggcgatgtatatggtgagtacacggcaaccgacgtgcctgttatgtctgatacgcgggtgtccccgtcagttataccttcagggtgtatgtctaggtaaccacgcccggcgtcgtaatagccgtactctacaattttaccagcggcgttaaacaccttataagtagacataaggttatttgggatagtttgcaccgttccgcagatgtcgtagctacgctcgtatgggcgggccttgttgttacggtcggtgagactgttgttcggggaatcagcctgcgagttcgcaaggttagatgacgcccctttcacggatggtgataatagtttcgctagcggtttaaggacaacgctaaaaatcttcatgacgcctttaatagcgccaccaccggcagattccacaatgtggaaagtcgcgttttctttcagtgcttcgaagtcttctgttacgtcgttatcttctccgatttcgtcaatgaaaacacgtaccggaaccccatccggaacatggttcacgacgaagttcattggattatcgtgataacgtttaacgtcaaatgtgccgtcttcgttgcgggtatagtggattactagcgccaaaattcaatctccgaataagtatctttaaggtcttccagactatccagtctaacctgtctggacgccagctcacagtggcttaccattccttcgtaatataccccagcatgccacactattcgccccctatgtttcactcctagtagaaccgcgtcaaaattctgcggtgtgaccgctcgtaccagacctttcggattagagtggccatcgtcgaaggcggcgtctattgccgttgggcttgttacatcgaacatcggggtcgataaccccgcgtcagcccggacgttgcggacgtggtgccaacaattacggcggcggaagtcatacggcaggccagtgtaatcgttaatattcatgacgtcagtatcccgcggagtaagggtatttcttccggcgtcatcaggattccggtagctcgttggttaagcatgggtgtacctgtttccgctgtgaacactcctttctcttgcgtgagtgtttgcaattcatataccaccggcccatcgcatgggtatgacaggtccgtgctcacgtaacgccggaagatgaatttgggcaattccttattatcataagggatgcggcccatttcctcgtccagtatattaagcacatccggcaaagagaaagaggcggtctggtccatgtcgttattgttggcggcgttcttggcctccatcggcgtaccctcgaatgtaacaacctcacccgtctctagagtagcggtcaagtcgttagccccgcgcacaagcaaccagcgcttcgaaagaagagggtgggatatctccagggtaataaaatccatctcgccatcgggattggacgccagcttacgccggtatgccgcttctactgattcctgactcattgcattgggtcccaaattcgagggaatgtagtttggtacactccgtaagtttttagaaattcgccaagacaatcgccgtagcatccatataagtcgggcagattctgggtaagacaggagctgcctacttcctggaatggcgatttctcggctgtggccgtgaaagttattacccagttcttcccgtcgtcggtggactcgttccacgtagatgttagagtaacctggtaatcctcgataccctgacccaagtcgtgtttcatccagaaactggaagcccctccgtctaccttctccatgaaactgaggaatgcttgtctacccagtggcgacacgaccagcgtaacgctaatcgggaacacatcaaagtatgtatcacgcccctgtcgaaccccgccaccggacaggtctactcgccacacgttgttacctcgcgtcatcgagtagcctttcgatactataggcctcaaagaggccgggaaatggtaatcgctcatatcaatatcctggttggccgcgtgtagcgcgacgtgatttagaaatggcgctgttactatcctgtagcgcagaacttacggtttcactgattataatacgtaaacgaccttcgtcgtcgcgttctgttgcagccgaatcaattcttccagtggtgttgttcacgatagtaacattatctccaccggatttagcgccgttctcacccataatctgtcgcatctgctccgcagtgcggacacgtgacgcgctagctggcatgattacttccggcttaccgcgttcagcgatagtagacatctgccctgcggcgaggttaccgccctgctcgcgcgcggagcgaatcttacccacgttggcgagacctgcggctacggcagcagcagcggcaaccggtgctaaatatggccctactactgggatggccgccgttgatttatacgcttctactgctgacgtgtacgtagcgattgtcgcctgtgcgatggcaaatgccttgtacgcggtggacgcttcccccagtgctgacccgatgttgctcgccatgctaccgaaagccgcgccggttgccttcgcccggtcaagggagtaagactcatccatagcgcttaacgtttgctggtaggtttcttcgccaatcaaaccctgcgcatgaaaatcgttaagtttttgtagcttgacctcgtactggcggtctaactccgcgaactctccttcagcaatggcctgcatctgagcgacatacgcatctgcggagaattgtttttccaggcgctctttctcgcgtttatctaactcagcctggcgggccgtatccgcctcgagcataatctgcgtcttagcggtttcgtattgctggtcactaaggagacggttgctgtagaactcctgtagcttagtaagttgctcttcttggttagcttgtatcttctggaatacgtcgtcgttaaggtggattgactgataatagaaatcctcagcggctttcttctgacgttcgaatgcgtcgacggatttcttagcagcattgtccgcggaatcgcctacagaaccgttgtctttacccgacactttaaacttaccaagaccctcggccttggccgcttctttatcgatgtcgtaagccgcgcgcttcaatgcgataaggtcttcctgagcttctacctgttcacggaaacttttacgtgtcaggtcagtcacagctctttgggtatccatagcaagcgctaactggaagttaaggttcttggcctcagctaccgactctcccggcgccaatgcggcctttacaagacgtcctgcatcctgagcgcctttaaccatatcttcaaaaaaccgctccattgccgtaaattcctgtgcaatgaagtccagcgcatcagccgctgattcggctattgttgacgcggtctcctcaccacttccggtaatgctttcactagtgtaagtccaggcggagttcaaagcgtttaacgcgtctgcaaccgtcccaaacgccgcgtcgaatgaagcctctatcgcacccccaacatcggatgccatgtcaccccacatctgcatctcggctacgaactcaccggatgcgacctgagcgtttatctcctgtatcaggtcgtcaatataacgtaaaggttccgccagcgatcctacatccagaccagtagcgagagtcatctctaactgggaccatgagtcctccgcactagcgatagccccgttcagggtattagcctggtcagccatcgccccggcgaagttaacgttgcctatgttgaggagatatcgctcaatatcggcagcgtttttcttaactacagtagtcgtcccctggaaggtaaattcgatatcactttggttctgcttggctttaatgccgaattctttcagacgttcgaattcaaaggtgctcgcatccgcgacggcctcaatcatctggttaaggtctttacccatcgcggatgcagtattgccgtaagagcggagcgcttcttctgatggagtgaggcctagagctaccagtttacggaacccttccactgcttgttcaaggccgtaaggtgtgtcacgggcgaagtcctgtaaaatactcagggcctgttttgcgccttgagtactgcctgtgagcgttttcaggctagccgacatcttatccagttgccgttgcgactcgactagttcctgtgccgccttataggccgcagcggctacagcggcagccagtccggtgaccgcggcccccgcaacttttgcagatttagagaggttatcaagacggtctgatgtggccttcgcgccttgttcggttacctttactactaagctagctacatcagccatcgtttctaccctcaaaaatggcgtctaagcccatgataatttcagattcaaacatccctatattctgacctgaaatagtgctatacgccactaaatctgaccaactaagttgctctcttgggtatagctttacgccgtcgtcatcaacgcggcgggtgaatttaacatcacgatacttctcaaaagtggtgagtagttcaggggggcattcaggcccggcgtcctgcgttgtttctcccgcgtcttttattacccccatagcgataagcgcggctttgtgcccatcggcgatactgtcaaacttgcgcctttcgtggcgggttatgaagttccaacgggcaaactgaaacaacgcgtctacttttcctgcaattgacgcaactgttcgtggtggaatacgactacatgttccgccaggcgcttatattgggtgagaagagtcttaagattttccttagtgaactcatcatcgagactccagccattcaccaattcgagagccaattgtcggtttaggtctcctgccgcgtcttccatttttaaattgtattctgagaagtctttttgctcttcgcatttatcccgaagcggctttaatttacctaccgccgctcggtacgccagggtgaaggcacgcatagctttaaccgcgacatcggcctccggggagacaacgttcaaccattctcctgaatctgtaccatcttttaaagggatcggcatgcgcgcgcctttctcggcttcggcttcgtagtaaaaatcgctaagtttcatagtaatgccctttggttagtcggttactggttggtagttgcaccaggcgggaaccacccgcttttccggtgcgaccgtagacgccaaataagaataacaaaatagcttgcataaccgttaatagtgtcctattatctctacgtcaactaaacaatatgaggttaagaacatgagcgtttttatcggtattatcgcgtttatctatttcattccgttcctggtagccttgctacgtaatcataaagcgaaattaggaatattcgtatgcaatcttttactggggtggattcttctgccgtgggttggggctctcatctgggcctgtaactctaatgttaatggtaaataatctggcttaatcaaaattacattattagaatcaggtttgtcttaagcaatcggggggggg

SEQ ID NO: 5 - DNA, 51608 bp, unidentified
  source 1..51608: mol_type = genomic DNA
cgctttatgacgctatcgccaaacctggcaaactatgcgccggaagaatacgcaaccgtagcaaaagtaatgcaagagcttcatgcacttgtgaaaatcaactttgtcaacaatgaggatgaataaatggaaaactttgatttagaagcattaatggcaacaatcgacacgacaagcgcaacgcagtttaacgttgtgcgccttataaacctggatgcagcagaaataacgcttgaccaattcaaagaaatgttaagccaggctaagagcatttatgaatcagaagaagatcacgaaatcaccgttatctgcgtacagccgcgagaggatgaaaacacattcctttacgcctttgtgaatgagggtaatgcataatgctttgtaagatccgccttcatggcaacaatgagctaaaagcataccgcataattgcaagcgtcattatcggtcgcctatatctaagatgtggcggcagtatggtaaagctgaaatatcggcctggtagggtcatcaattctaatactggaagatggattcaaaacgatgaattactatggcacaacgcaaaaggccgcttgtatatcaagacactggaggatttgcgagaatgaaaacattcgcatttgtcgctgcaatgtggtgcgttgcaatcggtcatcctggcgtggcgcttgttgttcttgtcttgatattgttgggggcgtttgatgaataagaaaattattggcgtttgcttggcggctttgttcctgattggttgtgatatggagtcaaccaggacagacagcgagcaagatataaagatgcagattggaggcgagtcggatcgcgttagtgtagttaagataactgaattcaaagatgcacttgcttatgatgattatcgcgggatttacataatcacagataagaaaactggcaaggaatatattggtgtatccggcattggcataacagaaaccggatcgcatggttgcggcaaaggatgcacaaggcaagatgaaagatagcactttttgttaaaactgaatcggggtagtttggcataattaccccatcgaaacacaacggagaaaataaaatgcaattcgaactaacccgcgtaactgttgaaattctgaatgacggaacctatgacggaatgaaacatttaacattcccaatccgaattgagaattgccttgattatggcgtggaagtaggaatggttgacgttcctcttgatgccctggttgcggctggttatgacaaggtaaaaggccgcatgtgtgaaattcctgattgcatggatagtgacacgtttttaccgttcagctatggcgaatgccgcattgttaagtaaggagcaataaatgagcagttggcacaatgagcacgtaatgcaattctaccgacaaagaataaagaaaatgctcaatagcactaattgctaaaacataatcctggggttgcggtatagtaaccccacaccaaacgaaagaaggaaatcaaaatgaaattagtacgcaacgcaatttatggtttacacatcaaaggcggtaaaggttcaatatatgttcacctttttcatgtgaatgagcgcaaccaggttgatattgcgatgtatacagatcagtttctggtaaaaggtgaaattggcgataggtttaatccgcaattcgtagaacaaaatccaacgtatagccatgaccattgggtttgtgatgattactataatttcatcatggttgatagtcacgttacggacacgcggaaaataacagttagcgactataagcgcatgactcgaaaagtattgaagcataaagcgcaatagcacaaaatgttaagcggtcatgtgtcctgatgggtataatgaccgcatcaaccaacaaaaggagaagtaaaaatgtctaaagagtctatcaagcagtttctgaaatacggttctatgattgtgcttgctaccgttgtgctggctggcatcgcgtatagcattattgagtttgcggggtaattgatgtttacgatcaacttgtggggaagggattatgttaatccctatgttgtgacaaaacagcgtttcgttattttcgacctggacggcacattgtcaaatggtgagcaccgcttgcacctgttgccaacggttgatttgcaccttactgaaagctggagtgagtttaatcgcgccgcaaaggatgattcaccattcagcaataacatttccgtgtgcaacgccatgtttgatgccggatacctggttatcatcctaacaggccgtagcgatgaagtagaggcggaaacacgcgcttggttagctgaaaacggcgtttctttcgattggctgattatgcgccgcgccagcgataaccggaaagataccgtaatcaaagaggaagttttacggactatcggacttgatcgcattgtcgcatgttgggatgactcaccaaacgttattgcacacctgcgcggcctgggattgacggtttaccaggttattgactacggcgagcagttacacgatcacttaaagtctcacggagttgaaaagttatgaaactaaaatgcatcggtgaatatccaggctttacggttggcaacatgtacaatgcaacaatcggaatggatcacacatttggttatgaatgctatgttgtcattgatgatgatggtgattcacgtagcattgatttcgaaacagaggattttgtagaggatcgctatctgtgattgaaaactatgatccaatggcgcttgcctggggaatttgtgcttttatcgtggcggttgtgatttgcttatgggatatcaacaggcgtgacagatagcactttttgctaaaacttgctaaggcacatttgataaagtgtgccttgtcgagacaagcaaggataatgacgaatgaaacattacaggtgcggcagtcttatttatcgtgacgatggggttaatatttttgttgcctattatgacaagatggaattttttgaatccatttacacaaggcgcgtttttaattcaatggttgatgctggatttatgaaagagtgtgagccaccaaaagcgaagccgttttataaaaatgttgatttctggcataacgccgtattaatcacaattctgttaattctttctatggggtttattttcttatgacacttgataataaagttgaaacacaaaagacgatcaactatctgcgagtgcttgctgatgctcttgagtccggcgaggctgcattaaatgatgttagtattgagtccagaaatcacggatggactcagatcgaagaaacattaacagttcaatatttcaaggtaaataaagatggctaaaattatcatccttaatgcgccgcctggcgctggaaaagacacaatagggaaactggttgagaagcattcaccaattcatgtgcgcatgattagctttaaacaaccaatgtttgagattgcattagcaatcctgggcgctaaacgttatggtgatttcatccttgcttataacgacagggagcagaaagaaaagccgcatgatttcctgatgggtatgacttgccgccagttcatgatctggattagcgaggaagtaattaagcctaaatttggtgatgattactttggcaagcgctttgatgaaatcgcaaaagaaagtgattaccctgtaatttgcactgacggcggttttcctgatgaagtgatttcgcttattgaggctggtcatgatgtgaagttgtgccgcctacaccgtcgcggctttggctttgatggtgatagccgtaattacattcgattgccaatgaagatgcaccgcagaaatatttattgcgagcacgatttttatttgactgataatcaaccaatggtaactgtttggacaattatcaacatgtatctcaaatagcacgaattgctaaaacttgcgcaaggccatttgatatagtggccttattcaagagaagcaaccaatcgggggttaaaatggctaagttaacagtgacgatgaaattcaccaaagcagtttgcgaatgcgattcgtacaaatacacaaaaggatcatctttcggtactcttgttacttatgataaagacggaagccagcttgaagtgattacttttggtaacggtattggcggtgataatccatcattcaaatttgaggaataatcgatgaaaatcaaatgcacaatgtcaaatacggacgcgcttacagtcgggaaaatttacgatgtaacgccagcggaggaaggatttagcccaaccgttaagcaagacaacaaagggttttattatctcaatggcaaagataatctttctattctggtggttaaggctgtaattgcagaatttgaggtggtggaatgattaggcacttaattatttttgctatggtacttttaggtacaatgcttattgataacgacacatgggctactgcaattggaggttgtttgctttttgttgctggcggcatgtttggtgagaatgaggcaaaaaaggaaaataaactttccgatcacttacgcaaggagactaaacgatgaacagaaaaatgcttattcgcgcctttgctaagattgctaaaaggtggggagactctaacaaatcgccttacacatttagccgctacggttataaaaacgctcgcgcatggtgtcgctgcattgcggataactacaacgccgaaatgatggaagattttgcaaagtggattgatacgcctttgaaggattggtgtcaatttgactatgactatttcgcagaagaagaaatttcatgctggtaaggagagtaaacatgatcgctaaaaatgacctttgcaacctgttaacggctggtaatgattatcaggtttttaatagcacagaagaccgttattttattgagtgtgacaatggggatcgaatgtggattaaaaaaggatttttcgaaagtgagcatctatgcaacgcaaagcggatcgcagatgaagcaagcgcattagcagagcgtgaacgaattcgcgaaatagtagcggagcaggttagaaattcatgcttacctggtggcgatatctttaaatcaatgcgcggtgaaatgacgattggaattcaagatcgcgtaattgcaaaatcagttgattctgtgattgagaatgcgcgattcaatgcgttgattaactcagagactaacgaatacattaaagtgttagagcaactgcgtgaagtgctgcgagtgccggaaggtgaaaatattgtcactcatgcaaaagttgtaagagcgttggctgatgcgctgattggcctgcaaaaatagcactttttgttaaaacttccgcgagggcatttgttaaagtgtcctcattgaagcgaaacacggagatttaggaaatggctgtatacactggcaaaatgtttgaaagcgaaaacccgatttacggcggcacttatgagctaatcattgatgaagatcgtgtaatcctggctgacgacgataaccaggaatattacgaagtagaagccgataaatgggataacgacgttatgtttcttgctgataatagccgcatcaagttttatgcagtggaggaagaagaatgatcacaattaacctatcagaagaacaagcgaaatcattgttgcgcggcctgggctggcgaatgggtagcggtagcgaggcggttgtaaacatcaaagaggaagtagcaaaagagctttttgcacagcttgagcataaactgatgccagcctcaacaagcgcggcggagttggcagtgtggcggcatgaaaacggccttaatgaaatggacattgagaagtggaagcgtaaaaacgaggtgatgcaatgatttatattcatagttacggcgtcggcaagtttggcagcaagcagatccgaaacatttacgacacgcttgatgaagcaatggcacagcaacgcgttttaggtggagtggttcaggcgtttgagtcagtggaaaacttagaaagcgaatggatggagcgcgagcacataaaggatctgattgttgatgaggtgtcatgcttgattgacgatctcacaactgacgcgccagggtcgccgcgaaactatgagccaggctcaacgcaatgggattgctggaaggtcaaaacgctaggcaacatccgtgatattatggatggaaaatttaacgaaattgaatgagttttaaagcgcctcaatcgcattctaagaggcgctaattttggaaaggtgattgtatgcctaactacataaaaccgtcgcagtggtgcgcacagaagcaagaggaagctttagagcgcggcgacactgacacagcattgcactactttgaaatgtaccaactttggaaatcaagaggactgtaaaatgtttggacttaatgagactgaatacaacattgttaaacgtgctgcaaaagagtgcatgaaggatctgaaagtcgaggtgacaagcggaaacaaatacgatcatattgcggctggaattatcaccaaacatcacactccgatttcaacgctaatcacgcgcacaaagtttgtatggcttgctggctatattgctggtcgatggggtaagcctggcgagtatgaataatagccagttggttcaagtaaggttgacggcgtgatttctgattggtagattaaacgccgtgaacataagatgtgttttcacacaacgcgtggcgcgatagccacaaaaccaaatcgggcagggagagcgcaacgcgccccgatgccagcgggaaccgtaacccgcaccattcaggagaataaaatgagcaataaagaaattgcaaaggttacaatcattgttgagcgtgacggcaagaaaacgcgatacaaaaaacgctttgcaaaaggcgaggcggtattaggccggatcggtgatttcatggtgaaattgcaagaggataagccggatggtgttcaaagttaagttaactatcaggcgcatgggtcgcaattgcggatcatgtaagcaggattttgaatgcgaggttgaagcgctaaacgatgttgacgcaattcagatcgcaaaggttctttctggtgctaaccctgaaacgcaccaattttcaatcaactatgtgagggctatacaatgctgatgtttgcttttggttttgctgttggttttgctgttgctgcatttattgcaatttgtttttatggctttgttaagatcggcgtccgagcttgcttgaatgacaatgagcttgcacttgcgatctataaaaacaaagatgatgtttggaagattacacgaaactttgaaaagattgctgatgaaattgcatttcgtcgcaagtttggcaagcctggaagcatcaagtacattgactgatgcaatgccaaaagataacccgccttgcgcgggtttttttgtgcctacaatttagctataatcacgcctcaacaggaggattttattatggctaaaaaaacagaagcgccagcggcggaaagtctaaactttaaaaagttatggaataagcgatattccgatctggtaggtagtgagattgataaacgcgcaactctaacgccggaacaggtcatgaatctgattgttcagtattttgagtgggcggagctaaacgccattaaagcaggtgaaacggcatcctttcagggtcgagtgtatcaggacacgattcacaagccgcgtatttttacatgggaagggttaaagctgttttgcggtttttccggcacagcactagcaaagtggaaacttaaaccaggctatgatgttgttattgagtttgcagaatcggtaatcaaagaacaaaaataccagttagctgcaaacggaatgattaacagcaccatgatcgcaaaagagcttggcattgataaaggcgacacgttcaatattagcgccaacgccaaagagcaagacaatacagaagaagcgatgaaaaacgcagtagaaagcgtcctaagcaagatttaaaggtgaatgctatgttgatttgggaagatttaacaaaacttgaaaagctggctattaaggaattaagcacacacgattttgatactttcgttaaaatctggtttccgatccagcagggcgaaaaatggattcctaactggcatcacctttatattgcaagggcaattgatgaaattattgaaggtgtgcgcaaagataccatttttaacgtaacgccaggttcaggtaaaaccgaattactttcgattcactttccgccttattcatatctaaaattgaacaaggtgcgaaatctgaatatttcgtttgctgatacactggttaaacgtaacagcaagcgtgtgcgagatctggttaactcgcgagaatggcaagagctttaccccgcaaaaactggtacaagcaaggatgatgaatttcagatccttaatgacgctggcaaagttcgcctagaaatgattagtaaatcaatgggcggtcagattactggtagccgtggcggttacatcacgcctggcgtttattccggttgcgtaacgcttgacgatccagaaaagccagatgatatgttttcgaaggtgaagcgtgagcgcggtcaaatgatcgcaaagaacaccattcgatcacgtcgcgcacattcagaaacgccgattatcgttattcagcagcgtctacacgcgcaggatatgacctggtttctgatgaatggcggaatgggtattgaatttgatcagatatcaatcccggcgatggttacagaggaatacggtaaatcgcttcctgattggttgcaaccgcactttgaaaaggatgtgctttcgtctgaatacatcgttatcgacggcgtgaagtattattctttttggcctagcaaggaaagcattcatgacctgaaagcgctacgcgatgcggatttatacacgttcctttctcagtatcagcaagagcctattgctcttggcggtaacgcaattaacgttggatggttccagtattacggaaccggagaaaaatcaacgatgccaaaaccggatcgctttgattatacgtttattacagcagataccgctcagaaagaaggtgagctaaacgactacagcgttttatgttattggggaatgttcaaaggccggatctattttattgacggtgtgcgcggaaaatgggaagcgccaatgttagaaacgcagtttaaagcgtttgttaaacagtgctggaaccgtaacaaagaatgcggaaacttgcgtaaaatttacgtggaagataaagcgagcggcacaggtttaatccagaattgccgcaaagcatttccgatagaaattacgccagtgcaacgcgataaagacaaagtaacacgctgcatggacgcacagccagtgatcaagaatggttatgttgtattgcctgaatcgcatcacatgcttgctgaatttctggcagaggcggcggcattcacttatgatgatagccatcctcatgatgatattatggataacctttttgatgcggttaatattgagcttaaccttgctgataatgctgttgatagaatgaagcgtttagctggattagcgaagcgttaatgtgtaaaatcaaaggctggaatattccggccttttttattatgggggtatttatgagcgacaaggtaaaggccattgttaaagaagatggttacaacgaaattttcggatctaaagatggaacgtttaggccaaatgctttttacatgcaaagtgcggcatttaaaacgttatcgcaattttatgaagaagatggaatggcgcggcgcattgttgatgttattcctgaagaaatggtaacgcctggttttaaagttgacggcgtgaaaaatgaaaaggcttttaaatcgcgttgggatgaattgcgattaaacgctaaaattattgatgcactttcatggtcgcgtttatttggaggttctgcaattcttgctgttgtggctgataacagaatgttgaaatcacctgttaagcctggcgcacaactagaagatattcgcgtatatgatcgctatcagatcaccattcaggaacgtgaaactaatgcgcggagtgttcgttacggtgagccaaaactttataagatctcacctggcggagatataccggaattctttgttcattactcgcgtatctgcatcattgatggtgagcgcgtatcgaatgaaaaacgccgtcaaaatgatggttggggtgcaagcattttaaataagcgcttgattgaggctattgttgattataactattgccaggaattagcaacgcaattgctacgcagaaaacaacaagcagtatggaaagcgcgtgatctggctttgatgtgcgacgatgaagaagggcgctatgcggcgcgtttgcgtttggctcaggttgatgacgaaagcggagttggtaaggcaattggtattgatgccaccgatgaagaatatgaggttctcaattctgacgtttccggcgttccagagttcttacaggaaaagattgatcgaattgttgcactaactggcattcatgaaatcatcctcaaaaataagaacactggcggcgtttcagcgagtcaaaacacggcgctagagactttctataagttgattgatcgcaagcgcggagaggattacaagccgattcttgaattccttttgcctttcatgatcgcagaaacggaatggtctattgaattcgaaccgttaagcgtgccgagcgataaggacaaggctgaaattatggctaagaacgttgaatctgttgtaaaattgaaagctgaacaggcgattgatctcaaagaaacaagagacactttgcgatccatttgtccagatcttaaaatcagggacaatgacaacattgaattgccagagccggaagatttagaaccagagccagggcaagagggaggattgaataaatgatgtttcccgatatgacagagagcgacgttgaaaaggcgcttaatgatctggcggttgatatcactgaaaacaatgctgatgtagaggcggcaattgccgcctttatgttgctggtttacgggttcgccgttagtgcatacaaatatcacactcgccaatttatcagaatggcaaagcaaacaggcggcgcaaataatcgatccgttatccttctgattggtggcgtttacggaaatatgaatgaacgttggttcaattcaaaatacataacctggcgatcacttattgaggcatccgtaaggaaatttgcgtataatgtgcttgatgattgggaagtacaacagcgtttaaatgagttacgcgagcaagcagaaacaagagcacagatccgaaagcgcctaagaaaatataaggcgtgggctaaaaatagggcgtctggttttgttggttcgtggtattccgtgttaatgtatcaacgcgtagttgatgccggagttacccattacatttggcgtacccaggaagataatagggttagacctgaacacgcgaaattagacatgaaacggatctcattggcaataaagcacatattcccagggcaggaatataattgtcgctgttgggcgcaaccggattgggaaacagagcaagaggtatatttatgaagaaggtgcaacgttttgatagtgtcagagtaaaggcgcattttgatgaacacggttttttagtggatcgtcctattgtggcgcgaattggtttgcagatttatcaaacgccatacggcgagcgtagagaattcaggccagcaagcgaagtgtttaaagctgatagcctggcgacttacgcagggaaaccgatcacagtaggtcacgtaactgtaacgcctgaaaacgccgaaaaggttgttgttggatcgtgttccggcgctggcattccaaatggtgtgggagttgaagcgccgttaaacatttacgcaaaacgagcaatcgaaagcgccaagaaaaagcaaacggctgaaattagcgttggttatacgtcgattgatattgataagcctggttatggctctaatgaaactggtgaatacatctttgaagaagatctaaaagaagatgaacagccgccagaaggatgggtaaaatttgatgcactgcaaacaagcattagcgttaaccatattgcgttagtgtttaaggggcgcgctggcattgcaaaattgaatcttgatagcgagcaagaattcccgtatgatatcgacgttaaattaaataaagaggacgaagttatgaccgttaaaattaaactcgatggcgctgttgaatttgacgtgccaaaagaagttgctacttttatcgaaaccgtgaaagcagacgctacagcggcaaaaactaaagccgatagcctggaagcagagcgcgatgcgctacaagcgaaagttgacggcattccggcacagattgaagaagccgtgaaaaaagcaaaagctgatgctgatgaacatgccgcgctggtagctaccgcttccgaagttggagtaaaatgcgacggcctaaccgccaaagaaattaagatcgcatatgtgaaagaggtcatgggtaatgacattagcgcaaaggctgatgcatatattgataccgcttttgatatcgcgaaagaatctgataaaatggcggctcaacgtatcgcattaaaaggcgatgcagcaaaaaacaaacaggatgcagcggaaacagttcttgatccttccgcacgtctgaataaagctaagtaaggagaaaatattatggctcagatttcagcaagttatcatgttgcaatggctcgcgccttgcctggtcaagtttctgacacttccgcgtataacatcgacggcgcttgtgttctggaagaaggcgacattctggttggcgttgccgttcaggtaaaatcagttgatccgatgggtcacaaggtcatgaaacctatggctggcggcggcactccttacggcgttgcaatccgttcccatttccaaaccacatctaaagatggtcgaatggtttatgaggctggcggcggtattaacgtgatgactaccggacgcgtttggatggtgtcagaagatactcaggcgcaaactttcggcgcacctgttaagctaactgaacagggcgttgttaagtctgatggtactgttgttaccggatggacttacaccggagattacaccacgtttggcgatctcaaactgtcagaggtgcaattgcatcaactctaattaataagtaaaataaaagccgttctttttgagcggcttttttattattggagattagattatggctgattatgcaaacattattggagttcaagcgccaggcgcattaggtgattcaagtctttacaatattgatggttgttgctttacggctgatgatcacgatggtgtattggttggcaaaattgtaacggttaagtcggtcactgatgggtataaagaaatcacggatgtgttttccgctaatacactagcttacggcgttgctttccgcactcatattaatacaaatatggatgatgaaggttacatggtttatcttcctggcgatccgattaatgttatttcaaagggtcgagcatgggtattaagtcagaccgttgacatgcaacctaatttcggtgatccggtaaaggttggcgctgatggattcgcggtaaaggatggttttgaaatcgctggctggcattatacaggcggttggcaaaagtggaattcactgttttatattgttgagatccagattaatcaaagtgcgccatatattgttgatggtgaaaagaggcttgttaatggcgcggtattaactcctaatttgcctagcccacaagcgccaaacaaagtaatcacaattactgttgaagtttcaccaaaagatgcagacgataaaacaggtagttggcatgttgataatgatcagtgtgagatcataccgatcagcgacactcaagcgcgtttatcaactaaaaacgatgggccaaatgatgtgcatgttacttgggttgcaaatgacggcggcggagttcagtctatgattgaatatactttcatcaacgccgtaccataaaaaacacttgaacgtttaacaaaaaatgctatattaaagccgttattatgacggcttttctatgagggaaaatattatgtctaaagaaatgaaatatgatgaatttgaagcaaacgtgatcgccaaccatatgcagttacgcggcgctaaaaacgacgcatctgatatgggtatttggacggctcaagagcttcataaaattaaggctcaagcctatgaaaaagaatatccggcaggttccgcactgcgtgtattccctgtaacgtccgagctttctgatacagataaaacctttgaatatcagactttcgacaaagtaggacacgcaaaaatcatcgctgattacactgatgatctgccaactgttgacgcgctgatgacttctgaattcggtaaagtgttccgcctgggtaatgcgttcctgatttccattgacgaaatcaaagcaggtcaacgcactggcaagagcctttccactcgcaaggccaacgccgcacagaatgcgcatgatcagcttgtaaaccacctggttttcaaaggttctaaaccgcataaaattatcagcgtgtttgatcatccaaacctgacaactatcaactccgcaggttggaacaacgccgcaggcactggtaagaagccggaaaccgcacaggatgaattagagcaagcgattgaaaaaatcgaaacgctgaccaacgggcagcaccgcgctaatatgatcctgattccgccgtcaatgcgtaaagttctgatggttcgtatgccggaaaccaccatgagctatcttgattatttcaagcagcagaatggcggtatcactattgaatccatttccgagcttgaagatatcgacggcgcagggactaaagcggcgctggtttacgaaaaagatccaatgaacatgagcattgagattccagaagcgtttaacatgctgaccgcgcaacctaaagacctgcatttcaaagtgccttgcacctctaaatgtactggcctgacgatttaccgtccgttgacgctggttctgattaaaggtcttgttagcggctgataattagattaatatagggaaccgttaaaaggttcccttttttatatctggagaaaaacaaatggctaaagaaaaagaagttacactggtaaacactggcgttgcactgattattattgatcgcgttgacgttatgccaggtaaagaaattaccgttgcggaaagcgtgttagagcggcagggtacaaaatcactggttgcacagggcaagctaaccgtaaaagataacagcgagctaaacgataagatcattgaagcgttcaacgaaaaacgcaagccaaacccaaccgaaggcaaaagcaaagcacagcttgaggacggcggcgagtatcaataaagaaagggcgctcatgtagcgccttttttattatggagaatacaccatgaatgatattgagatcctggagcaaatttataaaatcgcgccagcatttaagaaggttgatcctgaattaattcaggcatggattgaactggctaaagatttcgtttgcgaaaaacatttcaaagacaaatatttccgcgctgttgctctttacactttgcacctgatgactcttgacggagctatgaaacaggaaagtgagagcgtagaaagctactcgcatcgtatagcgtcattttccctgactggtgagttttcgcagacgttcagcaaagttagcgacgacacaagcggtaacacgttgcgccaaacgccgtggggtaaaatgtacgaagttctcaacaagaaaaaaggcggcggatttggccttacaactgcctttcatcggaggtgctcacgatgaattatgaacaaatcagatctatggcatcggctggaattaatttttttagcgatggaacgggtgaatttgattgcattactcaacctggaagcgttgaaattgttggcggtattgaggttgaaaaacctgaaataaaggtgaagattaaaggtcttgtgagagcaccacggacgcgagaagttgacggagagacaattagagttaccgacaagttaggtgtttttaatgctgatgtagagcttaaaaacggatatcagattgatatcgacggcgagcgttatgttatggttgaaacaagaccaatcagaccaacaagcattaccgttgcttaccgtccaataatgcggagggttgcggttcatggctgattattcgatccgagaatttcatggcaatgttgacaaatggatcgagcaagtagaaagcggattgaatgacgtgattcagatttttggtgaaaaggttcacggagcattagttgatattgcgccagtggacacaggacgattcaaagccaatatgcagatcacagcaaacaagccgcctctttacgcgctcaatcaatatgatcctgatggtgagaaaacaaaggcggaaggtcgcaggacgctttatgctttattgcatggcggcggcgctattaaatcaatatacttttccaatatgctgatttacgctaatgctcttgaatatggtcattcaaagcaagcgccagcgggggtatttggtatcgttgcgatccggttgcgctcttacatggcggaagcgataagggaggcgagaaagaaaaatgcactatgaattatctgttgcggcaagaatggcgctggctcaagaatacgaaagcgaatatatgatcgcttatgaaaacgttgaatttacgccaccaaaaggcggcggcatttggctgaaatatgactacaaagaagcagatacaataattcatgatctgaaaaggaaatgcattagttatattggaatggttcagatcggaattgaattcccgccaggctcaggaattgataaggctcgcaagctggcaaaaaatattgctgatttctttgaagatggtaaaatgctttcgaatggctatatttcagaaggtgcaaaagtgcatcaagttcagaaatcagaatcaggctggttttatccagtacgtttttatgttcgttatgatggttaataaagggggctattatgcacttacctaacggatcacagatttttattgagaatactcgcggctcagaaatcaaagcaacagcaatcagcaacgcaacagatccggtgtttaccgcgtctgatactggcggattgaaaaaaggtgattatgtgattgtcactgtatcaacgtggggtaaacttcttgatcgtgtactgcgagttaaagaggttagcgataacacaagtttcacgcttgaaggtatcgacaccactgatgaaaacgtttttccgtccggcgctactggtagcgttatgaaaattgaatcatggacagagatcccatgcgtacaggacttggggcaggacggcggcgagcagcagtattacaattaccagtgcttgagcgatgaccaggagcagcagttaccaacctataaaagcgcggtgtcactgacttacacctttgcgcatgagtacgataacccgatttatccgctcttgcgtaaggctgacgaatcaggcgacgttaaagcattgcgtatgtatgtaccaaaagcaaaagaaatgcgcctttgggctggtgtgctttctttcaacgagatcccacaaacggcggtaaacgaaatggaaaccgtttctttgtcggtatccctgaaaggtcgctttactttcttgccgtctactcaggcttaatttaaaggggcgcattgcgtcccttttttatttgatgtaatatcacctcaatatttaaccactcaggagaaatgacaatggctaaaatgaaattgactcttgccccgcttccagatttcaaacttccagttaaatttgtattgccggatggtaacgagcaaaaaattgtgtttaccgttaagcataaaaaggccagcgagattcaagagctttaccagaaagaaggtattaaagatcctgaatttatcatgaatgttgccgtgggttgggatctggaagaagaattcaacgaagaaaacgcgcaactgttagttgattattatcctggcgcggcgctggctcttatgggatcttatttaagtgcgctggcagggcaacgcgtaaaaaactaaaacgggcggtttatctgtattatcagaaaccgccgacagatgcagaattgcaagccgtaggattaacccgcgcagactatgaaggagaagatccgccagaggtcatatttgatgaaagtatgatgcaatcatgggatatattttgcgcgatgggtacacagtggcgtagtgctggcgctggtgcttacggttttgattataatgttttgcctatgttattcagaatctataaaatagacgatgaagaaatggccttaaatgacttgcgcatcatggaacaaaaagcactggaaatgatgcaagcaaataacaaataaagcgcctacgggcgctttttttatgagggtaaaatatatggctgaacaatttgcaggtttaacacttggcgttaacgtcgagcagttaaacaaggctgtaaagtcattgcaggactttaaaaaagcaaacgacgaagcaaagaaaggcgttgagagctttgttaatgaggaagaaattgcaaagcaaagagcaaagcaattagctgatgaattggcgaaacagagtcgtgaatttaaacgcattcaggacactgttgatccgacagcggcgaaaatgcgcaagctatctcaggccgcaagtgatcttgataagctatggcaaaagggcattgttccagatgaaacgtttttccagttggggagtatgctggaaactcagatcagtaagctggagcgtaataaaaaggcgttaacggaagaaggccgcgcagcacttgaggaatcaaaagccaaagagaaagcagcgttagccggaaaaactttccttgcggatctggaatcgcaagccgccgcgcttggcaaaacaaaatccgaattgctggaaatgaaagcggcgcaacttggcgtaagtgctcaggccgcgccattcattgcacagcttaaagcgcaagaaaaacagatgaagttaaccggaattagcgcggggcaatacaagcaagctatggcgcaacttccggctcagatcactgacgttgtaacgtcgctggcttccggtatgcctgtttggttggttgcaatccagcaaggtggacagattaaggattctttcggcggcgttggtaatacgtttaaagcgctgttgtcgtttctgaatccggtaaacgttggccttgccgccttagccgtttcaatgggtgctcttgttaaggctggttatgatgcgtataagtcgcaacgtgacttgcaagaggcgttggtgcttactggtggatatgctggcacaagcggcgcacagttcgataagctggcgcaggacattaataacagcactgatgcgacgataggaaacatccgatccatcgcaacagagcttgcaaaatccggcaagttcacgattgatcagattaaagcaatcacgaaaactaccgctcagtggtcaacggtaacgggtgaatcaagcgacaaaataaccgattactttaataagattgctggcgatccggttaaaggtcttgcagagctaaacgaacaattcaactttttaagcgaagggcagttaacttatattgcagacctggagaaaacgaaaggcaaaacggaagcggtaacggcggcgacaaagttatttgctgatgttatggatcagagattagctaaactggcagactccgcaacgccattagaaaagatgtggacgaacattaaaaaatgggcgtcagatgcatggggttgggttggagatcacacgctggcggcgcttaacctgattattgacgttgtagccggaaccgttgagcaggttcgttatttgcttaatcagggtgatattctcattggtgaatttattgtgtcggcaaccaaaacgatgcaaaaaatcccaggtcttgataatgttggcgattctgttatttcgcagcaacagaagattatcaataacgctaaggagaacaacaaggagctattaaaatcaattgccgaacgtaacgagcgagtcaaaaaaggcgagcagggttacattgacatgatgaaaaatcgtgcggcggttgaacagcaatatgcgacaaaaacaaaagagaatatcaggaaggaagccgaggaattagccaaacgcgataaaaagcaaaaggctgaaaaagttaaggtatcggctggcgataagctggaagaacaatatcagcgtgatatcctggcgttagaaactcaattaagagttttgaaagaacacaagacgatcacggataccataagccagcaacgtaaatctttatgggctgaacaagctaaaattcagattttgcaagaagcctcaacaaaacggcaacttactgatgaagaaaaaagcattttagccaacaaagataagatcctggcgatggctgaacaaaaagctattttaggcgatcagattgttgcacaaacgcgccttaatcagttgcaagatagctcaatcaagtttatccagcagcagaaagcagcaacagaagcactagccaaaaccagagggttaagcgagcgtgaagcagcaagagcggcggaacgtgcaaaggttgaatctgattatcttgctaagggaggacagaaagaagatccgcaacttaaagccatgatggatgccttagacaaccgatataaagaggaagatgctaaacgtgctgattggttggctggcgctaaaaatgcatttgcggagtacggcgaggaagcaatgaacatgtatgataatatcggcaacattgcgagccaggcgctaaacggcctatcgcaacaaatggctgattttttgactactggacaagcaaacttcaaagactttgcaaaatcgatcattagccttattgttcagatgatcacaaagatggttatcttcaatagcatttccggcatgatgggcggctcaacctggacgcttggaagccttttaagcggtgctggttttgctggcggcggttatactggcgacggtggaaaatatgatccggctggcgtggttcataagggtgagtttgttttcaccaaagaggcaacgcaaaggatcggagccaaaaacctgtatcgcatgatgcgaggttatgcaaacggcggtcaagtaggaagcgtgacaactggcggcgctggcatcaatcgcggtgcatcgcaatttgcatttggtgatatcaacgttaacattgacaacggtcaagatccaaaagggatggaaactggtattaagatgattttcactgaaatgattcaacggtcatgttcgcaaggtggcgaggtttataattttgttcatgggagggcttaatgttagatgaatttacctggtgtacgcagattcagagcggcggcggtgtaatgacaactaccaataacgacagggaagttgtttttggtaacggttatcgccaaaaggcatcttctggatttaacacggagcggagggatttttctattgtttacgctggcactgattataaaaaagtgaaagcgttcatgacggatcacagattaaagcctttcttgtggaggatgccggatggtgatttaggattgttcacagttaaagccggaacggtaggactaacgccaataagcccaaccgctcaggaagtcaaagcaacattcacggaagaatttacatccatgcgataaatcaaagccgccattgtgcggcttttttattgtgtctataatggtgctttattggagggcatagaatatggctgaaaaacagatcaaaaagacttttgaaaattgtctgcaatcgcttttccctggtgaaataattacgcttgttgaggttgacggcacaaaatttggtgcgcaggtttaccgctttcatgctgaaaatatcgcgtacacgccagaggaattaatgcaagcgcgtgaaactggaatattgccaccgaaagatattaaattccgtggcgaggtttacggggcgcggccttttgggattactggcattggctttacaagtaatggaaaggcagagaaaccacaattagcgctttcaaatttagatagccgcgtatctgcattaatcagatcatataatggcatgatgcaagccaaagtaacaatttgggttacatcggctgatttaattgatgaagaaggaaacgtggaggacggcgcatatagaaaactgatttattatattgagcgtccgaattttgttaatcagacagttgcacgttttgaattaacatcaccttatgatatggatggaattatgatcccgccacgactcacgcaaagcgtttgttattgggcgcaaaggggatggtatagaagcgggaaaggttgcggatataacggatcggcaatgttcgataaagacaataatccggtaacagatccgagcaaggattattgcgccggaacggttacagcttgtaaattgcgctttggcactcaaaatgaattagatttcggcggttgcgcggttgcatcactattgaggaaaaaccaatgattaacgcaaaaattaaacttgaaattatgcgccatgccaatgacgtttatcctaatgaatgttgcggcctggtaactcaaaaatcacgcgtacagaaatatcaccggatcgacaacgtaagcaaggagccggaaaaacattttgaaatggacgcggaacaatatgcagaagttgaggacggcggcgctgatatcatagcgatagttcatagccatacaggtgagggagcaaccacaatccctagcgcacacgatttgtgcatgtgtgatgaaactggtgtttcttgggttatcgtttcgataccagagggagacatgagaatcatagagccacaatcacggcctttgattggtcgcccctggtcgcttggcgcttatgattgttgggggcttattatggcctggcataaggagcaaggcgtgatcctgaatgatttccgcaagccgtatgaatggtggaagccggaacatggtgaaaacctttatcaagataattacttgaaagaaggttttgtagaaacaggtgaaccaccaaagccaggtgatatgatcataatgcagttgcaagcgccagtgtggaaccacgccggaatttatttaggcaacaatcaactattgcatcatgcattcggtaagttgtcgcgagttgatttatattctggatggtatcaggaacacacaacaatgatttgcagacataaggatctgaaagatgaataaggttatcaacgtaaaattatcaggctcattaggtcgccgctttggtgttttccataaatacgccgttgagagttggcctgaatgcgtaagggcattatcaagccaggttgaaggattcaaagaatttatgcaaagcgaaatcggatcaaagatgaaatttgcaattttcgttgatggtaaaaatgttggtctgaataatgatagcgcatggcgttgcgctcgcgaggttcgaattgttccggttccaacaggttcaaaatctggcggtttatttcaggttgttttgggtgctgttattatggtcactgcgtttttcactggtggtgcatcgctggcggctatgggtgctttttcatcggctgcatttatgatgggtggcgcaatggctcttggcggagtaatgcagatgattagcccacaacaaggaggaatgagaatggagagccaaagcgcagcaaataaaccatcatatgcgtttggtggtgctgttaatactacggcggcaggttatccggtatgtttgccatatggttatagaactgttggcggcgctgtattcagtgctggatcatacgcagaagataaactttaattatataaacccgccgttgcgcgggttttttgttacatgtacaatggcagcacgtttaaatagcacaaaaggttaaaagctatgatcaaaaatatgataactggcagtaagggcggttcttcaaagcctcatacacctgttgaaatggaagataacctaatttcaattaacaggatcagaattcttttagccgtttctgatggtgaagttgatccagacttttcattgaaagatttatattttgatgacgttccggttatgaatcaggatggttcactaaactttcagaatgttaaggctgaattcagaccagggacgcaaacgcaggattatattcaggggtttactgatacagccagcgaaattacagttgctcgcgatctgacagctacaacgccttatattatttctgttaccaataaaaacctttctgcgatccgtattaaaattttaatgcctcgcggggttactcaggaagataacggagatctaacaggtgttcgcgttgaatatgctgttgatatggctgttgatggtgctgaatataaagaggttttgcatgatgtaattgaaggtaaaacaatgagcggttacgacagaagccgccgaattgatttacctgctttcaatgagcgtgttttattgcgcgtccgtcgcctgacagatagcacgtcagcaagggtaacggatctgattaaaatgcaaagctatgcggaagttgtagacgctaaatttcgttaccccctgactggtttagtatatgttgagtttgacagcgaattatttcctaacgcgttgccgaatatcagcattaagaaaaaatggaaaatcattaatgttccgtcaaattacgatcctatttcccgcacttactcagggtcatgggatggaacctggaaaaaggaatggagtaataaccctgcttttgttctgtatgatttgattaccaatcagcgttacggactcgatcaaagagagttaggcatcgcgcttgataaatggagcatttacgaatgtgcgcagtattgcgatcagatggttccagacggtaaaggcggaacagagccgcgctatctttgcgatgttgtgatccagagccaggtagaagcatatcagcttgtgcgtgatatttgttcaatcttccgtggcatgagcttttggaatggtgagagcctttcaatcgtgatcgataagcctcgcgatgcatcatacatatttactaatgacaacgtggttaatggtgaatttacttacacgtttgccagcgaaaaaagcatgtacacgcaatgcaacgtgactttcgacgacgaacaaaacatgtatcaacaggatgttgagggagttttcgaaacggaggcggcgttacgctttggatataacagcacgtcaattactgcgattggatgcacacgacgcagcgaagccaaccgccgtggacgctggattttaaaaaccaacgtcaagagcacaacggtaaactttgctacaggcctggaaggtatgatcccaacagtaggcgatgtgattgttgtatcggataacttctggtcaagcgctctaacgctgaatctatcaggtcgcgtgatggaggttagcgggttgcaggtgttcacgccgtttaaggtagacgcaagagcaggtgatcgcattctggtaaataagccggatggtaagccagttggtcgaaccattgcgcgtgtgagtgatgacggtaaaacgctgacgctaaacacgacatttggttttgacgttcagccggataccatttttgcaattgagcgaaccgatattgcacagcaaagatatgttgtaaccggaatcactaagggtgacggtgatgaagaatttacctacaacataacggctgttgaatacgatccgaacaaatacgatgaaattgattacggcgtaaacattgatgaccgtccgacttcaatcgtgcagcctgacattttgccagcaccgcaaaacgtcaagatcgaatcttattcgcgagttgtccagggcgcgagcgtggaaacaatgcatgtttcatgggataaggttgaatatgcaagcctttatgaaatgcagtggagaaaagacaatggtaactggaacaacacgccgcgcacagccaacaaggaaacggaagttgaaggtatttacgccggaaactatcacgtaagggttagatctgttgcagcgaatggttcagcgtctggatggtcagccattgtaagcgccggattaactggcaaggttggcgaaccggaaaggccaattaaccttactgcgtctgacaatgaagttttcggaattcgcgtaaaatggggtatgccagaaggaagcggagacacggcatatattgagttgcatcaagcgccaaacggttctgatggtcatcctattgttgatgaagcaacgctcttaacgctgataccgttcccgcaatatgagtattggcattcaatactgccagcggggcatgttgtctggtacaaggcaagggcagttgacaaaatcggtaatgtttctgattggactgattttgtgcgcggcatggcttccgacgatacgagcattatcacagatcatattaaggtcgatattgaaaattctgatggctataagtggttacaggaaaacgcaatcaaagccaatgataagatccacagcacagcggaatcagtgattgaaaacgcattagcgaatgataaagatgtgcgacgtatgcgagttgaaaacggcaagcgtaaagcggagttcttgcaatcgctgaaactcattgcagacgaaacagaagcaagggtaacgcaggtcactcaaatgagtgcgcaatttgacgagaaattaacggctcaaaatagcgaattgagagaggtaattgctaacagcactgaaaccattagccaaaggattgatcagcttacagctacgtttgagagcgaaattgacggtgttaagcaggatatcaaagcacagataactgatgttaaccaggcaatcaccaatgaagcggaagcgcgagcgtcagcggatagggcgttatcaactcaaattggtgatacccaatcagcggttaaccagaaacttgattcatgggttaacggtacaagcgttggcgcgatgtacggcgttaagttggggatcaggtacaacgggcaggaatatagcgcaggtatggcgctttctcttgtcgctgatggtagcggagttaaatcacaattcctgtttgatgctggaagatttgcgatcatcaacaatgctcaaagcggagcctttacgttgccgtttgttgttgaaaataatcaggtgattatcaatagcgcagtgattaaagatggatcaataacaaacgcaaaaatcgccaactttatcaactctaacaactggcagagcggggttgcaggatgggctattaacaaagagggttacgctgaatttaaccaggtaactgtaaggggtacggtgtacgcaaacgccggatcattcacaggtaacgtttatgctactgatggatggtttaggggtactgtttacgctgaaaaaattgagggtgacgttgcaaaggctgttgttcttgggtttaacggatcggttcacattccggctgttaactacaacagacaccttgttattccttatgttggtttgcatggttggacgtattcaggcggatacgggcagggcggcggtactgtttgggtagattcatcatatggcggtcgaattgctaacgttagttcaactgcgatgcatggcggcggtagtgcttacattatgttgccagcaggtaatgcaaccacgctttcttatggtggaagtcttaaccacgctaacgcggttccagttttaacggttctgttattcaaagtataaaaaggaggtaaatcaatgccctggcttaacggtcggggcattttgttttcttttgttccatgatgttcacaactgaatccggcgtaaaattgaaaacgtcttgataacaaaaagaacaaataaaatatatatataatacatacacttagagtttaattattattatatatatctatttgtattcttattgtttctattgttctctatggtttgtagtggttgtgtgttttgtattggttgatggttatatatcacatggtatgtatttatatatggtatgctgcgtaaatgcgtgaacaatggaacacaaaaacaacaatcagtaaaatcataaacttacgatgtgattttgttgtcagtgctaattgtaaacgtatgcaaaacaaaaagaacacttaaagggtattgactacatgcaacaaatacctataatgcagacataccaacgagaggatgaccaaatgagcgattttaaagtttacacctttgaccagttaaccaatgatgaatatcacgatcctaaaggttgggcggctgaatacgttagcggctcaagcctggcggaaatctttgcaacttgtccggctgcatggaaatacagaccacgcgaggaaagcaaagcgcttgttttcggtacgcagtcacataccaactttcaaagcagcgagttgttcgcaaaagagtaccgccgagcgccagcgccggaagattacgaaaacttgatcacaagccaaacggcattagccagcaagttaaaatcttttggcctgactggtacaagcgggaaaacttatccagacctgataaaaatgatggttgattgtggtgaggatcttaatgttcagtggctgattgaaatgatcgcagagagccaggcgcgagcggataacgttgaattggttccggctaaagattacgacgcttgcgttaccatgcgccgtgtactggaaagcataccggaacacaacgcttgcatgaatagccaaacagcacaacgcgagctatcaattttcggcactatcaaaggcgtgaaagtcaaagtaaggtttgatcatgttgatatttgcaaagatgtgcttgcaacggtgatttccggttatgatggtgaaggtaatccggtatatgaaacaacgcgatttgaggaagcgattgtgatcacggattataaaacaacgtcaagcgctaacccgtcagagtttgcaaggctggcattcaatcacggttactatttgaaaatggcattgcagcatgacctattcaagaaagcgtatccagaagaaacgcgccccgtagtcattagattgctggcgcaggaaaagaaagagccttatttaccgatcgcataccgcatgacggatgaacaattgaagatcggacggcttcaatacatgagcgtgataaaccagtttgcagaatgccaggtaaatgacgtttggccttcatacgcaaacggagagccggaaattgacctgcaaacgcctgattgggttcgtcgtcaatacaaaggttttttataaatagcactttttgctaaaactgccattcgatgcgtggtatactacacgcatcaaccaacgagagagaggaaataaaaatggaacagcaattcaatgagcaagacattcaagatgtgcgcgatcacgtaggcggatttgtaaacgcattaggaattatgcaacgccagttcttgcgcgtaattgatccaagcgaatcaccggaaactcttgaatatgttcgcaaggttgcatattcgattgataacgttgtgttaacagttcttcaaatggaaaataacgaagaacaccgcaagatgattaatcaggcaagtgaaatcatgatccagaatttgattgattatcacaacgagaacgaagaaaagcattaattaacacggcggcgcaagccgccattattaaaccggagtcaatgaaatgaagttttccgaacaaaacgcaaacgtaattaaggcattgtttgaggcgcgtcaattattcaccaaagtaaaaaaggataagcaaaacacgcacctgaaaaacaaatatgcgactcttgacagcgttcttgatgctattatgcctggccttactgataagggattattccttacccaagatcagaaagttggcgaagatttaaaatcaatgacggttttaactcgcttcattcacgttgaatcaaatgaatgggttgaatatggcttcacgttgccaatgcagaaactagatccgcaaggtggcggctcaacaaacagctatgcgcgacgttatgcactttgtaccgctcttggcctggcaacggctgacgatgacgcaaatcttgccaccaaaaacgcgcaggattggaaaaaagatcttgatgcttgcgataaccttacagacttgcaagaaacgtttaaaacggcttacaagcaatctgatgcggctaaccggagaatcattaaagagcattacgacaagctaaaagcgaaaatggagatcggaaaggctcgcggttttaatccggctgaaccagcggtaaatgttgcgaagaatgaaaaagttgaaaaccaaccgcaacaggaagtaaaatcacaatctatcactgactttgaataatttatcgcggggcgatatcgccccgataaggaaataaaatgcacgttatcaccggagaaattcgcaaagagccttttgttaagcaagggcaaaacggcacactgtatattgttgagctttcagaatcatataaggatcgagaaggtaacagacaatacaccaattacaaattcttctttaatgcaaaaagcgagggaatgaataattggtatcgtgaggcattccagcagggtaaaattatttctgtatcttgcgatcaattgcgcattgatagcagtgaatataacgggcagatttataacactctaacaagtgccggatttgctaacctgcaatttagccagcgcgtagggcagcaacaagcgcctcaacaatcgcaaggcggatggggtcaacctcaacagccacaacagcagcagccacgacagcaagcgcctcaaaataatgagccgcctatggattttgatgacgatattccattctaaaaataaaggggcattacgcccctttttcactttctttgatacccaaaacagcaattaccttaactgcgatccggcttgcaagttcaagctgttgtgcgttaagcgttccggtattcatgatcgataacagaccagacatagatccaagagctttaaaatcagtaaaggctgatttcataatatccatttgttgatgaccatcattcacaatcgcttgtgcgcgttctgataggtctgcaactaccttttccacaccatcacttttcgtcattttctgctaccttttccgcctgagttgcttgttgtggcatgttaggccacgggctaatcgcttcacgttctgcaacaggcttttccggttcaatccctaaaaacttgccaacaaattcaccaacgaaattagagctatcgcttacataaacgtgcatcctatccagatccgattgactcactgagtgaggcgcgatcaagattcgttgagagtgaaaaccgatatacatcacattaattggacgttcaattgtcccaggagtaggcgcaattacgcccataatccaacagtagtgtatattgccgtctaatggttcacatccggcattaagccagtttaccttttgatgcggaaacaccattgatgaatcagttgcatcacggcgtaaagaccaatcaacgccaccaattgaaattgtgtgtcgtggcttcattccgttaaattgcgttaccccgatcccgtttgtgtttgtcgtttgcatatcggctcacctcttacattgatttcatttttttaagcgcacagatacaagcagcagccagatccgttaattctttttcgatgccatgtttagagctatcagccttttcagccattaactctttgtactccatttcaacaattgacatcatgccgcctggcttgtcgtggtacgcttcccaggtgtgcggagggtttgaaatgcgttcatgaacatctgccatagctgaaccatgttcatgagttctagaatgataaccatcatgatctttttcctcatgcgagccaataacaatgcgaggtaaatgaatgcgtggcattcgatatccagcgatcttaatatgtgacataaaagcctccatagagaaaagcgccccgaaaggcgcttatttattagcagccacagcagccagttggcggcgttggcggggtaggcagtttgaagttaaccaactgctcaacctggttgattttgcaattcagcgcggcggtttgattcgcctgagacagtgcaaaacgagcctcttgcagttgcgctttcatatcgcaaatgagaacagcctgagaatcagcgaactgttgacgcagcagatcacgcgttgcattaccctggcgctcaatgttcaggtttgtctcgcagcaacatctttcagcggcaagttgcgcctcaaaagcggtacgctgttgatcaaacgactgagaagcaatagcgccagcaatggtgttagtgctttgagtgatcacagtgttcagacctgcaaatccctggacggatgccaaaaggttctgcgtgttctgattggtaatgccgttaaaagttgaagcggcggagcgttcaacagttaagtttgtcgcgttctggccttgcagggtttgcagtccaaggttgttaacgccagtttgaatgttattgataccatccagcacagcgcttgcgccaacagcaacggcagcaccatcaccaacaccaccattaaagccacgaccaccaaagccattgccgaaccatgaaccaatcaggccaccgacagcgccgccaagaccagccgcaccagcttcaccaccaaaaccgccgcccgtaggcaatagagtcatatctgacataatgaattcctctttgacattaaaaaaaattaaaaaatttagtaacactttcgttacgacaaagggtatgcgccttattcacggcaatagatatgggttcattgtaaagaattggtaaataaaaagggaggcgttgcgcctccctataaaataaaacttatttttcatcaatatttaaattctgctcataactttttaatgattcttcgatcatcttgttatgttcactgtcagatggcatttcaacacgtaaatcaagccatcttccattaggtatgtcaattggatccccatcagaaaatccttcaatctcatttctagcgaatttaggcacattatcatgtgttctatgataagtttttacaattatccctccatcctcttttatttcataatcaacccaaagcaaaggtaatccattgttattgttaggaataacaaatccaccatcaacaccaccccattctggacttgaattaaaacctaaaacgcctgatatctcataaacaccacgacaaactctaacagcctcagcaccccttgattgatcgtttgtttcaattgttccatcacccattagttttatgataggtgatgcaaccttccaaaatccattagtgtcaacagtagcattaagtccttgtacaacaaaattaaaccacttgctaagaagtgctggatttgcgtttctcctacgcattttaggtgctgcattaactagaattggaaatgcaacctgcgtccaatttccaacagatccgttattttcagatagcgataaaacatatgagtaattgaatggatcgccgctacctccagcatgactaccatcaccataataaattataccagattttagatacggtgaatccatttgcaaaccatcaacaattgcactcatagcatttatgttgtcacacgcacctcttgcattgtcagaacctgtaccaccctgtgcaacaccaagcgttttccagccacctacagcatcattccatacaccccaatcattattattgtttattgttattctgtatggagaattaccaggcccatacatcatagtttcgtttgctgtttgcttgaaacgttccaaaccaaagtttacctttgctccttctacgtttccagcaccagtaccaccttgcgccacaccaagcggtatatatgattgagattcttcgttaaatccaccccaaacgccagtatcactaacaagaaccgatcttgttttattgcctgacaccattgatgtaatagaaccaaattgattaaacctatcaatttgaaaattaatttttgcctgtgttacatttccagcaccagtaccgccagctccaacagaaagtgcagatgtcgcgctatcactatttcttgcaactcgccattcgccattgttagctattcgtaattcataagtccacgcagggttacggaaagagttataagcgccagtagcttgatcttttacagaattaacagcatcaactttcaacgcctcccttgcaaggtctttgtttgcaagatcattaaggttttgatccttatgcagcagattttcaggctgtacctgttgcgcccattgacgcgcttcatcacgcgcagtctcagcgccagattgtgcgttatttgcagcagtctcagcacgttcagcgccttcttgtgcagcccatgcaagatcttttgcgtcaatagcctcgttctttattgctgttatttcagtaacggcagaatctttaatctgttgcgtttggttttttattgcgtccgtgtcggccttaatctgattggtttcagaaacggcagcatttttaattacggttatttctgtaacagcagaatctttaattgcttgcgtgtcgttttttaattgttcagttgcagcgcgatcagcagcggtgtttctttcagcatccttgatctgattaaccaaatcctgcaattgtttcaggtcaaagtttttgagaaattcaatcgcttcctcaataacagtttctttactttggtaatagcgcagagtttccgcaacatcttgcgccattccatccaccgttaaagagtcattcaacaggatcacataatttccgttttctactggctcgccgtcagtctggatcgctttcatttccgtgtcgcttacaatatcgctaattacagccaatttaagtggcgatgtgagaaatacgattgttgcgccagtgcggataagagaaagaggctcacgccattttgtgccagtaccagtaataacgccttgtgcgtccattgcagccgtaccagtgcgatataaagccataattacacctcattttattcatttaaacagtgtagtgattatgcgatattgctatgttacatgcaataaaaaaccccgccttagcggggttcgtcaacgacaacgagaagtcaatgagcagtagccatcgaaaagtgttagtagcatgatgaatattataataagtcacttgccttgtaaagcctccgctctattccatttttgaatttaccgttaactggtaacattacgtctaattcgcgatcatcagcagcaatcaaagtatcgcgatcattagcatggcacacaacgcgcattttgcgatctcctttgtatctgtatgcaacaatttcaagatttgaaggcgagaaacacgcccatacttcttgccccgtcgccaggtgtatatgttgcgcatcaatccaatcaacacaaaggtaaattgttttgtcagtcttgccagttaccgcaacgcttccacgcgtgtaatctttagcgtaaaaactgcttttaccttcctcatttacgaaaagaatattgcacatatcatcatccaattcatcactatgcaccaggtaacaaggaatagcgtgtatatgctcgcaatacccattatcatgagttttcacgccaacttgatacgattcgccgtctaatggataaaacgcctcaaaaacgcttaggtttgttatctccatgcgctcagttttatccatcacctcaacacatttttcatgaggtgcttgcgatccgaaattgtaaccggaatcacgccttgccgctttgttagccttgttgacaacctcaaccggaaccaggtttaaccattcgcctagcgtgtcgattgcttcactgtaattttcaccacgtatgcgcatgaacaatccgatccctgaatcattaccgcattggttgcaaatgttgccgccgtcgcctttctcgttgattttgtcagtccaacgaaagcgatctttaccgccgcaataagggcatggttggtgttttccgttaaaggttaattgaggaatatcgcaaagtttcatcaatgccgcttgccacataccgcgcatctttgggatcacttcttctttttgataatgcatttctcaatctccaaataaaaaacctggcacgatgataaaccatgccaggccgtaatttttagcaatcagtgctatccatagtaatgattaccttcttcttctgattggcttcctttgctttgagccgttcttctttcgccttgtcgcgttcagccttgcgccgcgctttctccttctcgcgttcttctggagtcatagaccgtaacggatcgtaaggccggacaaatccccttgcttccgcttcctctttggttacaaatcgcatcagctttctgttgctgcaacgattctctaatggcttgccgttattatcaaatttgatatctggacggcaatatgaagcgcggaaaccgttaaaacccttgcggcggtaatctttctggatcagttcagcgcctttagttgaaatcatgccgcgctctttccatccccacactgttttaaggttaacgccaatttcacgcgccagggcagcgcaaccgccgtaagcgttgacaacttcatcaagccgcgcttttaatccggctcttacttcttcctttaatacaagataaccgctaacaggggcgcgacgtttgcgatctttaccacgtcttacgccgttgttaccattgattgtgcgtttgtcgatttcgccagttgaggcggcaattttgatttcgctcatattttcacctatagcactttttgctaaaaaacttgattaagtttgtgtatgatatgccgcaaatggcatttagtcaaaaggataaaaaatgattcctaacattgaaaagcagattgcagatcttggagaggaaaggattaaggcaatccagaagcgtttcacagttggtgagattgtcccgtatgaataccaatgcgttgcttacgctgaaattgcaaaacgattgagcaagtatgttgatccgtttttcgttaaagcggcggtatcggcaggtaaaacaatcatctttgctatggttgcggctcagtgtcggaaaatgggattgaaagcgttatttcttgcgcgtcaatctgaaattgtggatcaggattcaaaagagattagcgcctttggtgttccgaattccgtttattgcgctggcctgaataccaaaagcgcttatttcccgattgttgtcggatcggaaggtactgtaattggaggattgaataaagcgttaggtgattacgtcccacaggttttaggcatagatgaatgtcatcaagtcaactggaatgatattgttgaagccgaggaaaacggcgaaagcatagagcaaatgatgacaccgaaaggtgaaccagttgaagggcatccgttgctgattggttcaggccgcgcacaatacaccattatcattgcagaaatgcgccgccgttgtcttgagacttacgggcatcaattgcgcatatttgggatgacaggatcggaatatcgcggagttgagccgatacttgttgaagataaaagaattcctggcttttggcgcgagcagatcacaaacattgatactgattacctggtaaaatttggttcagttgttcccacttattttggcaatgttggtgatttgggttatgacttatcagaatttacgccagtgcatgaatttggcgttgctgattatgaccagaagcaattaaaggccatgagtgacaagattcaccaatcaggaaccatgacgcagaaaataatgcaaatggttcatgaggtgatgaaaaaccgcttgtgtgctcttgttacttgcgctgacgaaaggcactgcaaagaggcggcggcggcgcttccgcctggcacgaagtatcacattattaccggaaaaactggagagaaacagcggcgcttatggcttgaggatgcctatgaaggtcgcgtgaaatatatcttccaggttcaggcgctaacaactggcgtgaacgttccgccgtgggatacttctgttattttgcgtaagatcggaagcctgacgctattaactcagcttttggggcgcggtatgcgtcaattaaaaacttaccataaagaagtattggggatgcacaaaacggatcaccttgttttggatttttccggcacgatgtttgaaatgggtaatttgtatttcgatccgatgttagagcaagcgcaattccagttgcgtaaatcacaggacaaagatcctaaatattgcccgatttgcggcacagaaaattcattctatgctcgccgttgcattaatgaggatgaaaacggaaatcgttgtgaacatttctggacgttccaggaatgtgaggatcagattgatgaccgcacaaagaaagttattgtaaaaggttgcggcgcaaaaaatgacgttgcggcgcgtgtatgccgttgctgtaatgttcagttacgcgatccaaatgataacttatccggcaagatgtaccgtaaaaacgattggtgcgacgtgttggattttaatgtgacgctgacaaaaaaccaggctggcgtaatattctgctacacgttgcgtgatagtggcggcgtagaattcagggcatacgaaaaatatttccctgaatcagaacacaagatatgtaaaacgctttggacaaaggcggttagaacacatgttatagatcgccaagccgcaaactttatgatcacttgccgcaatgcaattaagatcatgagttatgcggggcaaatattgccgccaaaacgtgttacccatcgcaagaacacaaagaaagaagatttgatcgcacagaaggagttttaaaaatggttactgataaaggcgattatctggagtattacgacaaatcagatccagatgacaggaaagaggaggcgcaccaggttgattcttactcatggttaatatatgagtttcccatgtggcttgtttggcacactaaaaacgaaggcaagaaaacaattggcacagcagtaaaggatcagcaagcaggtgtgaaaaagggagtaagcgacattctaatcctgactggttttattggctgcaaatatgcattcattgccattgagcttaaaagagccaacaagaaaggcacgaaagtaagccaagagcaaaaggaattcttacgtcgagttagggaatgtggagggtttgccgccgtatgctatggtgtagagcaagtaaaattggctatcgcagacgctacaaaatagcactttttgttaaaacaggcgcaacggaatgcgccataatgcacccatcagaacgaaacggagaattaatcatgaaaaagatgttatcagtagttgctttatcacttgcccttgtcggctgcatggataaacaaaaaacatatatttgcggaaatgaagcgtttgaggttacaagcgactatatgaaagttgtgcaaggcaaaagcacaggcgttgtaatggataatatcagcaaaaatcaatataagctgtttactccaattggaacggcttattacactgttaacgatagcaccattgatattaaagttggcgtttatcagcacactctaacttgcgaggtaaaataaaatggctaaggattttcaggacaaagcaaccattgatacttttgagccggaaatggattttcatgaattgcaacacggcagatattcagatattgctttacgttcaggcggtcattatcagccagttagagaaaacccattatttaaggttagcggcaaccgttatgcaggaagtaaaacgccggacgttgtgcgcgatttgtgggaaacgccagatgaaattgttgattatcttgctgaacgttacggcaaatatgatcttgatgcagccgcgacggaagataataaaaaatgcgataagttttacagcaaggaaacaaactgcttaaaacgttggtggggtagcaataagcatgtttggctcaatccgccatacagcaatccagatccgtttgtgttaaaagcaattgagcaaatggaacacggaaaccagattgatattttacttccggctgacaactcaacagcatggtttagcgatgcacaaaaacacgcggcggaaattatctggatcactggcgaaacatgggaagaagaaggaaaacaatattcccgcactggtcgagtgtcgtttatttctggcatgacaggtgagccagttagcggaaacaataaaggaagcgtgatttttatcatgcgtcaactgaaagaaggtgagcaacaacaaactcattatgtgccaattagtgaaatttgcccttctgttaagaacaaacgcacaaaagcgcggagtatttaaaatgagcattcgattagctgataacatgaagtgggatctgtttgttggtatgacaattcgcttacttgataagcatggatacaacgccgataaaatggagttgataaactgcctggatatgacatttgacgaaataaaagttgccccgatctatatgtgggaaagaaaaattgaacacgatttaattcaatacaaacgcagagagggcgctcgattttttaatgtgaaatagcactttttgttaaaacccgcttcggcgggttttgtattatgcgatcaacaggaaacaagaggattgcaaaaatgaaaatcgtaaacgttgaatacttcaaagatgcacaatggttggctaaagaatctttgttcatgattgagcgtaaaaacccaaaagttattgcacaaatactttggaatgatgcaatggagttactaaaaaaggcttacggttatgaaagtgacaaaaggtaaaaaggaaacatgggagttagcgaaaaaaggcggtcttgatgaagggatcgccaaaatagcacaatatttcgatatcaaagacgtgtgcgttattgtcggtgatgaaatggcatatgttaatgaaaagccgcgacgtgttcatcgcgttcaggcaattccgacaacaatcgactataaagaagtgatcaacaaaaccaaagagcaaaggaaatattacaagtgaaaacaattaaattcatgtttgctgttgtcttgccgccaatgctggcatggtttatgatgtacgccgttgttatgaaataagaggatttgtgaaatgagtgcggcaaaaattacggacgaacaacttattgcagaaagggaagcaggtttaaaactgcgtgagattgcggaaaaatacggaatgagcttgcgccaggttgagcaccgtcacagcaaattagctaaacgtggcgaaatatcaacaatcggatcgcctggctttgctgtaattggtgaatcacagcttaaacgtgcaaatggtgaggttgtaatgacctggactaaaacgcacaaagacaaagcacaattagaggcaatcatgcaagcggctatggctgcattttcggaagaagtgccgcgcatcaagccgcagcctgaaaaaattactgattactcgcaaacactggcgttatatccgatctttgatatccatattggcgcaatggctcataagcatgaaagcggtgagaattacgacacggcaacggctgaaaacgtcatgaatgagtttttcgattatgcagttgataaagcgccgaatagcgaaaaggctgtattgctgattggtggtgatatgattcacagcgacggattagaagccgtcacacctgcaagcggtcacgtattggatcaagattcacgttatgcaaaactggtttatgttgccattcgtgccacgcgtcgagcagtaagccggatgcttgaaaagcataaagaagttgagatccagatcattgagggtaatcacgaccaatcaggaatgatctggctacgtgctgcaatggcggcggcgtatgaagatgaaccacgcgttaatgttgatgtttccccgcgcgtagtgcatcacacgcaatatggaaaaacattcctggcttatcatcacggtcacactgtacgcaagccagaaacgcttttaatgatgtgtgcagcggattggcgggaagatttcggaaactccaattcaatgtatgcgcatgtagggcattggcatcatcagaccgtcacagaaacgagcctgggcattgttgaggttcatagcactatggcggctaaagatgcttatgcggcgcgtggcggttggcgttcacgccgtcgagcggcggtaattatctatgataaggaatatggcgagatagggcgcttcatgttctaccctgaaatgattgcataattaaattattacctaaaaaggctggctaaatgctggcctttttttgtttttgtcgttaacataatcagactatttttgatattgactttttaatttttaagggatctagttatgcgtgaatttattaactttgcaacttattcaagtggtggtgcttcattctcaggcgctgcaactggtcaattactgttggcggttctaacattcatattttttgttattttcggcgctgttggcttgtggctaaggtggcgtgacagcaaggcgatccgagaagcgttagagtgtggagatcttaaaaccgctcttaaaattaaccaaaaatagggagtgaatttaatggctattaaaaagacaatagccgctaccataacgggagcggctttaatgatggcatcgcccttgattgaagaaattgagggcgtgaggtataagccttacaaggatattgcaggaatctggactgtatgccacggcatcaccggaaaggatgtgatattaggaaaggaatataccaggcgagaatgtgacgcgctacttgcaaagcatatgaaagttgcggctgacgctgttgataaagcggttaaggttgatattcctatttctatgcgagcggctttgtactcattcacattcaacgcaggaacgggggcattcaggaaatcaaccatgttgaagaagatcaacaatggtgatctgtatggtggttgtggcgagctttggaactggacatattaccggaacccgaaaacgggcaaaaaggaaaaatcaaagggattgaagaatcgccgcgctgttgaatataaatattgtgtaatggatctgaataaatgaaaagaaaattactggctattatgatgtgcattgctctttctggttgttcggcttcaagcgtgttacctggtctgattggtagcaagcctgatataacggcacaggctggatcggagaatgtgaaacagactgttggtgtaaccgcaaagcaggatacatcaagcaagcaggaaaccacattcaaagaatcggcagttggtcaagttgacacatccaataaaaagcaggtgagcacctcaagcattaaggctgataacattacggcggaaagaatagaaattcgcaacaatgacggtcttaacgttccggcaatcgcaatgggtatattgatgtttattgctggtatgctggcaggttgtacactaaaacgtaataaggcggcataagccgcctttatttttaaaaaatgttgttcattaagtcttgaaaactatttccgctataatccctttcctcaataatgtgagcgcctttcttttttatatccaacagcatcttatcatattcggaacggttaatgccaaaagtgctaccatcatcaacatatgcggttcctttgttattgaaaacgtcaaatgtaacattaacaacgcgagtggttttgatggaatattttaatgacattttgaatttccttttgttcgtttcgatgaaacaactatacccgatacaagatcgggcgttttaacaaaaagtgctattcgaattttcttacgtgaattagcaattcaccatcatgatttgtcagttgatgctcttgatccccaaacgctcgcatatcgctgtaaatcaacagcattaacgcatagacaaaatcatcatgcggaatttgcattgctgaactttctttcaaaataagatctatcaaatgctggcttgctgaataattcttcatcgctcattttctcaatgttcatcatttcgtaaatgtacacatttttatcaccatcaaggctatataactcaaaattaaacggccttttcatcttgtttttgcaccagtaaacacagtgaactccgtcaatatatccatatcccatcattcgcgctataaactctttgcttgtgcttgatgcgaaatagcggggcgttacccccgcattttttgcaagcctttcacattcacggtggcggaatataaatcttgctaactcctggcggttgaatttctcgcgagactcgcaaaagcgatacagatctaataagaacatgattaccccattaacgccggattgacataaacaaatttaccaacgctgcaaacataattcttttcttcaagcattggcaataattgttcctcaattcgtttcatcacgcctgattgacctgtaaaaggcttaaccttacgcgcactttcatagatcgctcttgcattggtaacgcctttattttgacgtgagatcttaatgatgatttcaattaacttgctcatttcggcatcatcgccagcgtgaccggaagcattcgcggcgttaatatatgttttgcttaattcgctgaacatcaccaaagcctcttgcattgtctcaacttcaatttctcgcgacttttgaggcgtaccatttggattaaaccaattgcggatcgtgtgcaatactgacgcaatgcggatcacttgtttatccattttacccaaagcaccacgcagcattgtatgtgaatattttccgccgtctgctaaatctggctctaattgctggcgagcaatattaagaacacgcatcgcagaacgtgacggcttcaatacaacattttgttcagtcatgatattatgaatcaactggaaatattgcgcctttaattctttatcaactggcgtgtaatcaagatcaccattttcatcaataaatacacgctcgcctaacattgttttttcacgcacaagcaagaaacgttcactaacaccgataccccgcgcccctgcatccatgatcccttttatagtttcatcctgggcaattacgctcatgcagcccaacgcaacaaaactcatattgttttcagagtttgcgcgagcgattgaaacgtgtccggcgtcccatgcttttagaaccaattcgctgttagtcttgcgatcactattagcgtaagtcaatccgagtaagctgttgatgcttgttgcttcatcggaaatgacagcaaagttaccctggcgattgttgatctttgctaacccttccggcgtggtgtctgatacaggaaaaacgatatcgcacattttttctaacttttcctctaattcctccttttcttcaaacaacgccgctaactcagtgccggaacgttcagctttcaattccttttcaacgccttttaatttagctgcgatcttctttctctctttcttgcgttgttcgttgattctctcgacttcacagaccattggagcaatagccaagctgttgattgctgatttgcctgttgatggcggctgactggttacaacataaagcgcagttggttgttcagtgccgtgatactcaacctggaagcgtccaaccatagcggcggaaacgcaaccaatgaaatgcatataggctgatgattcaggaaattgaacagattgcgcaatgctacgtgacagtttacccacaacatcaacatcattacccaaagagatcacaggataacggtcattagcgccgttgatatccttcacaggttcccaaaagctagacgattggcggtagccgttcgactgaatagcaacgcgaagcggcgatgttccctgcgattctgcaatggctataacttgttgaggtgtcaatttgttggtgtcaaagtcaaacatctaaaaatctccttcttgttgccgctaattataacggcaacattctacagccttttagcaattcgtgctatttgatcggctcaaccctggctaataactttccgtctataatgctatgacattcaagagttttaggatcgaatgcaagcgaacgatggaacgtgtgaccaatcagcaaatatgaatgcttatccctgatgcgatagaccgtttcaccttcctttaatgaggatattgcactgaaaacaaccttgtaacggtatttttgcatggcctcagaacggtgaggaatgtaatctttttccggtaaactcatgagtatttggcctcaaacagataagcaccgccagcggctgaaaatccaacttcttcccgatacagcgtgaaacgatcgccgttttcgtcgaacacgtatccagcaacagcacctaacacgcggccttgttcgatctggtaacgcttatcggctttgaaagttttcttgatctgcatggagtggtttacccatgtgcatttaatggttttggtcttaacgtggcgataacctggctcataagcatcacaccattttgaacctttcgccaactcgcgaacctggaacacctccatttcaacaacttcaccgctatcattaacaggaaccattaccgctttaaagagagcattcattagtttgttgtcagaattgcggataactttcatcgtgttttctcctgttgttttcgttgaaagcattatgccggatacgtgatccggcgttttaacaaaaagtgctatttctgatttgccaggcatacagcaagcgcaaatccgcgcggagtgagtgagcggatcagctttgtgcgcttagacttgccgccgagcttttgccagcctggattgttgttccctgttggcaatactgcgtttgtatccggcatcaaaaaaccgttacctgtccaaaggcaagtttttttggtgtacgcatcgcgagcgggaatgatttcaggaaagtgagggtgtttatcatcttccggcaacagaccgccaaacgcgcaaggatgaaatataaaatcaggttgccgccacatcgacgacaggacgctaacaggattctcgatcatgtatggcacgttgaaccaatccgcaatttcagccgcaatcttgcatgttgaaacggccttatcttggaatagcggatcacgttcacgctttgccgcaaagtgacgcgatccgcttacagctaaatcagtgcatggaggaaatgaaatcacaaaatcaggctttccgtaaacctcattgcgagccttaaaactgaaatctgaatcaatccaaatgtttacatactcaatgttgggatgacttacgcgtactgaatggtaatcaccatgatcagcatcatcatagttaaagcagattactttatgaccacgttcagcccacggcaacgcatcataaccggagccgctaaaaagagaaaaaactaacatgcttaaatcctttgtgtatggccttgtaaggcgttttgtttgtaggtgttgcaatcgtattagtaacggcctgaaatcgcgaaacgacgcgaaacaggccgctaattttagaatgggaaacagcgcttgcaagatgggtcgaagttacaaccgcaatcattgattattgcgttaggatcagcaagcatcacgctactttcttgttccatatccatagcgcctaatgcgtcatcaagcgttatttcaagataggcaactttgagcgcccactctttattaagtccagccgcctccgcttcatgtaggcgagcaaagaaagcatcttgaaccatatcaaccccacttctcaataaacatgctcatttcgtcaatcatcgcgtaagcatcgctttcagtcatgcggcggattctaccgcctggatgttcgccaacagcgtaaccagtttttacgcgggtaatcgttaactgatggaatccagaagcgcgatccattttcaaaactacttttccgtgagttttgacaatgttcatgatggattcagttttcagtttcatttttgattctcaattttgtttgtgtgaaagtatgataccaatcagagcgatatcgtttttaacaattcgtgctatttacggcgataaacggatatcctgataaatctgcatttctataacgtgcagacctaacccaacagccgcctttgctcataattaaagcgtttttatgacgcaggttcacgcggtaaattcttccggtttttatgtttttaaaataggtcataatttaacctcatgatattctataaattcttgcacatatgcgcattcgcgcggcgactcttggccttttagcacttcacagccaccattaaacagatagcaatgctggcaaaggttgccgcctacgctttcctcaacaatctgaattccggtacatgcgccgccaccaaaccgatgaggaaaatcgtatgcctggcagcggcacgttgcttgatgccgctttctggtcattaaatgttctcgctgataaatttgattacttcacgaagatgtttttcatcttttatagttgctggcaattcttccttaacttcttgcgccaggtaaacatttcacttccatgtaaaacttccgtcatcattgatgtagtatgagcaaagccattcgcaatcgttatcagcttcccaaatgcaaagcacatccatatcattaaactgctcaacgcttaactcaatgccacgcttaccagcgaagttagtagttgtttttgataccatcatttttaaattctccggttcgtttcgatgaatgtattatgccaaaacaaaccggatcgcttttaacaaaaagtgctatttagtgagttgatcaagccaatatctcattttttgccagcgtttaaaccattgcctatcttttgccagcttttgatctttacaatgaagcattgcatatagcaaatactctttttcgttttctattctctcaatgagtcggcggcacttgcccttgttagtaaatcggcgcaacagtttcattaatcactctccatgacaattacaggctccaggcgctcaagcaccactttgaaatcggcgtaaaaagtagggcggcgttttgttaattcctcatatgtatcagttgccaggcaagcgcgatcaccttcatagcacacaacgcgtttaccatttgcgcgactgattgcaactacgcgataagtattcttgttcatttcatcacctcaattgaataaacatgttcaacgcatggcgcgtatacacgatccccaaagtcatttacgcaaaggcaaagaggcaacccgcgattgtctgaaatatgcaagtgattttgtctatccatcaatatttcatattcgcgatcttcatcaaagcgccaaacgctcaacataccatcgcccaacattttgatttttgccttcttgcctacaaatttttgatagctcatttattcagcctccacagtttcaaaaacggtattgaatgccagaagtttaaatttcttgcctggctcaaccactgcgatattccaggtaatgccgccgtgatatgcaaccatatcatcaatatcgacggtgacttgtgcgcccttcttgaataaacctttctcatgagtagcgtcacgttcagtaactacagtttcgaattgcgtttgttgttcaaagttcatcgttgattctcctgattggttgatgtgtgtattatgccggaatcagatccggcgcgtttaacaaaaagtgctattattctcccacgataccaaagatagaaattattgagtttgtacgacttcggcgatatttgcgcataacaaccgttaagttttcgccttgctgatgacctaacgtgatcggcatttcagatccatcaccgaattcatcaccaatcttttttggcttttctcgccacgcgcaaatgaaaccaaatttatcacgcgtgatatagcggtatatgttaggaaggggaatcttaaccccttcaaagtctaccgtttgcgtgtaactgcggaaattaagcgtggaattcataaatcacctctccaaattctccgattacttgcgccttgtaatatcctccacgattagccaacaaatacgcctgactaagtgtttcaaacacgcgggttactggctggctgttttcgtgttcccaagtaatcaaagtagtcattgctaaatcctcatttcgtttcgatgggataactatatcaagttgccccgatccggttttaacaaaaagtgctgtttacagccattcgccggaaacaatgaaatcagtgaaatcgtcatgatccaaaatgctgtgataaccagtgcaataatcatttttaaccatgtgaccgcgcaaccgcacataatcagccttacgatccgtaatccagcgaataaagcgagcgttacgttgtggtatcgttccggcgtaagcctctttgaatccaatgtgtgcgatgaatgccacaaagcggatttgcaaccctggattatctttcttggcctcgttaaccaaatcgcggaagttttcacgcgttggcttgaccggaaccaattcgcctaactcgatcatgcggttgaagtctgaaacgctggaagatgattccataaacagcccatcgctaccaaacatcacaacgccgttttcgataaagtaagtgtgtccgttgtatttgttggtgtatttcatcgtgaattcctcagttcgtttcgatgaagtaatcatagcaattcgtgactgattggttttagcaaaaagtgctattcatgcgatagaccgattggtctagaccgtttggtctaatgagcacatcgttttctgtatcgaacggattcacgatcacgttatgcgatcacaatatgatcatgttgatgattgatgtgtgagttgatgtgatttgatttgagaggatttgattagatttgagtagcgagtgagtagaaataatcacacctcgccgatctcgtgaaaactttcccgcaaaacatcacaccctggcttgcgcgttgtctaaaacgttccacgatgttcacgtgtttaccttccgtaaaaaagcgaaacagaaaacaacaatgaatcaccatataacatactaaaaagtaagataattttatttatatatctataaaagtttctatttgtttagcttgttctctatatatagggataccccacgtatattttctaactttatatggttatgtgtatgcgccagggtatatgtatatatatgggtatgttccgaaattggcgtagtttttgaaaacgcgccgcaaaacattgaaagcaaagacaataaacacaaaaaggcgtctaaacagaacaatattctgattgtagacataagaacaacagaaaaaactttgactatgcgccagcacatcagcataatgcacacatcaaaaccaatcaggagaaaaccctatggaaaacatgagctttaccgaaaaactacgcgaggcagcaaaggcgcttgccagtggcgagaaaagcgaaatcgtgatcccaactggctacctggctactgaattagccggaaagaaatcatctgagaatccggcttcatacgtgcgcaccactatgtcacgagtgccggaagtaaaaatggttggcactatcaagatcaaaaagacaatgtgcgacgatgacgaaagtaaccttttcggtatggaagtattcaagatcacactcacgcatgagaagcggaaaactgtttatagcaaggctgacgttgaacggcacaagcgccaggcagtgaacaaggcagcagagcgctttatgacgctatcgccaaacctggcaaactatgcgccggaagaatacgcaaccgtagcaaaagtaatgcaagagcttcatgcacttgtgaaaatcaactttgtcaacaatgaggatgaataaa

SEQ ID NO: 6 - DNA, 41082 bp, unidentified
  source 1..41082: mol_type = genomic DNA
ctggtatacgcggagacacatccggcatctcattagctggctttactcgcggcttaggtttacgtttacgcttgtcttcttccttaggacgagtaaccttctttgttaccgccgatgaaggcggtgcaaggttaatgccggcgtgtttaaggatatcggtcgcgagactcatttctccccctgcttagataaccgttttagtgtacagataccgcgtacctgagcgtcaaccggacgctcgatgaaggttaacggcgcgttctccgcggcgcgtgcggctacctggcacgcctctaacgtgtaaaacgtttctgtcccggccagttgtagctgaccggcggacacgacccatatgaaaagtatgcttgtcatttgattaccctgtacttcacaatatcccagttgtcgttatcgattgaccacaggtaatcctgggcctcacctacggatgcaatgccgttgctgaattcgacctccaccttagaatcgcccggcaccgggcactccttacgtcctggattttctttccaaccttcggaagattcgtgtttatcgcgtgaagtcgcccggttgaagcccgcatccatatgctcaatagtttctcgtaactccttaaccttatctttcgcccggcataagcgggctttcagcacatgtatttcatcgtccttacgggcgaggtgctgtttgaggttatggatgatttgttcgctgtcagaatcccggcctatgtaggcgtcgataatcgcgttctccttgtctgagtaccctgagcggcctgttatcgaccggtctatgctcatcttgtgtatttcttctacaacatcgaacttacttacacgatgctgtagctgctcaatcaggtactcaagtacttcgtctttgttattcatttctcttagctcctctcgtttcgataaagtaatagtaccctattatgtttggttacgtcaatactattttacgattagtttatcctgacgcatcgtaaagtaacggggcttcttcgcatgagcgcgccaccactcaacgcgggcgtagatgccctgaagcgatgttactttgccgtgtctgatttctccgttccgcccggtccaggttacaaggtaatttggtttcattcgttagtctccgttatcgatgcgatgaaggtcatgagttccttgtaacaagcatcccacctcatagccgctgcttcatactcctcgtcgtcgaccctgattgtataggccatatccgccgatgcatcgtcgatatcatggacaagtttcatcagatatgttgcctgctctttagtaatcattgttgcgtctcccgcaatagttacgtttattgttgcatttcgtgaaatagaaaaatattaataaaaatggtacgctgctactcagcagcgtaagtcactcagtcaattcctaaatagttgttacaatataacatcacacttccggtggttccggtagcggcatccgctcactacatttaatccacttactcataactccacctttatcgcaaaaacttccacgggttcactgccgaaatgcgggtgtgtgatttcttttatctcgtacccacggtacttacactctattctcctgctcgcgtcatcccgtttagggtaacccttcgttataatcaaacggtcgaagtcacggccaaccaggcgcgtccaccaataatcatttaccagacgatactcctcggtcttctccccgctcttaatagcgtcaaaatattcacttttcacggccaactgtaagtttttcacaaacggcaatctcctttaagattcacgacccgaagacacagactcttcagaatcttttgccgtgtagttattccctattagtctgacgaagcgctggctcatccacacgatatgcccgttacttaaattccctacatagagcaacgacccgtgctggtgcagcactacgtaatactccatagagatggtacaaattaccaactgacccttcttaaacatgattcatcctcttgtcccgttaaccttgcaataatagtactctattatactgggttacacatgtgcctttttattcttctctctcgtccctcgtcaggtaagcgcttgccgtaaggcaagtgcgttaatccctgagtagtagaccaagaatctttcggcatttcagtaacttaaaggaatagctgccgatgagaatatagctcgcccaaggcagttcagatactaaggaaggaagttacttaattttaagtttgcgggtttcagtaagccttaacgcgtttccgcttggggcggaaccgctaagctctcacaaggtgaaacagaaacttaagtaacttgaagttacaagagacaacttacacgaaaaaaatgaaaatgtcaataggtgtgtttatagttgatttcccattattccgtgtgtatacttgctttactaactgagatggaggatttgagaatgacacagaatgaagtagctgagcttattggggttacccgccgcacgctgaataactggttaagagacggcaagttcccggattgttgtgtccggattatgggtcgccggatgccaggcacatttgaccgggagaaagtggaagcgtggattagggagaatgtaaagtgaccgatttccagaaaagggtattcagaactgtagtagcgcttacatgtaaaaaagggtcgtgtagtgtccttgacttgcgccggacttattttcagtactatagttcctctctcattgagaacacgttgaaggttctggttagagacggagcagttaaaagtaaggccggaaagtattcggcagtagctgaagttaatggctcccaggcgaccccagaagaccttgaataaaagaaaagccccaacgcgcgaacgtcagggccttaaacactacaaaggaaaagcacatgtcaacaggcaatttagcatactcatggtcgcgtcgcaatcgccgtgaggaacgcgctgacattaacgtctacaccacgacttccggcacctgggatgagttcgttgaacttatgcagccgcttaaacgttcgcgccggaaccccaagacagaccccggatatatcaccgccgcgtgtaccgccacggtaagctctactggtaaagaagccgccgaaggtatgttctatcgctgcaatgcgtctgttacgtcttcatcactagcctatgccgacgtggacagcgcgacgccggaagagttcgccaccgactgtgagatggtgcgcgaatcacgtttcgcaatgatgctctacaccacggcatcccacaccgaagaagcgccgcgctatcgcgtcgttatgccggtgcgcactccggtaaccggcggcgacatcattcgcatccggtacggcctgttggcacacttccttaaagggcgtgatgtggatagcgccgggttcaccctgtcgcagcctatgtaccgcccgccggtaggaagccaggtcatcgtgtctgaaagcggtcggatgattacggcgagcaagcttatggagggagttcctgaaattaacattacgggcgcttccgattataaagtgccggatggtgagcaatccgaattaaccgacctgttcgaagaattcgcctttgagttcggcggccgtattaccgaccgcggcctgcaaatgcccgccacgccggagcacgccgcccaatacactaccggcgaacctaagcaggatgacttcctgttctgttggccgcgcgacggcttcgaacgccctaatgtcaccctgtaccatgacaccgacctggtagctacgggcgggatgaagcctggtggacgggatatgtgggcttacgcttgcgccgctaccggcctgccgttcgaccgcgtagaggtggcgcttgggtgggcggagggggtcacttgcgacgaagaggacctggccgacgaagaaccaccagcaccacaagcggacttcatcgtcgaagggtacatgccgtctaattgcatatgggatatcgtcggtgaatcagggacgtataaatccttctacacactcggcatgatgtatatcagtgccgcaggataccggttcgctggggcggatacccgtaaggcacaccacttctacatcgatggtgagggtggggagttcacgcacacccgtatcgctgcgttggccgctaaatacggcgatgaaggtatgcggtatatgcatgtgcttgacgctggtgaattcgcggatacgaagaaactggtgcgtaaaatgcgccagatagcgggcggtgaacctgtcgggatggtcgctttcgacactcttaaccagaccttcgggaactggatagacaagttcaacgagaactccgccggtcaggatggtatgggccgtgtcgtggctatgcttaaagaagtacgcgacggtactaaaggggcggtaggtgttgtccaccacaccccgaaaggagggagtaaggcgcgtggaagtggtgcgttgtatgccggtgttgacgtggaactgacacttgttcgcgccaccgagaaacaaataaacgtcgcgcacaccaagaacaaaaacggtatgcagcagaaaacaataggtatggtgcttgaaccggtacagtttcgcgaagctccgccgccgaaagagttccaggctgtagaattcgttgggggtgagggatacggggaaatcgttaacctcgacctgccggaaccacataaagctctcgtacttatgccgtggggcttccagccgttcgaaacggacgaggagaaagagcgcaacgaaggactcgatggtaaaggtaaagattccgtaaaggatactgttaaacggtctaaagatgcttccgcccgcgaatcagttatgtctgctctcgaagatttacagcaagccgatgatacaggacgtggcttcacacaacggcaaatagtcgcaagagcgggggaccacagcatcacaaacctggtacttgagaagatgctacgtgagggtgaattgatgcttggttgcgatgaaaacggtgaagttgtaacgaatacttacaggttgccaaccggtatagacgaccgaaagcggccaaagaaccgatatgaacctaacgacaacataaagacaacagaaggtgatttagagtaaaaagaaaggggccatcaggcccctgtatcaccaaaacttaaccatctagggtcgacaccgagggcatcggcaatatgaaagattttaaaacagtctaccttagatacccgaccggttgccaggtgggtaatcaggctttgggatacaccagctttgaaggcaagctcggtctggcccatgccgatttctttacggcggcgctctacacgagcgcctaattcggattgctgcatatttttactccttagttagatatgtagtaatagtacagtaataaaatacacgacgcaatttacgaaaaagtgcttgcataaataatagagcacgattatagttcttatcaccaactaaacaggagaacaaataatgttagagaaattcttggtattacttgaacgtttcgtagttgcccatgaactgattgctgcgaatagcgcaaaacagatacctaacaaatccgtaagcgaaatgactgttgacgtctccgttgcaggtgtcgagacagttaaaaaagaactggctaaagcactggaagaagtcgagaaagcaaaagaacttccggtagaaggtgaagacgtcgttgacactaaaccggcggaagaggagaagccaaaacgtaagccacgtaaggctaaagcagaggaacctgcgccggaaccagaagaagagaaggaagaaatcgattaccagtctcttcgcgaccagattcagggtatcgatgacgcaattggtgagggcccgagcgatgccgcgtgtcatgattccgatgacctgctggaagagttcaccggtaagaagatgaagattgccgcgattaaagacgaagacctggctgaatacctggaacgcatcacggcaatcaagaacaagtatttcgaagaagaataattacccggcggcccttcgggccactttaactgagggttaacggatgaagagtaaagtacttatagtgactatagaaacagcttgggtcggagcttcagaagttgtagagatagaattagatgggtctgagactgaagaagaattagaagatatggctaaagaagagttcggtaactattgcaactacggttggagggtagaagaatgattcttagagaacgcgccggcaataacgatgtacacgccttattgtcaccatccggtgctaagaaatggttaacatgccctgcgtcgctgatatgcgaaaaagatatccctaacacttccggcaaggctgcagtgttaggtacggcaatgcatagcctttctgaatatcatttaaacgcgtatatacgcggcactgagctgccgttagagcgcgacatcggtgcgtatgtactggaagaaggtaaaggcgcagttaaagcactaattaagccgatgaaaggtgcggttcttattacggatgatatggtcgaacaggtgcgaaagtacactgactactgtaaagcggttatcgatgtagcgacctacgccaagcttgaaatgcgcgttaatcttagtgaggtgttgcaccccggatacgaaggcgttgagacgttcgggaccgctgaccttgttgccgtacaggaattggctaacactgacgatcacatgctcgttattggcgacctgaaaacagggcgccatagagtagaggcgaaagaaaacaagcaacttatgctctatgctctcggtgtttaccgccgcctaaagagacgctataacataacagctgttcgtctggttatctttcaaccgtacaccggtggcgcgtcggagtgggatatctcagttgaaggtcttgaactattcgctaagttcgcgcagaaacgtgcggtagccgcacttgacgcgtatcgacgtggtaagaaaaaccttaaaccatcggacttcaagccgtcggttgatggttgccaatggtgccgtttctcagaacaatgtgccgcgcgtacaaaaaccgttaattctgtactggcggaagaactggaagacgatacggatgtatatcgtacagccgtagaacgtgtagctaagttctgcaacgacgcggactatatgagcgtttgggaggatgttcagccgagcgatattcttaagttgctgcctgatggcccagatttacctaagaagccagtgagggaggacgactttgcactggagcttacgccggagcaactcgtcgctgagtatgagaagttgccgttgttgcgccagcacatcgacaaagtagaaaaagcgatggcggccgcgcttcattccggtaaaaaagtgccggggtacaaactggtcgagagccgtccgggtaatcgcacgtggaaagatacagatgctgtaaataaatttagggaaaaccatgtaaacggctatctgctggataagactgttccggtatcaccgaccgaagcagaaaaggttattggcaaggaagacccagaactttgggccgagctagctaaactcataacccgtaagccgggggcgccatgtgtcgcaacggatgacgacaaacgaccggaatggcaaaatgttacggaggaggatttgtaatgaagaaatttacatggtggttgctagcagtcccgctaataccgatggcttttatatactatttaatgagagcgttagagaaagttggtgaaacgtcggcaattttagcggacaggatgctagactcaaaaatgaggatgaaatatttagcatggcgtaatcgaaaattcggttgacaagttaatagtactctattatattcttaatcactgaccgggcagcttcccggataaactctaaaatgcgagaaaactaaaatgggaatcaaacttaatcttcgtaaagtacaaactgcatggttgaacgtattcgaacgcgctaaagaccgtgaaaataatgacggttccattactaaaggtacttataacggtacttttatcctgactccggaacacccgcaaattgaagaacttcgtgataccgtattcgctgtagtgtcggaagcattgggagaggccgccgccgagaaatggatgaagcagaactacggcgaaggtaagcacatggacaagtgcgctgtgcgcgacatcgctgagcgcgataatccgttcgaagacttcccagaaggtttttacttccaggcgaagaacaagcaacaaccattaatcctgacttctgttaagggtgaaaagcaggtagaacctgacttcaatattgatggtgagcagattgaaggtgagcaggtatatagcggttgtgtggctaatatctcaatcgaaatctggttctccgagcaatataaagttttaggcgcaaaactaaacggcatcaaatttgccggtgaaggtaaggcatttggtggttctgcggtttctgccagcgtcgacgacctggaagacgatgaagacgaaacgccacgtcgggaacgtcgtcgtaaccgttaataacttttcactctaatttaggcggcttcggtcgccttttctataagggtcaaatgaatgaacctgctttatcttgatactgaaacattttcagaagccgatttgaaaaaagtcggttcctatgcttacgccgaacatccgactaccgaaattgttatctgcacctacgctttcgatgaaggccctgtgcaagtatgggacgccaccgacggcagcgatatgccgcgtgatttgcgtcgggcgatgctaaagctgcaaaaaccagacagcaatctcaaactggtaggccaaaacttccttatgttcgaccgaccagttattaagcattgctggggattcgaactcctggtagaaaacattatagacactatgatagtcgcgttccgacatgccctcccaggttcactggccgcgctgtgtgaggttttaaacattgacgcaagcatggctaaggataaacgcggtaaggcgctgatacagcgattcagtaagcctacgcccaagaactataagattcgacgttatactgccgatacccacccaaaagagtgggcagaatttattgcatacgcaaaaagcgacattacgtccatgcgtgaagtgtataagaaaatgcctaagtgggggaattctgagttcgaagaccgcgtgcttcatctggaccaggtaattaatgaccgtggatttaaggtcgatgttgccctcgccgagagtgcaattgaagcggtgacgcgccataaagaagagttacaggaagaagcccaacgcaaatatggcggttcactaaccggcaaagacttcctgcctattttacaggaactagcaccggcacaccgcatccacaacgcacagaagtcaacattaaacgacctgctggccgatgaggatttaccggacgacgcccgcatgatcattgaaatgcgtcttggggccgcatccactgcatcaactaaatacgcaccgttgctgttaggtcgttcttccgatgaccgccgtcgaggttgtctgcaatacggcggagcgaagcgaacactgcgatgggccgggaaaggttttcagcctcagaacctggcgcgcggatattatcacgacgatgaacttgataagggtatttccgcattacttaaaggccgtgcacaccgccgttttgatgtggccaagctaacggcatccactgtccgtagctgtataatcccggaagacgggtgtaagtttgttgtcgcggactattccaacgttgaagggcgagggcttgccttcctgtccggggaagaaacagctcttgatactttccgcgcgggtcttgatatatactgcgtaacggcaggaaagatgttcggcatggacccagatgatattaagaaaaactttaaggatatcagacagataggaaaagcttgcgagcttggcctcggctatgagggtggcgtcggagcgttcgttacgttcgccaaaaacttaggtcttaaccttatcgaaatggctaaaacaatggccgggactttccccgaccatatctggaccgctaccgctcgcgggtatgagtgggcgcggatacaggaggcaaaacggccgccgcggccaggtgaaaaggatgaccgcccatcgtatattttggacaagaaagtatggcgtacatgtgatgctattaaacgcatgtggcgggaatcacacccgcaaaccgtagctttctggcgcgaccttaaagacggtatccttgccgcggtacgtaatcccggtcgcgatttctgggcgggcgcaaatatccgaagtaacggtgaaagagctatacgcatatggcggacgacagaaaccgattcatccgggagaaacatcccgggatggtggctatgcatggagctgccgtcggggcgaatcctatcatatccaggaataggcgtcagcgtgacaaaggaaaccgacgaagacggacgggtaaacaccaatgtaaggattaagtaccagggggagaaccagttaacgcggcaatggactaccctgtatacccatggcgggaaggcttgtgagaacatagttcaggcattttgtcgtgatttattggcctacgcaatgcttaatgtcgaagctggggggtatcctatagttctttcggttcatgatgaactggtatgcgagaccccggatacgccggattacacggtagcggaactggaaaagctaatgtgtgcattgccgggatgggccgatggtttccctcttgtagcggaaggtgcggagttaaaacggtatgctaagtaaactaattatctattatttccgtgaagaagattgtcgaatctgcccgcgctgcggtattgaacacacgaaacgcgaggggtgcatagatgagtacgcctgagggcaagattcagaaatacgcaaaagagaggttcgaggccctggggggcctcgtacgcaaattgtcgtacgaaaatagagctggcgctcctgacctgctggtaattctccccggcggcatcatctggttcgtcgaggtgaagaaagacgaaaacacgaagcctgacccgcaccagctacgggagcacgagaggatgcgcaaacgcggtgcgaatgtttttgttgttgggtcgaagaaacaggttgataaattaatagaacactattatatttagttcacaccaaacagaaataaggaattgagaaatgaaatacgaatatgcccgcaaaccagcacgcgacatcgtaccgggcgatatgattttcaacgttaagacacgtagccctgttgccgtagatacagtatttgttgagtcaaacggtaaactggttatcgaagatgtaactggtaacgtcacggcgttcgggcggaaggaattggttctggtggcgaaatgagtaaattcactagaaggccttatcaaaagctcatgacctcgttcatgctgcgacacccgcgctgtaatatctgggcaagtatgggcagcggcaagtgcctgaaacgcgggactgaagttattatgtttgacggtacgaccaagaaagtagaagacgtcatcgtcggtgacgtgcttatgggaccagattcaacgcctagaaatgtactgtctctcggtagaggccgggagatgatgtacgaggttaaaccgcgaaaaggcgagtcgtatacggtaaacgaaagccatatattatcactgcgaactactacaggtatcgcaaaaggttcatggccagataacaccgttttcgacataagcgtccgcgactggcttaagttgccgaaatatgtaaccgggcctaatgggtatcttaagggttggcgggtacctgtcgattttccaaggaaagagcaggacgaggctttattgcccccgtaccttatggggatatggctgggcgacggtacttcgtcgtctggggccataacatcaggagagaacgaaaaggaaataagggcgtatcttgagtcgtacgcagcacgtaatgggatgcaaatacgaaaagaaggacttacatggagtatatctcacggcaacacaggtcataagaagcacgggtttacgcacgcgctgaaatcggcgggtgtcctgaacaataagcatataccgcacaattacaaatgcggcgaccggagacagcgtttagagttgctggcagggcttctggatagtgatggttactgcgatttgtctaaagcaggttttgactggatttcggtaagcgagcgactcgctgatgatttttgctatctatgccgttctcttggtttcgccgcttacaagaagaaaactcgtaagcgttgcgcaaacactgatgtttggggggattacttccgcgtatcggtatcaggcgatttttctgatgtacctttcgtgcgtggccgccaccagaatttaccgaagcgtaatattaataaaaacgtacttaacgttggaatagaatctataaccccggtcggcgtagatgattattacggcttcactatcgacggtgaccaccggtttctattaggcgacttcaccgtaacgcacaacacaagctcgacaatgtgggtgcttaaccggttattccgtaatggtcagcttaatgatgacgaccgcgtgttaatcctcgccccgttgcgcgtcgcgtcgggtacgtggccggcggaacaagagaaatggggtttcccgtgccttcgtgtcatcgatgctacgggttcagagaagcgccgcatagcggcgctcgagtcagacgctaacgtggtgtgcacgaactacgaagttatcgagtggctgattgactactacggcaaagatgactggccttttacggttatcgtggccgatgagagcacgaaactgaaatcattccgtagccgttccggtggcagcaagagagcgaaggcgcttagtaaagtggcgttcggcaaggttaagcgtttcattaacctgaccggtacaccgtcgccgaacggcctaaaagacttatggggccagaactggtttatcgatgccggcgagcgcctggggtcttcgtacacagcattcactgaccgctggtttaactcagtacagaaagggaagtcagctatggcacgagagtaccacgcccgccccggcgcggataacgagattcaccagaagatgaaggatatcagccttaccattgatgccgccgagtggttcggatgtgaagcgccaattattgtgccggttgaaatcgacctgccgaagaaagcgcgtcaagcctacatcgatatggaggaaaagttattcgcggaactggagagcggagaagttgaagcggctaacgccgccgctaaaacggctaagtgcttgcagattgcttccggtgccgtgtatgtgtcagggccggatggtgaagcaacgaaagactgggagaaagtgcacgacgcgaaactcgatgcgttagagtccattgtcgaggagttgcagggtgcgccgctgctggtggcctatcagtttaagcacgaacttgagcgcattcttaggcgattcccccaggcgcaggcgtttgcgaaaggcgctaagggcaataagcagatggaagcgtggaaccgtggtgaaatacctatgctcctgatacacccagcctccgcgggccacggactgtcgttacaacacggcgggcaccacatggcctttatgagtcaggggtggaacctggaacattatttgcaggttatcgagcgtataggcccggtaaggcagatgcaatcaggatacaaacggtctgtattcatttataaaataatagcccgagatactcttgatgaagttgtcgcagcgcgtacagacgagaaaaggtctgtacaggatgaactacttaattatatgaagagacgaggtaagaaatgaacatcatagccccgattccggcattgcaaaaacgtattaaggaactcgaagaagatgttctacgactatgtcagcaaagagacgccgctaatgcgcaacttgactttattatggagaacctgaaatgcgatacactaccggagcaaaatgcttcatagcaggacttattattgccggtgtttttactatgtacgctttgtacaacgtaatttgaagaaggcccctttcggggcctttactatttatgccaaagttaatcttgtatagcttccatcggcccgcttaactaacgccttaaggctatcgccttccagatacagtgatatagccccgttatccctgacagcgttgtctggtagggtggacaccgctaccggtatctcaacagcacgataatctcccctgtttatttttaatgacacggcatcaggcaaggaccccgaaatagcggtaatttcggtccatgccgaaccagaacctggcccgccgttaatgtgggagtacacggacccactatcaagggtttttgacagtttatggatacgaaaccgaagcgccgcagaaccattattgaaactgtttatgcgagagttaccaagaccttcctccatcaaattggctacgttaatccttgaggggtctacgaagccggttataccgctcacagtggagttaggcgcgtctatcgtaaggccctgttcggtagttgaccggatgccgaccaagcgcagcccattaacacggcaagcccctgaaatgtaaatctgattagcaggaaagtctttagtattcgtgtctattaccgcaatgtttgtgaatactgtttcatgcgtcaacagatatgctccactgccagcgcaatcttctacggttatgttagaaacgtatagcccttgcccatccatgtcgaagccaactcctaaacaccctctaaccaacaagttatcaatcaaatggtttaacgggagcatatgtactgggtactgagatacagagtaatcccccggcctatctgcttccgggttcatgtcggtatctgcgcctaaatcgaatccgtcccataccgggtacaaaactacggaatcgcggaattgaagattataatttcgtgacgttgtggaacctacagtcccctgccatgtcttaactccgctctcgccggcacggtatgaggtaaacccaataaccccaccatcacgttcaaagccaccattattacgtaaaaactgggcgctacttaccgaaccgtaacttgtacgcccgccaatgacatagtttcctttaccccaatccccgctcaggttttcaaaggtaattacgccgtcttttccgccgctcgggttgtcagcgtctaccatcttacagaaatggcatccgcggaacaggaaacacgccataagaccactcgcccggtgaacctcgacgcctgtacattcccgtatctcaagaacagacgaaatgttctggtctttagcttccggagggagaagggattctatacccgggaacttggcgtaatcgtttaccgtaggctggtatccatctgttttagactgtttaagtgtggccacgattgctgcggggtcggttatccactgattatcgtcagtccacggtttaatcacccacggcgttgtgacactctccatgtaagcaccaactacaacggaccctctaccaagttgtgtgaaaacaaggttaccgtcgccaataaacttcgctttacagtcgatggttagggtcttaccgccgaagttaacggtttcgttattgctgaaattgtagtcacggtcgataagaacgctgtcgacggcagcatcagctaattgctgtaaagtggtaaaatccgacaacttaacggagtatttaaatttcttatccgcttctgcgcgaaaggtgatatccccgaacgcgacccaagcggaatccgaaatgccacctgtcgtaagaggtgtcgacgccgcagggataactttagggagagacccgcgccatgcgtaatagttaccgtctccgccgtcttcaataggccaaagaaccgctttatccgcgtcgttaacccctagcgtcccgccggttgtgaagttgaaagatgctggggagaaaccagcatcacgtaaaaccgcgggcagggtcttctgcgtctgcccggttacctggttagtagcgtagtcgatatctgcgccacccgctacaccaccctgtttgccggttatcacctcggcttcgaaaatctggtgttttttagctacctgtaaatcgttaagtgacaatacatcaccgcaaccactagacatatgactatcctcttagttaaaaccgttatcgaatccgttagagaacgcacgaccgaaaggcggtacgttgtcgaacttatagaaatccttgtcatagttaaacccggttattttgactgttctgtcatcgccggggtctactgtagaaacaagaatcatctgagcattatgcctcgcttcgttgccgaatgaaaattcagttttcagggcgctattacccgtataaatggcttcctgcggtacagaagtcataattacttgtcggtcatgcgcaccagggactacacgaacactttggacgccaccgtctctcagtttaagaaccaaagaatggtcatccccggaggtgaacttaactggctgtgacagctccacggtgaggccgttcaccgcagttacgtagccgtcgtaggtagccatacgggaacccttaaccacactaacggcacgatttgggagggcaaagatcccttcttcagtggcgctaaaagacactgaaattttctttaaaatgttcttctggtggcgacggttcgctgcccagaaagcctgcttatagttgcgaataccttttgaatcgtaggtctccgttttaaccccaccagtttctggtattgttattgtttctttaacgtttgttttagggtctatgtatgagaatttaagactatcgaacacctgagcatcgttaaaggttctggtccatttttcagaagtaccggccttgcttcggtgggtgaacaccatctcggggcccatgcgaggtctttcaaaatcaagaaggatatccgctcctctccggtatggggtgcagaaaatagcgtccgctatagtactaactatgtcttgcatagtggttttgtagtcatcgaacgtgtagcaaaattcccctgcttgcttgtcaccgaaataatcttcaatctctttctgaacggcaagtaacttatccatgtttcgcactgtcaggtttaagccaccaacatctgggtcacgtgccaggcggataagagattgcacggcctgggtattaggggtcatcgtgtcttcgaaaacaccattaccgaggtatttgtacaccatttcggtggcaatcatacgtaactgtggttgcttaacctccgcagcgcgcggcgtctgcttgcgcatcgagtgtacggttgttctgttgccgtagtgcggagtgttatcacgtgtttgtccgtataagttaacgtacgttatttcatcaaccacagacccctcgaaattaaagtcgaggtctgtcacccttctggcgcgcacccgcacccgcgacacaaccggcaggtcggcgtaaatagtgacaccgttgtagtctggggttcgcccggacacggttccttgtgcagtgtatatcggcccataaggggtgctattttcatcaagcagctggtactgtatctccgcggtaaccgacgctagtgttttagatttgccgttgtctttatacataccattggcggcggcgatattggcaactatgcgctcaacctcggtacggtttattgacacccaatcggttaaggttttctcgtatgtgttatccggttgcagcgacgcctctccgtcacctcgaaaaaatgaacctggtcttatttcttgccaccgaccgataaggccgccaggtacaataaatgtaattgtcccttcggatacgcttaacacttggtacccacccccggacaagtcgacgtctgtatttgtccacgctgacaccaacgaaaacgtgtcattaggtaccaacactccagaaaaatccgaaccacctgttgggtcatatattgtgccaatgttcccggacaaagacgggtacccggacatgtaagagaaagaaatacccaggccgttgggagctttaagaaccacgccatcgacttcgttagattctacggtaatgtacaggccctgctctatagggtcgccgaccatgacctggggtgccgatgtgttattgggcgatgtatatggtgagtacacggcaaccgacgtgcctgttatgtctgatacgcgggtgtccccgtcagttataccttcagggtgtatgtctaggtaaccacgcccggcgtcgtaatagccgtactctacaattttaccagcggcgttaaacaccttataagtagacataaggttatttgggatagtttgcaccgttccgcagatgtcgtagctacgctcgtatgggcgggccttgttgttacggtcggtgagactgttgttcggggaatcagcctgcgagttcgcaaggttagatgacgcccctttcacggatggtgataatagtttcgctagcggtttaaggacaacgctaaaaatcttcatgacgcctttaatagcgccaccaccggcagattccacaatgtggaaagtcgcgttttctttcagtgcttcgaagtcttctgttacgtcgttatcttctccgatttcgtcaatgaaaacacgtaccggaaccccatccggaacatggttcacgacgaagttcattggattatcgtgataacgtttaacgtcaaatgtgccgtcttcgttgcgggtatagtggattactagcgccaaaattcaatctccgaataagtatctttaaggtcttccagactatccagtctaacctgtctggacgccagctcacagtggcttaccattccttcgtaatataccccagcatgccacactattcgccccctatgtttcactcctagtagaaccgcgtcaaaattctgcggtgtgaccgctcgtaccagacctttcggattagagtggccatcgtcgaaggcggcgtctattgccgttgggcttgttacatcgaacatcggggtcgataaccccgcgtcagcccggacgttgcggacgtggtgccaacaattacggcggcggaagtcatacggcaggccagtgtaatcgttaatattcatgacgtcagtatcccgcggagtaagggtatttcttccggcgtcatcaggattccggtagctcgttggttaagcatgggtgtacctgtttccgctgtgaacactcctttctcttgcgtgagtgtttgcaattcatataccaccggcccatcgcatgggtatgacaggtccgtgctcacgtaacgccggaagatgaatttgggcaattccttattatcataagggatgcggcccatttcctcgtccagtatattaagcacatccggcaaagagaaagaggcggtctggtccatgtcgttattgttggcggcgttcttggcctccatcggcgtaccctcgaatgtaacaacctcacccgtctctagagtagcggtcaagtcgttagccccgcgcacaagcaaccagcgcttcgaaagaagagggtgggatatctccagggtaataaaatccatctcgccatcgggattggacgccagcttacgccggtatgccgcttctactgattcctgactcattgcattgggtcccaaattcgagggaatgtagtttggtacactccgtaagtttttagaaattcgccaagacaatcgccgtagcatccatataagtcgggcagattctgggtaagacaggagctgcctacttcctggaatggcgatttctcggctgtggccgtgaaagttattacccagttcttcccgtcgtcggtggactcgttccacgtagatgttagagtaacctggtaatcctcgataccctgacccaagtcgtgtttcatccagaaactggaagcccctccgtctaccttctccatgaaactgaggaatgcttgtctacccagtggcgacacgaccagcgtaacgctaatcgggaacacatcaaagtatgtatcacgcccctgtcgaaccccgccaccggacaggtctactcgccacacgttgttacctcgcgtcatcgagtagcctttcgatactataggcctcaaagaggccgggaaatggtaatcgctcatatcaatatcctggttggccgcgtgtagcgcgacgtgatttagaaatggcgctgttactatcctgtagcgcagaacttacggtttcactgattataatacgtaaacgaccttcgtcgtcgcgttctgttgcagccgaatcaattcttccagtggtgttgttcacgatagtaacattatctccaccggatttagcgccgttctcacccataatctgtcgcatctgctccgcagtgcggacacgtgacgcgctagctggcatgattacttccggcttaccgcgttcagcgatagtagacatctgccctgcggcgaggttaccgccctgctcgcgcgcggagcgaatcttacccacgttggcgagacctgcggctacggcagcagcagcggcaaccggtgctaaatatggccctactactgggatggccgccgttgatttatacgcttctactgctgacgtgtacgtagcgattgtcgcctgtgcgatggcaaatgccttgtacgcggtggacgcttcccccagtgctgacccgatgttgctcgccatgctaccgaaagccgcgccggttgccttcgcccggtcaagggagtaagactcatccatagcgcttaacgtttgctggtaggtttcttcgccaatcaaaccctgcgcatgaaaatcgttaagtttttgtagcttgacctcgtactggcggtctaactccgcgaactctccttcagcaatggcctgcatctgagcgacatacgcatctgcggagaattgtttttccaggcgctctttctcgcgtttatctaactcagcctggcgggccgtatccgcctcgagcataatctgcgtcttagcggtttcgtattgctggtcactaaggagacggttgctgtagaactcctgtagcttagtaagttgctcttcttggttagcttgtatcttctggaatacgtcgtcgttaaggtggattgactgataatagaaatcctcagcggctttcttctgacgttcgaatgcgtcgacggatttcttagcagcattgtccgcggaatcgcctacagaaccgttgtctttacccgacactttaaacttaccaagaccctcggccttggccgcttctttatcgatgtcgtaagccgcgcgcttcaatgcgataaggtcttcctgagcttctacctgttcacggaaacttttacgtgtcaggtcagtcacagctctttgggtatccatagcaagcgctaactggaagttaaggttcttggcctcagctaccgactctcccggcgccaatgcggcctttacaagacgtcctgcatcctgagcgcctttaaccatatcttcaaaaaaccgctccattgccgtaaattcctgtgcaatgaagtccagcgcatcagccgctgattcggctattgttgacgcggtctcctcaccacttccggtaatgctttcactagtgtaagtccaggcggagttcaaagcgtttaacgcgtctgcaaccgtcccaaacgccgcgtcgaatgaagcctctatcgcacccccaacatcggatgccatgtcaccccacatctgcatctcggctacgaactcaccggatgcgacctgagcgtttatctcctgtatcaggtcgtcaatataacgtaaaggttccgccagcgatcctacatccagaccagtagcgagagtcatctctaactgggaccatgagtcctccgcactagcgatagccccgttcagggtattagcctggtcagccatcgccccggcgaagttaacgttgcctatgttgaggagatatcgctcaatatcggcagcgtttttcttaactacagtagtcgtcccctggaaggtaaattcgatatcactttggttctgcttggctttaatgccgaattctttcagacgttcgaattcaaaggtgctcgcatccgcgacggcctcaatcatctggttaaggtctttacccatcgcggatgcagtattgccgtaagagcggagcgcttcttctgatggagtgaggcctagagctaccagtttacggaacccttccactgcttgttcaaggccgtaaggtgtgtcacgggcgaagtcctgtaaaatactcagggcctgttttgcgccttgagtactgcctgtgagcgttttcaggctagccgacatcttatccagttgccgttgcgactcgactagttcctgtgccgccttataggccgcagcggctacagcggcagccagtccggtgaccgcggcccccgcaacttttgcagatttagagaggttatcaagacggtctgatgtggccttcgcgccttgttcggttacctttactactaagctagctacatcagccatcgtttctaccctcaaaaatggcgtctaagcccatgataatttcagattcaaacatccctatattctgacctgaaatagtgctatacgccactaaatctgaccaactaagttgctctcttgggtatagctttacgccgtcgtcatcaacgcggcgggtgaatttaacatcacgatacttctcaaaagtggtgagtagttcaggggggcattcaggcccggcgtcctgcgttgtttctcccgcgtcttttattacccccatagcgataagcgcggctttgtgcccatcggcgatactgtcaaacttgcgcctttcgtggcgggttatgaagttccaacgggcaaactgaaacaacgcgtctacttttcctgcaattgacgcaactgttcgtggtggaatacgactacatgttccgccaggcgcttatattgggtgagaagagtcttaagattttccttagtgaactcatcatcgagactccagccattcaccaattcgagagccaattgtcggtttaggtctcctgccgcgtcttccatttttaaattgtattctgagaagtctttttgctcttcgcatttatcccgaagcggctttaatttacctaccgccgctcggtacgccagggtgaaggcacgcatagctttaaccgcgacatcggcctccggggagacaacgttcaaccattctcctgaatctgtaccatcttttaaagggatcggcatgcgcgcgcctttctcggcttcggcttcgtagtaaaaatcgctaagtttcatagtaatgccctttggttagtcggttactggttggtagttgcaccaggcgggaaccacccgcttttccggtgcgaccgtagacgccaaataagaataacaaaatagcttgcataaccgttaatagtgtcctattatctctacgtcaactaaacaatatgaggttaagaacatgagcgtttttatcggtattatcgcgtttatctatttcattccgttcctggtagccttgctacgtaatcataaagcgaaattaggaatattcgtatgcaatcttttactggggtggattcttctgccgtgggttggggctctcatctgggcctgtaactctaatgttaatggtaaataatctggcttaatcaaaattacattattagaatcaggtttgtcttaagcaatcggggggggggaataagcatggtaactcgtaaaataacagatgaacagttgcagcaagagctaaacgctggtctcgggccgaccgaaatcgcaaagaagtacaacatgtcacgccgtaatgttcagctaaggtcagctcgtctggccaagaaaggtgttggtcacggtcgcgacgtaagccatctggtgccggatgggtataaaataaagggtacgtcgtcactggtggatgagttcggcaatacgaaacttcagtgggttaagactgacgccgacgccgagcgtcaggtcgagttgatgcgagccgtaatagatgggatgaagtccgatattacgccagtttcttcggtccctcggcctaaaaagcggctaaacgaaaagttgctaaatctttacactgtttctgactttcatttaggcatgttggcctgggcggacgagagcggcgatgactgggacatgaagattgcagaagaccttttctctaaatggttcgacgcggcgtttcagaaggcgcctgatgccggtgtgggtgttattaacctattaggggactttgcgcattttgacagccttgatgccgttacgcctgccagtggccatgtactggacgcggacacgcgttaccagaaactggtgcgctacatgattcgtatggttcggcgtgtggttaacatggcgctggttaagcataaatcagtgcgccttcttatcgtgcaaggaaaccacgacgaatccggcatgatttggttggctgagatgttcaacacactgtacgacaacgaaccgcgtgtatttgtggatacgtctccggatgtgtacaagatggtgcagcacggcaaaacaacactgttctttcaccacggccataaagctagattcgacgctatcgaaccggttatgatcgccaagttccgcaaagcgttcggggaaagcgtgtacagttacgcacacgtaggtcacttacaccaccagaagattgtagaaagccgtaacatggttgttgagcaacaccgcaccctggcggctaaagacgcctatgcctcccgtggcggatggatgtcaggccgtagtgcaaatgtaattacttatagcgccgaacatggcgaggttgcccgcctaactatttctccggagatgctgggatgacaaataaatacaaccgtacaatgacaaacactgaaggtgacagcattacctgtgatgtgtacgacgttctgagagcctttgatatccgcgaccctgcactacagcacgcattgaagaaactgttgtgtactggtttacgggggcataaagatgctgatactgacctacgcgaggcgatggagagtctggataaataccgactatatctgagtaatcttgaggaatgaaactaaggccccgaaaggggcctttttattaagcatacttaatgcgctggataacaattgaagactggaaccggttaccggttgcctggccctcaagagaaagggtaatagactccgggccgccgatttctggggttgcggacgtaagtgaagcacgtttgagtgtaaagctcatagcgccatccggaccatcaagaaccgaagatacctccatttctgtttcatccaggaatttatttaacagcgtcaggtcgtacagtttacctgccagcgagaaggtgttggccgcacgactgcgctcaacaaatgccacgctgttatttcccaactcgaactgtgcggatgccccgttgtcgttagtaatagtgaacgtgtcaataagtttaagcggtgcagcaccgtcgtaaacagatacatcaaccgaggcaaacgggcgggcgctaaaattggtcgtgaagtctgaaccactcggcggtgcttgtaagatttcctgattcaagccgatgaacgggaaagaaccggtgaccattgcgttaacagcctgttcgatagtgaacccggtaaactcaacgcctctggttacgatatatgaatccgggtttccgcacttacctttcaaccacgtaagaatcgaataggtcttacacaagttaccggtttccagtttatctgcgatacgcaaatcggcttgggcgtctgcttgggcggttagagtgtgctggatgccagcaccggttacaaccgtagcggttaccgcggtaacgaggaaagctttatcgttattaccggacagaccatcgaattgcaccaggtcacctacttcaacaccatcggttacaaagctaccggtagcccgtgtaaaagttttcgctaccgggtctactgtgacgccgattgccgatgcggtggaacccgctacccatgaactagccattgcgcctgctaacagctcgtcttggctttccgcacttagctcaatagcgtactcacctgttacctgacggttgccagtgcggatagctgatgtttcgttgctgccgtctagctcattagagatgagggcgtctcgggttaccgcggggatgccgcctgtgttgcggagtggctgccataccgggttagctggcgtcacccccggggtttcctcacgaacgtaaaattgcgcggtcatcgcgcctttatatggttgtaacgccattatctaatcctcgctgtaaatgctatgaaattaattgagagaggcctcttcgcccatccattctcaacaataagaggaccgaggcttactgattgtacctcagcacagatttcgttacgactaaaacagttaccggccgcgaaggtagcgtttagcttatcagctaatcggttgataggggcactacctagagctgacccaacgttaatgtccacctgataaacacccgaccgttgttcagtccagaacaagtcggcttgctccgtatcggacaacagcatataacttgcaagatacggcatatcagtagacgtcggcgcatcgatgttctctaacgcaaccttgatgtcgttgtccatgccgaacgtcaccagcgccgtgtcgaatgctttcgttaagtcctcaaaatatgttgccattatttcaccttagaggcttcttcatttagtagttgctggaacctgctgacgttagtcctgaccataccttgcggggcctgttgcgaccaaccgtactccagacgttgtgcatacggtaaattgttagtgagggtgaaggtatgccagtccgcagcgtttagcacgaaactcgtaaccttaccggttgctgcgttgcccgatttatcggtagcgtccgtagtcccatcggcgggggtgcttcctgaagccatccagttcatacgaaatcgcccggtatctacgggactcgctttgataatcgcagaaaacaactttatagaaacctgacgcattaccttctcaggattcttcttagccttttccacgaacttggacacatcaagcgcaaaactcattttctcacctgaataaagtatgccacaacatcatcgttaaccatcttcttctcaatagcaactacagaccattgttcaccgctaaactgcaccttgtcatccatcttcggaactacgctgtaatccgctttaaccatcacatcgcctgcctgtatagtagttccatttaccagtccagcgttaacgggtacaggaaccgctttaaggggaagtacaacgtcgggcccccactcgtactcccctaaaacagggtcccatgttttctggcctttgcgcaccaaagttaccgtgctgccgtattttgatagtaggcgagtacctacgccttgcatccgtttactaaaagcggtgctcatcacacaccctccagtcttgaaacgaccagtagtgcggatggggacacaccccaggacgtaaccgttgccacttgcgggtataacccgccgaagttggaaccggaggcgtcgcgcataatctctaccgtaaaagtctgccctgcggtagcattcacaactacacgagattcaatggggattgtggaatccgcgctcgccagtttagtcgcggcgggtgatccgaattgcacaccacctagcaaaacccgcgacagaaggatagacgttccgctcgccccagtacgtccggcttgtaacttaacacggatggcgtagttgcccgcggcgttgaacgtaactacacccgacgcgttaatcattaccgggtcggcgcttgttttctgcgcccctccaaaggctacggttaatgctgtacccgtggtggtcggtgcctgaatatctgtagacgacgcgcgaaggacttctacttccttagcaccgtacagcatagagtccgccatctgagtagtaacttcgcgcaacttttcgggtgtaattagcccggactggttatcagggaagtttgccccaatcagggcgaatatttctgatttagtcttagccatgtttaacccctgaagacattaaaggagtaggcattattgctcccgcataaaagaggacgtagcgcgtcatcggctgccgttatgcttactgtaccgcctgagtagccattcttaaagtaggacacggtcaccgcgccttctacccgctccgtctgcacctcgcgcccgtctgtagaccctcgaacgtccgtacccgcgccgtactcaaccgcggccatcacctgagcttgaataactaacgatggtattacgttggatggctgcgggaacccgtgcaggtcgatacccgttctggggaaagccagtgcctggtttgctgaaacgcgacgcccgcacatctgagactcaaaaagacccacgtataccgcgccattacggagagaggcctccgctgcgatgtcatcttctggtagttcgagaccgtacttagcggccagcgcccgcccgtcttccaggctaacataagagtcggcatttggcaggccttgaccggtttccacgataagcggcataaattattcctctacacttttacggcgacgacgttgcttaggctccccgccaccgttgtaatgctgttcttctgctgtcggctgagtctcaattaatgcgcccgggttcgcttcttcgcgcatcggggttaactgcccatcaacctcaaccacacctttgtattgctcccttactacataattatctgccatgtctaatccttaaagcggcccggtggccgcatgtatttaacttactgtcactacagtactatctgaaatgatattaccgtagccgtcgtgcgctacgactttgtaagtaccggaatccgccaccgcggagttggccttagtgtaagtcaaagcggttgcacccgcgatagcgttattatccttataccattgaaccgttttagggtccattccgtcggcgagttccaccgtaagggtgatagcctgcccagcggttaccgcggtagtatcattgaggccggtggagaaacgcaacggcatcacgttctccatatcaatttcaacctgcccgtcctgcggactatcatcagatacaccaacaatacgacgtttaattacatcaaccatttttatcctcctcaggatacagtaccagcgtttttaagggcggttaacagattagtaacggcggtgcgcaatgaagtcacatccgtacgcaacttattgtagttggtgaccagtgcgtcgaactcttctttcgtaggtgcagcggcggcggctgcaccgctacccgcgctaatcgccggtggtgccgctaccgtagcggactttttaacccctccgattgctgttgtggtcgccgcgggaatatcgaattccgcgttaacaaggctgcgtggtagccctttacctgtctttgacatcatgtagtctccatataaatgaaaagagggaccgaagtccctcaagagtataccctacgattaagcaccgacgccagttacaaggaacgcaatcggcacatgcttacggtcaactacacgattccagttggtagcgttagccaggtcctgccagctagcggaacgtgcgatagtctcggtaccgttgccggtgattacagcgctggtgaagctgtaaccgaacggatgcagcaaccacgttttacgcgtccacagggtttcaacaccaccaccgttaccgcgcgactcttcgcgctcatatgccagcggggtttccggggaaccttcaccgtaaccgatagcaccacgtccaaagatgatggagatgaacttgcgctgtgcaccctggcctactacagtcatgctgtcatcaacgatgacgcgatagccctggtaggtagcgaacatggtattgttctcagcatcacgaatgaaatcgataagctgagctttacgggcctgtgcatatacgaagctgtgcatagcgatagcaccgagaacctcgccaccgttacccatcaacgcatcgcccatagtctgagtagcgtcgatgaatgcgcctgcgtcgaagccaagggtggcggagacgtctaccaccatgtcgttctgctcgtggtattcgtcggtagcggctacgttgtcgttgtacaagccgagggcggtagcaatcagacggcgctgtgcttgacgttgccagaagttatccagacgggacgctaccgactgcaacgggttctggctggtcaattcgacggtcaggtcagcctgaccgaagccttcgttcaggtaggcgacgcgggccatcatctcaccggtctgaatagcgcgcggagtagcgatatcctggtacacatcgttcgaatagttaggttcgatagaggtgtcgatagctctccagaacggcaagttagcgatgttggatggaccattagcaatctcggccgcgtacggggttggagtaaggataccggagtcgaaaaacgcggttttctctaccgggtcttcggtcatgtaagacgccagcacagggatattgccagttacgatgttgccaatagtggtaattgccattatttatttcctcagggctttaagttgccgttcaaattcggcagggttagatttatagagcgctaaacgctcactttcactcatgtctttaaacgctggtgcggccccgccgcctttattcccggaagccccgccaccggaagctgcatttgctttaatcaaatgtgaaaaagctttatgttcgcgcaggtatttgcggaattgctcagggtcggtagtaacaacattgccatccgcacccatgaatttagtcactacgtcttcgccttcaaactcggtcttaacgaacggcgccaggatttctaccgcttctggggtgataaaatcaccagcgaaagaacctaaaacagccttacgttcactaccaagaattcgcgcggctaaagaagcatagcgttcttctttctctttcagtacgggttcatactgactacgaatcgtcttttcaaattcgtccattttaccggcagccttcagtgcttcctggtgggcgcgttgccgttcttcttcggcttctttagcccggcgcgcagcttcctttttctcgctaagtaacgcttcctggttagccttaagcccggccacttctttctcaatcatcgcctgcacttcttcggccgtgtacatttttggtgcgtcaccgccgcccgcgggtttaaggtcatctggtgcttcgtcttgaagcgggtaacgtaaaaaacgattcatagtcaatatgtcccctggactttaggacaccgggcctcccggtgtttctgtaacaagaataaattattccacttactaaggcaactattccagaatattccttatgtactcctgcaaccggaatactttcaggcgcaattgccgtgtgcactcggcgttctgtacatcgatggccaggtcttcgtcggcatcactacttggcaaggccattttgcacggcggctgcattagtgtcatatccggggatggaattagcattctcggcggcgcgccgtttgattgacacccgctcaggagggaacacgcaaacaacacgaccaggacgggtaacgtatttgataacttcacgggtgatagcctccgcgttttctttaccttcggattcggcggcggcggccttggcctcattctgctgctgccgttgtgttttcttcgctagctcggcctgtgctttctgctgttgctgtgaaacaagattggcgcggccttctacccaaccgcttctgtagttgtactggccgtataaccacaaggagaggataccggctgccgtgactaccgcggttactttccaattcatagtatcccctttctttatacgctaagcccggcttacgccgggcgttaaactcgtatatctgtaaaccttatacggcgaatcttagctaaatactgaaagcagtatagctcggcccttccgaatgcgttcgcggttacgctagggcgttgtgacgcccgcttcacgcgacccgcaccgcggtacgctgaaagcgtagttagcgtagcatgtgaaataatataagtctactttttgaataaagtattccttaatttacattattcatgaataactggaataatcgcggataaaattaaggccccttcaaggggcctttcgttacttaggttccgagaccttagccgctttgctaaccaccgacacaccgtcggcatctactgccttgacgaaatattctccggggtctttaacggttagttccccttcaacgtaagggacgttgattacctctttaccgtctttaaaccattgcaaatcgtaaggcgctttgccgcctttcacaactacggttagcttagcggaaccgtctttcaattctacatctgctggctgtgtatcgaagtacacatcacccgctccgtcccgatacggcacttcgtacaggataccgtcggtgattgctaccagtccggtcttatcagcaaacggcatttcatctaccgattcaccgagaaccgattcatctttaatgtacacgacaccttcgcccacaccggacacacgggagtactgaaccacacgtcgcgaaggcacatctttaactttgaaaaagcccatctctattcctctttcagataatcggcaacacgcttatcgagttccgccatctctttaagagttaacggacgcccgaatccatctaccgatattacacgaaattcttccggggttatcccactattacgaaaaatcttacccctgacagggccgagggcttcgtcctggaaccaggccggctgttgtttaagaaactcgtaataggtggtgtccgcacttacctgagtgccgccgtccgcacccctggccgcccgttttgcgcctttatcaaggaagtcgaattcggaactgattacaggtgccgtggtacttcggcagttaggatgcgccgggggcgtcggccccttaccgatttcccacgtcattccgtccctggccctgcaaatcgtactggtacggctgtccagcgttgacacccattcgtacttctcgatgatgtcaccgttctgccggtacgtctcgttgcgggcttcgttggatacgtgggacagtgcggtgcggattactgtagctgcgttacgttctgaaatgtccgccagcccaccagcaccaacaacattcttaacaatctgccgtgtggtttgaccttgtacgaaacccatcttaacgccagttaccaggcgcgcgacttctgttcccccccagcccgacatcagtttagtgaaatcgacaggtttgtcgcttagtgacagcggctgaaactcggcagcggcccacacctgttcggcgccaggcgtaacaaattccgcgtttacgttggcgaccagtgcctttacattccactcggcctcatacgccgccagttccttaaggtcttctgtcagctttgtttgccagtcatcagtaagcccggttagcgctttttcaaggtcgcgtaacagtttattaagccgcgcggtgcttcgcctgtcgtcaccgaatagcagcacctgccgtttgatttcgccccgcatctcttgaatgaacggcgcgaggtctttcacttcgccggatgcggtacgttgtaaccatatctgatggctgataatcgattctaaaagactcatgtacccctcctgtctctacgcaacaatcatatcagccagtagggtatagacataatcatgcgcatttaaggttaaaaagagcattgacaaatggtatatctatatataccatttaatcatacgcatttaaggttaaaaagagctcggttaaattctacatttgaatgtagaatttaaacgtacacttaaacaggagtgatatacatgaaattgcaaacattagagttcccgtatggagaaatcactttcgaccgtgacaactttattaacgtaaacgagttggtcgaccagatgaaccgttggcgcttagacaatggtatgggctttaagcacctacggcagctaatgacgacagatgggtttaaagatttcaaggctgagtgtgaaaagcagcttggcattaaggatgccgtcaggattacacgagggcgaaacggctccacttttgtgtgccttcaccttgcggtatatattgcggagcaatacagcacttatttccatttcctcgttatcgaccgttttattacccaacggcaggtcgaactacgaaacattggcgcggttagctttgtcgagcttaacgccgccgttagtcgaatggttgagcgcacggagggaagaatcggccacagtggacacttcatacaggtggctaaagcggtaaaggaatccgtagatatccgcgaagtaacgggtttcgatacctgggactctcaggatgcgaagaccaatcagttgcgaagtgacatccagaaagccctggtgacgcttcttgacatggaggccattaatacatggggcggactgaaagagacgatacctcgggttgtgcgtaaatgtgcggctaatatacgttaaaactaaggccccataacggggcctttctgtttactgctcctgttgttgcaccgattgagggatttccccctgaacttcagtagcgaccggtaatggctggtcggctaccgcatccttaatatcagcgtctgtccagtcggtgacaccggccctacgcaacgcggcatagtaggctgtaacaggcagaaatcccgcattaatatccgccatccatgcagccctgtcttgagctgtcataggttccaggaagaagtccatattaaggtggaattcaacctcggtatcttcgggtttacccaacattaccgccacccaacgtaaagcatctgtatatgcctggcttacgttacgcgcaattgtcgccatgacggatgtatcggcaccgcgctggattcgggcagattgtgcggtgatttgctgagtcggggtgataagctgcgcaccaatctggatagcctgctgctctttgtccagcatattctggcgggccaggctattttcgccggcctggataagctgtgcgctacctccgtaccccaggttatgcccacgccgactgccaaatttgataccgttggggttcgcttctttgaacgactgcggtgttaagttctctcctgggtagataaacagcgtgggttgccctacaacgaagctggactcctcattgtccgcgctgttgcggaaatgcccgatattaagctccgccagcggcaacaaaggcgcgtcgtcgatagtggcgtcgttattggtcgccccaataaaggtaaacggaattaccccccgtaatgattcccctaagtccgggtaaatctccacgacgtcttcctgcgcaccaccttcggcatcgaaacgaaaaagacgttgacggtagttgccgtcgctatcgatgtccaggacccggtactgttccccatattttgtttcgaactcgtttcccggttcgtggtactcccatgtctcgcgcagcacgaccatagttacccggtttacggaaccgacgcgcgtgagtcgccagttaacgatgttctcagtcgtgtaaaaagcgattgtaggattaagcagacctgcgttctgttcagccgccgtagcagcgccggtttccggagcgtcgacgagaagaccaccacgacctaccgagtcaatctccatgagtgtatcttgtgcgtgctgtatcaggcctacaccagacccatccgcatttttaagcaggtattccagctccttcgggatattgatttccggctctttgcgcatgacgctaccgaccatccccgacagcgtgcgccgggtgaagttgtatacgatgccgccagcctcatattctgcctggcgcgcttccccgtacgctttatctggttcgttcagtcctacgttgcgtaagtagcccaccagttcacctgcaagcgcgtgacgcaccttctgccacttaggggagtaatgcaaccattcgcggtgcttggtttttacgccagaaccctggccgtttgctgttaacatttaataatcctcttaaagtgcgaaagtaaccgggatatgggatacaggtttaaccaacggcatttcgtaggcgatagggtatcctgtcgcgtcgttctggtggtcgttgccgcttgtcttatccggtataccgtttttatcgtaggcctgttgctccaggcaacgtgctgtaaccgggcaagcctgttcgttgaccattaatctaccagactccagcgctttattcaccgatgctacacggtctttaacggcagggttaaccgattttgcacgtatctcgaaaccagcgttatgcagttgtgcaatatccgaggtacttgcatcagtcgatttacggttcttaccactggcgtccggatacataacaatgtgatgcccgttacgcttccatcgttcggtaatctccctgaccacatccggggtgtcgaacatatccaccagctcggctaccgcgtgccagacatactcacgctgaacgtatacggtgctagccatatgcccgacgttgaagtcctgaccgatatacagcgtttccccaggttgtatagtctctcgactgctgttcttgcgtcggtcgtaagcataatacacgctgccggatgtcaggttgacaaattcaccatcgatgtaggcatcaattaactggcctggatacgtatcccgcaacgactgcacataatcttccggtagaaaggggttggatgttgtcgaggcctgaatcatttcatagcctggtttcttttttacagcccatctgtcgtgcacaaaacggaagccttctggcgtcgtaaataccgaaactgtattagcgggcttcggggtaatcggacgatatgtacgcggcaactgacggttacgggcgataactttgttccaggcgtgctcggcgtggtctttatttaacgtatccaattcgtctatttttgcgcgaaacgattcgtagccaacaattcgcgccgggttatccaatgtacgcaggacaaaatccccgaattgcccggatgatgtatagattatgttgtctgatttattgtacttgtaacgaataccccaatcagacaacttctcttccatacgcggagcgaggataaggcgcaccaggtcgtatgtcggttcatacatggcgataagtgaatcactaccaccttccatgctgtcgagtagggcggagttgcacatgacctccgatttacctgtgccgaatccagcgacgaaggccgggaatttacaatgcagattaaggaacgcgccttgcggttccgtagccgttatatcaacgttcaccggacaccaccttaatagttacttcgctaattggttcgtcgcgcgcttcttccacaacagttttattaagcccaagttttgctgcggcgaacgtagcagatatcccggcagcaccggtctcagtgaaatacgcctcttccaaggcctgcgccgtttcgtatgcttcagcgaaggcaggaacctctcgcagccacagcttgataatcggaacggttacgcctatgtgtagtgcgaaacgggccagcgacggcggtttgtcctggataagcgggcgttcgtcgcctttagacgtgggtacgagttcccatgaggtacggtcgaagaaccggattaactcgtcgcaatagttcgggtcccatagttcggcggaatgccgggatgactggtacagactttgctttacgcgcgggcgtttacggcgacggtttgcactaaccgcttcttcgtgcgcggcggccactacttcggcgtccggctgttttaatttgagtttcatgcaatcctctcttaaatgcgtatgaccatcatagcgcatcagacgccccctgagcgtctattaaggattgtacaggggaatgaaggcctgtgtacagaaaagcccgcagaagcgggcttggtggtaggctattttacagttctacagatgataaatggttggccgcggacttaaccgcgtctttaaggctatcgtacgcggaagtaaaagacgtatgtactcctttcgagacaactatagcgccgccgttaaagtacgcttttgctctccagccaccatttaccttatcaataaggatgtagtgactaccttcttccggtaccgtaaccgtgttttcgtctattacttcaaatttagtgcccatgttcgccctctcctctttagcaatgctcgtctttataaacccaccatttacctacggcttcccatcgccacccgcgactatggatggcatagaatctacggccaaatactgtttccccgtaccacatgacaaatagcttcaccgcggtagccgcaccaattttatgagcattcaaacggcgatacgctagtgacatcctcgtaagacttaccgcctcaacaaatggccaaagaaacagaatccacatcaggtatataacaacggcaagcagtaatgttgtgaatcctatacccatgaggatgagaaaactatccatcacccctccagcttcatacggtcgttgcgcgatagtccacgtttcgtgtagatgaccggtgacgttttcgtatgggtcatcagtcggttgttatagataacatgttgctcgcattttgtgtcatggcggtacttggataatgtcatctcattaagtcccgtctcccggcacgtatctgctaatgtgccgtgtgtctcgattagcttggggatactggtaatcattctttatcgcaatccttcggtttattcaggtcgtaacggttaataagctcccggcgatgctcgttcaggtagtctaatgcggcctggtgcgccttaatttgcgcgtctaccgcttcaatcttaaacctgccgttggcggcttctaccatcatcttttcatattcacggtcggtcataaaactcatcctcggcaatggtttacgtctcattctttacgcgcccccacatacggttgagatatttgttctggtcaggccctgggaaactattgcgttttaacagttcctcgcgtgtcgggaatggcgtatttcctaccttaccgcccaccttaaacgttactgctacttgcccgttgtcgctcattcatgcacctcccatccgcatacgatatatactttcatttttgccccctgcataagctgctttcagtgtctgcatcgcggcgaaccagaacgcgtcagcaggaattcgtgtgctcagattattaacggcaatgcgtgccatcaattgcgcgtctttgaatgcttcggtgtcaagcaccggtttgcaatggtaaaaaactttatcgctttgtaagttcatcttctctactccgttctcgctgtcgatgaactaaatataatagggttaatgctaccagtcaactattatttcgggatacagataaaaaaaaatcccggtaagggtcagttaccgggataaaggagctagagggatgaataagcaggagcgacaccagtatcttaggaatgctcatatgtgtcaacctactttgcttcgcggccgatggcttccgcctgttgccacagcataccgtcgaatagcgcaacacgaccggcggcgcgacgacgtaatccgaggagtgatttgccgttctgataatggaactgagttaacttattccgcagtggggcaacgtcgcccttacgcagggcctgccctgttccggtagaagccgcaatcacaccggcgccagcgttatacaccaggtcgcacatcgcatcgaactgtgactgattgagcgtcggatgcgcaacggcgtccaccgcagctacggccttagccatatccttatgcagcaacagaagaccctggccctcggtaatcttctggccttctttcacatcagcaccgtagtgcccataaccaatagtaaggtacttctcgttcttcgttgctttgtacgcggttccacggaaaccctcgaacgcggcggtgaagtgtagcccgttatcactgatgtttcggtttgccatacatcctccggtatttgtagtggcgagcgcctttcacgcaaatgtggcgtaccgatttaagcgcgaatatggcaacaagagtcgtaattatgaatggcgggatgtcgccggtaaagagatgagcaagaccaccagcggcggtcactacggacgccatatacaacagacgaccgaaaagaccgtcttcaatccagtgagcgtagatattaattaacgaggtgatgatgattgcgaccagacaaatgacaccaattggtgagtcgagcatgttatttcccccacggtaacttaaagttgctgagggacgagtcggcggcctcaaataacttaagccagaacatacctacgccaaaaggaaccaggtactgaccctcagattgcgataactcatagtaacttataattatcggggatgcgtacacagcgcaacaggccgaagcggtaagatggcacaatcgcaaccatggcgaaatagcctcacgcttcttaatctgagacacggcacccccggcgaggcccgccacactaagccaaaggtatttatcgtccacaataaaaaatctccgaaagttggtttaccttcggagattaacatttatacgacaattttagtaacagataattcggatttatcctagtcgatgtgttatcactcccccatcagtattagggtcgaagcgcttcgctatctcgacagcagcagtagcggcggcacccataaacatcgccgtaagtgcatacggagcaccggagccaacggccgctgcgttacccgcaattggtataactgagcttaacggcgaatcccatgacttgaagaattctaccctgaaaatacggttatcctctttaacaaacaccagcgctgaaaagtcgaagtcgtagtacttaggcgtcaatatctcggcatcgacaaccaacagcctacccaccccggcgtccccggatacgcccataacgaagtggtcgttctcgtaaatcttggtatccgtatagtatttaaaattccccgtgacacatgtatcgcaggccatcgtttcaccgtcaaaagctatagtagtcatataagttacctgaacgtataaagtcgaatagatggcttgtgcgggatatcggaatacccacgtttacaccacaacttatacagtttaacagccatatgcgcccgggcctggctactgaactcaccgaggtttaccactttcttgccgtcgtaagagtgcgcacgccacagacctttgcctacgcaatacttcggctgatgcacacctggtatacg

(19) United States
(12) Patent Application Publication
(10) Pub. No.: US 2026/0207687 A1
(43) Pub. Date: Jul. 23, 2026

(54) BACTERIOPHAGES AND USE THEREOF FOR THE TREATMENT OR PREVENTION OF INFECTION CAUSED BY SALMONELLA GALLINARUM

(71) Applicants: Shafiq UR REHMAN, Lahore (PK); Zafar HAYAT, Sargodha (PK); Purdue Research Foundation, West Lafayette, IN (US)

(72) Inventors: Paul D. EBNER, Lafayette, IN (US); Saud UR REHMAN, West Lafayette, IN (US); Shafiq UR REHMAN, Lahore (PK); Zafar HAYAT, Sargodha (PK)

(21) Appl. No.: 19/139,801

(22) PCT Filed: Dec. 20, 2023
(86) PCT No.: PCT/US2023/085171

§ 371 Date: Jun. 16, 2025

Related U.S. Application Data
(60) Provisional application No. 63/434,826, filed on Dec. 22, 2022.

(51) Int. Cl. A61K 35/76; A61K 9/00; A61K 9/48; A61P 31/04

(52) U.S. Cl. CPC A61K 35/76; A61K 9/0053; A61K 9/4816; A61P 31/04

(57) ABSTRACT

A composition of bacteriophages comprising at least one phage selected from TTS1, TTS2, TTS3, TTS4, TTS5, and TTS6, wherein the phages are optionally encapsulated; and a method for preventing or treating an infection caused by Salmonella Gallinarum by administering to a subject a therapeutically effective amount of the composition.

### CROSS REFERENCE TO RELATED APPLICATIONS

This application claims priority to U.S. provisional patent application No. 63/434,826, which was filed Dec. 22, 2022, and which is hereby incorporated by reference in its entirety.

### TECHNICAL FIELD

The present disclosure relates to a bacteriophage-based treatment for infections caused by Salmonella enterica serovar Gallinarum (Salmonella Gallinarum). Particularly, the disclosure relates to compositions of bacteriophages and their use for preventing or treating an infectious disease, such as a disease caused by S. Gallinarum, in poultry.

### SEQUENCE LISTING

A computer-readable form (CRF) of the Sequence Listing is submitted with this application. The sequence listing is entitled 69835-02_SEQ_LISTING.xml, was generated on Dec. 8, 2023 and is 255 KB in size. The entire content of the sequence listing is incorporated herein by reference in its entirety.

### BACKGROUND

Salmonella is among the most common gram-negative bacteria with 2600 serotypes and serovars based on O-antigen (lipopolysaccharides) and H-antigen (flagella) types. The bacterium is usually found in the intestines of warm-blooded animals, including most animals raised for food. Several Salmonella serotypes are considered “host-adapted,” causing significant disease in a particular species. Salmonella enterica serovar Gallinarum (Salmonella Gallinarum) is one such example.

Salmonella Gallinarum infections can cause fowl typhoid in poultry. The infections are usually treated and controlled by antibiotics. The drugs are often administered through feeds or water at times when the birds are at greater risk of contracting Salmonella Gallinarum infections. The use of antibiotics can reduce infections, but it also causes the development of antibiotic resistance. Antibiotic resistance has become a global challenge and results in decreased efficacy of the drugs in both human and veterinary medicine.

The increase in antibiotic resistance has decreased the ability to control and treat bacterial infections. The United States Centers for Disease Control and Prevention (US CDC) state that approximately 2.8 million bacterial infections are caused by antimicrobial resistant pathogens each year in the US (CDC, 2019), resulting in $20 billion in extra healthcare costs (CDC, 2019). The continued emergence of antibiotic resistance and its threat to public health have led to the development of alternative treatments such as probiotics, symbiotics, nutraceuticals, phytogenics, metals, prebiotics, enzymes, and antibacterial peptides (AMPs) to control bacterial transmission without increasing antibiotic resistance. However, each of these alternatives has benefits and limits.

Therefore, there is a need to develop an alternative treatment to antibiotics to prevent and control serious bacterial infections in animals raised for food, such as poultry. It is an object of the present disclosure to provide such alternative treatment. This and other objects and advantages, as well as inventive features, will be apparent from the detailed description.

### SUMMARY

Six wild-types Salmonella Gallinarum phages were isolated from wastewater samples. The phages were characterized and named as vB_SalM-TTS1 (TTS1), vB_SalP-TTS2 (TTS2), vB_SalM-TTS3 (TTS3), vB_SalM-TTS4 (TTS4), vB_SalM-TTS5 (TTS5), and vB_SalM-TTS6 (TTS6).

Provided is a bacteriophage composition for preventing or treating infection caused by Salmonella Gallinarum comprising an effective amount of at least one phage selected from TTS1, TTS2, TTS3, TTS4, TTS5, and TTS6, wherein at least one phage is optionally encapsulated.

In some embodiments, the bacteriophage composition comprises phages TTS1, TTS2, TTS3, TTS4, TTS5, and TTS6.

Provided is a bacteriophage composition for preventing or treating an infection caused by Salmonella Gallinarum comprising an effective amount of at least one isolated phage selected from vB_SalM TTS1 (TTS1) having a genome comprising a nucleotide sequence of SEQ ID NO: 1, vB_SalP TTS2 (TTS2) having a genome comprising a nucleotide sequence of SEQ ID NO: 2, vB_SalM TTS3 (TTS3) having a genome comprising a nucleotide sequence of SEQ ID NO: 3, vB_SalM TTS4 (TTS4) having a genome comprising a nucleotide sequence of SEQ ID NO: 4, vB_SalM TTS5 (TTS5) having a genome comprising a nucleotide sequence of SEQ ID NO: 5, and vB_SalMTTS6 (TTS6) having a genome comprising a nucleotide sequence of SEQ ID NO: 6, wherein at least one phage is optionally encapsulated.

In some embodiments, the bacteriophage composition comprises phages TTS1, TTS2, TTS3, TTS4, TTS5, and TTS6 comprising the nucleotide sequence of SEQ ID Nos: 1 to 6, respectively.

The phages can be encapsulated using synthetic or natural polymers. In exemplary embodiments, the encapsulated phages are encapsulated with natural polymers. Examples of natural polymers include, but are not limited to, sodium alginate, chitosan, gum, gelatin, collagen, or a combination of two or more thereof. Suitable natural polymer can be sodium alginate.

In some embodiments, the composition comprises encapsulated and unencapsulated phages in a ratio of about 1:1.

A method for preventing or treating an infection caused by Salmonella Gallinarum is provided. The method comprises administering to a subject a prophylactically or therapeutically effective amount of a bacteriophage composition comprising at least one phage selected from vB_SalM TTS1 (TTS1), vB_SalP TTS2 (TTS2), vB_SalM TTS3 (TTS3), vB_SalM TTS4 (TTS4), vB_SalM TTS5 (TTS5), and vB_SalM TTS6 (TTS6), wherein at least one phage is optionally encapsulated. In exemplary embodiments, the subject is poultry. In some embodiments, the bacteriophage composition comprises phages TTS1, TTS2, TTS3, TTS4, TTS5, and TTS6.

The prophylactically or therapeutically effective amount of the bacteriophage composition can be administered orally, topically, or subcutaneously to the subject. The composition can be administered in the form of a suspension, feed, water, a spray, or an injection over a period of time ranging from about 1 day to about 60 days. The prophylactically or therapeutically effective amount is from about 10 PFU to about 10¹³ PFU of each phage per ml of composition, in volumes between 0.2 mL to 2 mL per bird per treatment.

Further provided is a method for preventing or treating an infection caused by Salmonella Gallinarum comprising administering to a subject a prophylactically or therapeutically effective amount of a bacteriophage composition described herein above.

### BRIEF DESCRIPTION OF THE DRAWINGS

The present disclosure will be more readily understood from the detailed description of embodiments presented below considered in conjunction with the attached drawings of which:

FIG. 1: shows transmission electron microscope (TEM) images (a)-(f) of Salmonella Gallinarum phages vB_SalM-TTS1 Myovirus, vB_SalP-TTS2 Podovirus, vB_SalM-TTS3 Myovirus, vB_SalM-TTS6 Myovirus, vB_SalM-TTS4 Myovirus, and vB_SalM-TTS5 Myovirus, respectively, used for phage treatments in the first and second animal trials of chickens.

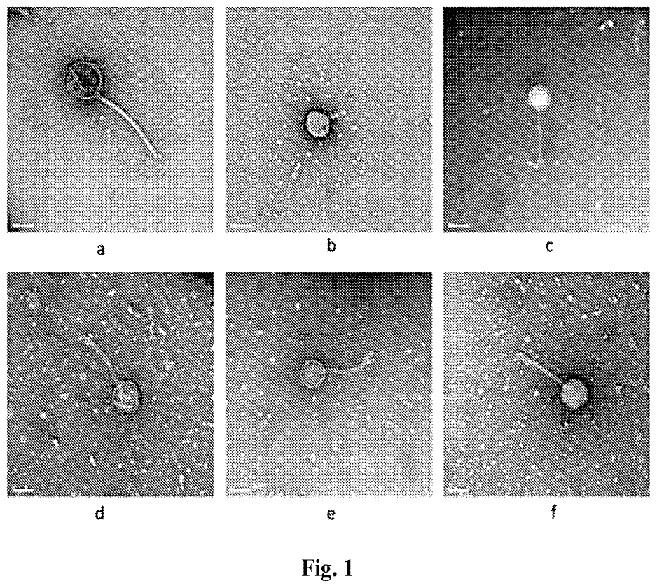

FIG. 2: shows Salmonella Gallinarum concentrations (OD600) over time in cultures treated with Salmonella Gallinarum phages (six total phages) vs untreated culture. *═P<0.05; comparison is within timepoint.

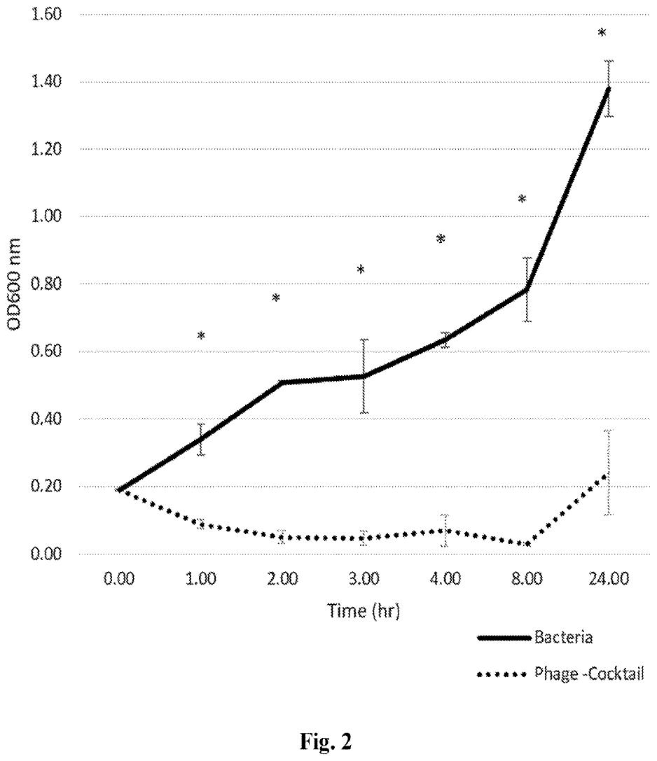

FIG. 3: shows Salmonella Gallinarum phage concentrations (log PFU/mL) released from capsules over time when exposed to simulated gastric fluid (SGF) for 30 min followed by exposure to simulated intestinal fluid (SIF) for 120 min. 0 min indicates the time when phage capsules were added to SIF.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the claimed invention is thereby intended by the description of these embodiments. On the contrary, this disclosure is intended to cover alternatives, modifications, and equivalents as may be included within the spirit and scope of this application. As previously noted, while this technology may be illustrated and described in one or more preferred embodiments, the compositions, and methods hereof may vary.

The terms “bacteriophage,” “phage,” “bacteriophages,” and “phages” are used to refer to a functional phage particle or particles, respectively, comprising a nucleic acid genome packaged in a proteinaceous envelope or capsid. The term also refers to portions of the bacteriophage, including, e.g., a head portion or an assembly of phage components, which provide substantially the same functional activity.

The term “lytic activity” designates the property of a bacteriophage to cause the lysis of a bacterial cell.

The term “PFU” means “plaque forming unit,” the number of virus particles capable of forming plaques per unit volume, as it is well defined in the art. Lytic bacteriophages lyse the host cell, causing a zone of clearing (or plaque) on a culture plate. Theoretically, each plaque is formed by one phage, and the number of plaques multiplied by the dilution factor is equal to the total number of phages in a given test preparation.

The bacteriophages were isolated from wastewater samples obtained from wastewater treatment facilities in central Indiana (US) or wastewater and poultry samples from the peri-Lahore (Pakistan) area. Identification of these isolated bacteriophages in the examples showed that the phages were vB_SalM TTS1 (TTS1), vB_SalP TTS2 (TTS2), vB_SalM TTS3 (TTS3), vB_SalM TTS4 (TTS4), vB_SalM TTS5 (TTS5), and vB_SalM TTS6 (TTS6).

The term “composition of bacteriophages” refers to a composition of bacteriophage, which can be the same or different. A composition comprises at least one of vB_SalM-TTS1 (TTS1), vB_SalP-TTS2 (TTS2), vB_SalM-TTS3 (TTS3), vB_SalM-TTS4 (TTS4), vB_SalM-TTS5 (TTS5), and vB_SalM-TTS6 (TTS6). The composition can comprise two, three, four, five or all six phage, such as TTS1 and TTS2, TTS1 and TTS3, TTS1 and TTS4, TTS1 and TTS5, TTS1 and TTS6, TTS2 and TTS3, TTS2 and TTS4, TTS2 and TTS6, TTS3 and TTS4, TTS3 and TTS5, or TTS3 and TTS6. Other examples of combinations include, but are not limited to, TTS1, TTS2, and TTS3; TTS2, TTS3, and TTS4; TTS3, TTS4, and TTS5; or TTS4, TTS5, and TTS6. Yet other examples of combinations include, but are not limited to, TTS1, TTS2, TTS3, and TTS4; TTS2, TTS3, TTS4, and TTS5; TTS3, TTS4, TTS5, and TTS6. Still yet other examples of combinations include, but are not limited to, TTS1, TTS2, TTS3, TTS4, and TTS5; TTS2, TTS3, TTS4, TTS5, and TTS6, and TTS1, TTS2, TTS3, TTS4, TTS5, and TTS6. All permutations of combinations are contemplated and encompassed. The bacteriophages in a cocktail/composition can be, and preferably are, formulated together, i.e., in a same vessel or packaging, although they can be prepared as parts of kits in which the (or some of the) bacteriophages are formulated or packaged separately and combined when used or administered.

The term “poultry” means any domesticated bird used for food. Varieties include chicken, turkey, geese, ducks, Rock Cornish hens, and game birds such as pheasant, squab, and guinea fowl. Also included are large birds such as ostriches, emus, and rheas (ratites).

The term treating is intended to encompass prophylactic treatment as well as therapeutic treatment.

The phages are natural predators of bacteria and have unique properties such as host-specificity, ability to lyse bacteria, self-reproducibility, ease of isolation and propagation, ubiquitous presence, long shelf life, and potential for affordable scale-up. The phages are host-specific and can be susceptible to various environmental conditions, including temperature and pH. Each of these factors is important in considering bacteriophage-based treatment, as each factor can reduce phage viability and activity. This is true for the production, storage, and application of phages, e.g., through feed or water, an oral treatment, or an injection, such as an intravenous injection.

Provided is a bacteriophage composition for preventing or treating an infection caused by Salmonella Gallinarum comprising an effective amount of at least one phage selected from TTS1, TTS2, TTS3, TTS4, TTS5, and TTS6, wherein at least one phage is optionally encapsulated. Some or all of one type of phage can be encapsulated or, when two or more types of phages are combined, some or all of one type of phage can be encapsulated and none of the other type can be encapsulated (i.e., unencapsulated), some or all of every type of phage can be encapsulated, or all of one type can be encapsulated and none of the other type can be encapsulated (i.e., unencapsulated). Any and all permutations of encapsulation/un-encapsulation are contemplated and encompassed.

Provided are encapsulated phages suitable for oral delivery for preventing and treating the infections caused by Salmonella Gallinarum in a subject, such as poultry. The phages can be protected by encapsulating them with natural or synthetic polymers. The phages are encapsulated to protect them from gastric fluid (GF) and therefore are suitable for oral delivery. These polymers protect phages from harsh acidic conditions, potentially leading to phage inactivation or loss of phage titer. These polymeric encapsulation materials also protect encapsulated phages from digestive enzymes and bile juices. Encapsulation allows the controlled release of phages.

The suitable polymers can be natural polymers. Examples of natural polymers that can be used include, but are not limited to, sodium alginate, chitosan, gums, gelatin, collagen, or a combination of two or more thereof. The suitable natural polymer can be sodium alginate.

The phages can be microencapsulated using sodium alginate and CaCl₂). The encapsulation efficiencies of all six phages can be between about 93-99%.

In certain embodiments, the bacteriophage composition can comprise a combination of two or more phages selected from TTS1, TTS2, TTS3, TTS4, TTS5, and TTS6, wherein phages are optionally encapsulated. In certain embodiments, the bacteriophage composition can comprise an effective amount of phages TTS1, TTS2, TTS3, TTS4, TTS5, and TTS6, wherein phages are unencapsulated. In certain embodiments, the bacteriophage composition can comprise an effective amount of phages TTS1, TTS2, TTS3, TTS4, TTS5, and TTS6, wherein the phages are encapsulated.

Provided is a bacteriophage composition for preventing or treating an infection caused by Salmonella Gallinarum comprising an effective amount of at least one isolated phage selected from vB_SalM TTS1 (TTS1) having a genome comprising a nucleotide sequence of SEQ ID NO: 1, vB_SalP TTS2 (TTS2) having a genome comprising a nucleotide sequence of SEQ ID NO: 2, vB_SalM TTS3 (TTS3) having a genome comprising a nucleotide sequence of SEQ ID NO: 3, vB_SalM TTS4 (TTS4) having a genome comprising a nucleotide sequence of SEQ ID NO: 4, vB_SalM TTS5 (TTS5) having a genome comprising a nucleotide sequence of SEQ ID NO. 5; and vB_SalMTTS6 (TTS6) having a genome comprising a nucleotide sequence of SEQ ID NO. 6: wherein at least one phage is optionally encapsulated.

In some embodiments, the bacteriophage composition comprises phages TTS1, TTS2, TTS3, TTS4, TTS5, and TTS6 comprising the nucleotide sequence of SEQ ID Nos.: 1 to 6, respectively.

In some embodiments, TTS1 having a genome comprising a nucleotide sequence of SEQ ID NO: 1. In some embodiments, TTS2 having a genome comprising a nucleotide sequence of SEQ ID NO: 2. In some embodiments, TTS3 having a genome comprising a nucleotide sequence of SEQ ID NO: 3. In some embodiments, TTS4 having a genome comprising a nucleotide sequence of SEQ ID NO: 4. In some embodiments, TTS5 having a genome comprising a nucleotide sequence of SEQ ID NO: 5. In some embodiments, TTS6 having a genome comprising a nucleotide sequence of SEQ ID NO: 6.

In some embodiments, TTS1 comprises a genome comprising a sequence as set forth in SEQ ID NO: 1 or having at least 95% identity, more preferably 96%, 97%, 98% or 99% identity to SEQ ID NO: 1. In some embodiments, TTS2 comprises a genome comprising a sequence as set forth in SEQ ID NO: 2 or having at least 95% identity, more preferably 96%, 97%, 98% or 99% identity to SEQ ID NO: 2. In some embodiments, TTS3 comprises a genome comprising a sequence as set forth in SEQ ID NO: 3 or having at least 95% identity, more preferably 96%, 97%, 98% or 99% identity to SEQ ID NO: 3. In some embodiments, TTS4 comprises a genome comprising a sequence as set forth in SEQ ID NO: 4 or having at least 95% identity, more preferably 96%, 97%, 98% or 99% identity to SEQ ID NO: 4. In some embodiments, TTS5 comprises a genome comprising a sequence as set forth in SEQ ID NO: 5 or having at least 95% identity, more preferably 96%, 97%, 98% or 99% identity to SEQ ID NO: 5. In some embodiments, TTS6 comprises a genome comprising a sequence as set forth in SEQ ID NO: 6 or having at least 95% identity, more preferably 96%, 97%, 98% or 99% identity to SEQ ID NO: 6.

In certain embodiments, the encapsulated and unencapsulated phages are in a ratio of about 1:1, in which case the encapsulated phage can be of a single type of phage or a combination of phage, in which case less than all of a given type of phage can be encapsulated or all of a given type of phage can be encapsulated.

Salmonella Gallinarum mainly colonizes in the gut and can also colonize some pre-stomach parts of the digestive tract. The bacteriophage composition comprising a combination of encapsulated and unencapsulated phages acts in such a way that the unencapsulated phages can be immediately active pre-stomach, and the encapsulated phages can be protected through the stomach to reach the intestinal sites of infection.

Method for preventing or treating an infection caused by Salmonella Gallinarum is provided. The method can comprise administering, to a subject, a prophylactically or therapeutically effective amount of a bacteriophage composition comprising at least one phage selected from vB_SalM TTS1 (TTS1), vB_SalP TTS2 (TTS2), vB_SalM TTS3 (TTS3), vB_SalM TTS4 (TTS4), vB_SalM TTS5 (TTS5), and vB_SalM TTS6 (TTS6), wherein at least one phage is optionally encapsulated.

In certain embodiments, the composition can comprise the phages TTS1, TTS2, TTS3, TTS4, TTS5, and TTS6, wherein the phages are optionally encapsulated. The bacteriophage composition can comprise a combination of encapsulated phages and unencapsulated phages in a ratio of about 1:1. In certain embodiments, the composition can comprise the phages TTS1 having a genome comprising a nucleotide sequence of SEQ ID NO: 1, TTS2 having a genome comprising a nucleotide sequence of SEQ ID NO: 2, TTS3 having a genome comprising a nucleotide sequence of SEQ ID NO: 3, TTS4 having a genome comprising a nucleotide sequence of SEQ ID NO: 4, TTS5 having a genome comprising a nucleotide sequence of SEQ ID NO: 5, and TTS6 having a genome comprising a nucleotide sequence of SEQ ID NO: 6, wherein the phages are optionally encapsulated.

A “subject” can be any animal susceptible to infection with Salmonella Gallinarum, such as poultry. Example of poultry includes, but are not limited to, domesticated birds such as chickens, ducks, turkeys or geese, Rock Cornish hen; game birds such as pheasant, squabs, guinea fowl, peafowl, grouse, or quail; and large birds such as ostriches, emus or rheas. In exemplary embodiments, the subject can be a chicken.

An infection caused by Salmonella Gallinarum is fowl typhoid. Fowl typhoid can be acute or chronic and can cause anorexia, diarrhea, dehydration, weakness, and high mortality.

One or more bacteriophage(s) can be combined to form a composition comprising at least about 10 plaque forming units (PFU) to about 10¹³ PFU of each phage per ml of composition, such as from about 10 PFU to 10¹³ PFU or 10 PFU to about 10¹³ PFU of each phage per ml of composition.

As noted above, the therapeutically or prophylactically effective amount of the bacteriophage composition can be administered to the subject. The bacteriophage composition comprises unencapsulated phages and/or encapsulated phages, or a combination thereof. For example, and without limitation, the therapeutically or prophylactically effective amount can be administered to a subject in need of treating or preventing the infection caused by Salmonella Gallinarum. An effective amount of the bacteriophage composition can be administered to the subject prophylactically (e.g., before disease onset) or therapeutically (e.g., concurrently or after disease onset). In certain embodiments, the effective amount can be administered orally to the subject prior to, during, or following Salmonella Gallinarum exposure, which may lead to infection, in a single or multiple administrations. The effective amount can, for example, include about 10 PFU to about 10¹³ PFU of each phage per ml of composition, in volumes between 0.2 mL to 2 mL per bird per treatment.

In certain embodiments, a bacteriophage composition can be administered to a subject at about 10 PFU to about 10¹³ PFU of each phage per ml of composition, in volumes between 0.2 mL to 2 mL per bird per treatment. The bacteriophage composition can be administered at least once, twice, thrice or more times daily. Suitably, the bacteriophage composition can be administered over a period of 0 to 60 days.

The subject can have an infection such as fowl typhoid caused by Salmonella Gallinarum. The effective amount of the bacteriophage composition can be administered to the subject orally. As used herein, the term “administering” and its formatives generally refer to any and all means of introducing a composition to the subject, including, but not limited to, by oral, topical, intravenous, subcutaneous, and like routes of administration. In exemplary embodiments, the bacteriophage composition can be administered by oral gavage using a Monoject Curved Tip Syringe. The bacteriophage composition can be administered as a capsule or in the form of feed or water.

In certain embodiments, a bacteriophage composition comprises an effective amount of one or more bacteriophages selected from TTS1, TTS2, TTS3, TTS4, TTS5, and TTS6, and a pharmaceutically acceptable carrier, excipient, diluent, or combinations thereof.

Effective amounts can range, for example, from about 10 PFU to 10¹³ PFU of each phage per ml of composition, in volumes between 0.2 mL to 2 mL per bird per treatment. The total effective amount of the bacteriophage composition can be administered in single or divided doses and can, at the practitioner's discretion, fall outside of the typical range given herein.

Six wild-type Salmonella Gallinarum phages were isolated to develop a phage-based treatment for Salmonella Gallinarum infections in a subject. The suitable subject can be a chicken. The phages were isolated from wastewater samples obtained from wastewater treatment facilities in central Indiana (US) or wastewater and poultry samples from the peri-Lahore (Pakistan) area using the Salmonella Gallinarum monocultures and the methods as described in Wall et al., 2010, Applied and Environmental Microbiology, 76(1), 48-53, which is hereby specifically incorporated by reference for its teachings regarding same.

In certain embodiments, phages can be characterized by their families. TTS1, TTS3, TTS4, TTS5, and TTS6 belong to Myoviridae family, and TTS2 belongs to Podoviradae family. Transmission electron microscopy (TEM) studies showed that these phages belonged to Myoviridae (n=5) and Podoviradae (n=1) families. Characteristics of Salmonella Gallinarum phages chosen for characterization and animal trials are discussed in Table 1. The phages were further characterized in terms of their viability in different environments such as low pH, simulated gastric fluids (SGF), and host-range. The genome of each phage was also sequenced.

TABLE 1

| | | | Tail | Head | |
|---|---|---|---|---|---|
| | SEQ ID | Isolation | Size | Size | |
| Phage | number | Location | (nm) | (nm) | Family |
| vB_SalM- | SEQ ID | Pakistan | 168.7 ± | 74.1 ± | Myoviridae |
| TTS1 (TTS1) | NO: 1 | | 13.7 | 6.0 | |
| vB_SalP- | SEQ ID | Pakistan | 19.0 ± | 55.5 ± | Podoviradae |
| TTS2 (TTS2) | NO: 2 | | 3.4 | 2.5 | |
| vB_SalM- | SEQ ID | Pakistan | 145.6 ± | 58.1 ± | Myoviridae |
| TTS3 (TTS3) | NO: 3 | | 9.3 | 4.6 | |
| vB_SalM- | SEQ ID | US | 125.1 ± | 57.1 ± | Myoviridae |
| TTS4 (TTS4) | NO: 4 | | 2.3 | 1.0 | |
| vB_SalM- | SEQ ID | US | 153.9 ± | 61.7 ± | Myoviridae |
| TTS5 (TTS5) | NO: 5 | | 6.7 | 1.2 | |
| vB_SalM- | SEQ ID | US | 124.7 ± | 59.1 ± | Myoviridae |
| TTS6 (TTS6) | NO: 6 | | 5.0 | 2.8 | |

The degree of lysis of the isolated phages was identified against the library of ten Salmonella Gallinarum strains. The degree of lysis was assessed using a four-point scale 0=no lysis; 1=some lysis; 2=moderate lysis; 3=complete lysis. The lytic capacity of each phage was assessed by combining phages with a log-phase growth cultures of the targeted Salmonella Gallinarum strain in LB broth and measuring bacterial concentrations over time in triplicate as described in Hong et al., 2014, Journal of Animal Science, 92 (4), 1366-1377 which is hereby specifically incorporated by reference for its teachings regarding same. The results are summarized in Table 2. TTS1, TTS3, and TTS4 lysed all 10 strains of Salmonella Gallinarum while TTS2 (90%), TTS5, TTS6 each lysed eight of 10 strains. Spectrum analysis revealed that each phage lysed at least 8 out of 10 different strains of Salmonella Gallinarum and significantly reduced (P<0.05) Salmonella Gallinarum when co-cultured in liquid medium with Salmonella Gallinarum.

TABLE 2

| Salmonella | | | | | | |
|---|---|---|---|---|---|---|
| Gallinarum | Salmonella Gallinarum Phages | | | | | |
| Strains | TTS1 | TTS2 | TTS3 | TTS4 | TTS5 | TTS6 |
| 51 | 3 | 3 | 3 | 3 | 3 | 0 |
| 53 | 3 | 1 | 3 | 3 | 0 | 3 |
| 54 | 3 | 3 | 3 | 3 | 3 | 3 |
| 75 | 3 | 0 | 3 | 1 | 3 | 3 |
| 18-1 | 2 | 3 | 3 | 3 | 3 | 3 |
| 52B | 3 | 3 | 3 | 3 | 3 | 3 |
| 52-C | 3 | 3 | 1 | 3 | 3 | 3 |
| 24B | 3 | 3 | 3 | 2 | 3 | 3 |
| 52-A | 3 | 3 | 3 | 3 | 0 | 0 |
| 24-A | 3 | 1 | 3 | 3 | 3 | 3 |
| Lytic range | 100 | 90 | 100 | 100 | 80 | 80 |
| (%) | | | | | | |

0 = no lysis; 1 = some lysis; 2 - moderate lysis; 3 - complete lysis

Phages were encapsulated in sodium alginate-CaCl₂). Encapsulation efficiency (EE %) for each of the six phages is as shown in Table 3. Encapsulation efficiency was calculated by comparing the concentration of encapsulated phages to the concentration of the original phage stock before encapsulation:

$$EE \% = \frac{Concentaration of encapsulated bacteriophages}{initial concentration of unprotected bacteriophages} \times 100$$

The highest EE % was recorded for TTS2 bacteriophage (99.3%) followed by TTS5 (96.8%), TTS6 (96.7%), TTS3 (94.5%), TTS4 (93.4%), and then TTS1 (90.6%).

TABLE 3

| | Encapsulated | Unencapsulated | |
|---|---|---|---|
| | phage | phage | |
| | concentration | concentration | Encapsulation |
| Phage | (log PFU/g) | (log PFU/mL) | efficiency (%) |
| TST1 | 9.07 ± 0.16 | 10.01 ± 0.12 | 90.6 |
| TTS2 | 9.71 ± 0.02 | 9.78 ± 0.02 | 99.3 |
| TTS3 | 9.87 ± 0.02 | 10.5 ± 0.09 | 94.5 |
| TTS4 | 9.85 ± 0.02 | 10.5 ± 0.14 | 93.4 |
| TTS5 | 10.18 ± 0.02 | 10.5 ± 0.14 | 96.8 |
| TTS6 | 9.08 ± 0.10 | 9.39 ± 0.03 | 96.7 |

The potential impact of the gastric environment and intestinal environment on the viability of both unencapsulated phages and encapsulated phages was measured in quadruplicate by incubating each phage in simulated gastric fluid (SGF; 0.3% pepsin, 0.85% NaCl in H₂O, pH=2.5) and simulated intestinal fluid (SIF; 0.1% bile salt, 0.4% pancreatin in 50 mM KH₂PO₄, pH~6.8) as described in Colom et al., 2017a, Scientific Reports, 7. Rate of release of phages from encapsulated beads in SGF was also determined.

Although SGF reduced the viability of unencapsulated phages to undetectable levels after only 5 min exposure, encapsulated phages remained 100% viable in SGF after 30 min exposure to SGF. However, no appreciable changes were observed in the viability of unencapsulated phages even when exposed to SIF for 180 minutes. Sequential incubation of encapsulated phages in SGF (30 min) followed by SIF (180 min) indicated that maximum release of phages from capsules occurred within 60 minutes of SIF exposure.

Animal trials were conducted to test the efficacy of phages. The initial phage treatment and Salmonella Gallinarum challenge were administered simultaneously in the first animal trial. Birds were challenged with Salmonella Gallinarum. In the second animal trial, the birds were challenged with higher concentrations of Salmonella Gallinarum. In the first animal trial, 7-day-old chicks were challenged with Salmonella Gallinarum orally (500 μL at 5×10⁶ CFU) and treated with either unencapsulated phages or encapsulated phages (500 μL at 5×10⁸ PFU/mL or g) on hours 0, 12, and 24 post-challenge. Challenged but untreated and unchallenged/untreated birds were included as controls. At 1 day post challenge (dpc), birds treated with unencapsulated and encapsulated phages had significantly lower (P<0.05) cecal Salmonella concentrations (4.36±0.20 and 5.05±0.22 log CFU/g, respectively) compared to untreated birds (5.71±0.13 log CFU/g). At 4 dpc, Salmonella Gallinarum concentrations in the ceca of birds treated with encapsulated and unencapsulated phages were significantly lower (P<0.05; 3.26±0.62 and 4.02±0.15 log CFU/g, respectively) compared to untreated birds (4.65±0.08 log CFU/g). In a second animal trial, birds were challenged with a higher dose of Salmonella Gallinarum (1 mL at 1×10⁹ CFU) and treated with encapsulated phages. Salmonella concentrations in the ceca of birds treated with unencapsulated phages, encapsulated phages, and a mixture of unencapsulated and encapsulated phages were significantly lower (4.28±0.11, 3.72±0.40, and 3.81±0.36 log CFU/g, respectively) than those found in untreated birds (5.26±0.19 log CFU/g). At 2 dpc, concentrations of Salmonella Gallinarum in ceca of birds treated with unencapsulated, encapsulated, and mix phages were significantly lower (4.31±0.53, 3.96±0.61, and 4.38±0.44 log CFU/g, respectively) than those found in untreated birds (5.72±0.27 log CFU/g). However, no significant differences were found in concentrations of Salmonella Gallinarum in the ceca of birds treated with encapsulated phages versus those treated with unprotected phages or a mixture of encapsulated and unencapsulated phages. Similarly, at 4 dpc, Salmonella Gallinarum concentrations in the ceca of birds treated with unencapsulated phages, encapsulated phages, and a mixture of unencapsulated and encapsulated phages were significantly lower (3.17±0.45, 3.56±0.51, and 3.81±0.54 log CFU/g, respectively) than found in those of untreated birds (5.79±0.08 log CFU/g). At 7 dpc, concentrations of Salmonella Gallinarum in the ceca of birds treated with a mixture of unencapsulated and encapsulated phages were 2.40=0.55 log CFU/g and significantly lower (P<0.05) than those found in the ceca of untreated birds (7.08±0.19 log CFU/g) and those found in the ceca of birds treated with unencapsulated or encapsulated phages. However, concentrations of Salmonella Gallinarum in the ceca of birds treated with encapsulated and unencapsulated phages were significantly lower (P<0.05; 4.29±0.39 and 4.60±0.37 log CFU/g, respectively) than those found in untreated birds (7.08±0.19 log CFU/g).

The results of the experiments indicate that this bacteriophage treatment reduces Salmonella Gallinarum concentrations in chickens.

### EXPERIMENTAL

### Bacterial Strains

Ten Salmonella enterica serovar Gallinarum strains were provided by Lahore, Pakistan. The strains were isolated from diseased chickens from different poultry farms in the vicinity of Lahore, Pakistan.

### Isolation of Phage

Wastewater samples (50 mL) were first centrifuged (20 min at 3800×g) to sediment solids. The supernatant was then filtered through 0.45 micron filters (Corning Inc., Corning, NY). Filtered samples were then enriched by combining filtrate (20 mL) with 20 mL of log phase growth Salmonella Gallinarum cultures in 2× Luria Bertani (LB) broth (20 g tryptone, 10 g NaCl, and 10 g yeast extract in 1 L H₂O) and incubated overnight at 37° C. with shaking. Overnight cultures were again centrifuged and filtered as previously described above. Phages were isolated from the supernatant by standard plaque assay by first creating 10-fold serial dilutions of the supernatant in phosphate buffer saline (PBS; 8 g NaCl, 0.2 g KCl, 1.14 g Na₂HPO₄, 0.24 g KH₂PO₄ in 1 L H₂O, pH~7.4). Aliquots (100 μL) of the dilutions were then combined with 67.5 μL of 1M CaCl₂, 3.5 mL LB overlay, and 100 μL of log-phase Salmonella Gallinarum culture. The solutions were mixed, applied to the top of LB agar plates, and incubated overnight at 37° C. Individual phage plaques were purified by lifting plaques from the agar plates and suspending the plaques in PBS, serially diluting the phage/PBS solution, and re-plating on LB medium. The purification steps were repeated an additional two times. Stock solutions of isolated phages were created by combining purified phage samples (1 mL) with 500 mL of LB broth containing log-phase Salmonella Gallinarum and incubating overnight at 37° C. with shaking. The overnight culture was centrifuged and filtered as described above, and stocks were stored at 4° C. This method resulted in the isolation and purification of 13 bacteriophages lytic against Salmonella Gallinarum.

### Spectrum Analysis

The host range of each isolated phage was tested against a library of 10 Salmonella Gallinarum strains isolated from diseased chickens in Pakistan using a spot-assay method in duplicate as described in Sundarram et al., 2021, Foodborne Pathogens and Disease, 18 (6), 413-418 which is hereby specifically incorporated by reference for its teachings regarding same. Individual Salmonella Gallinarum strains were grown to log-phase in LB broth. The log-phase culture (100 μL) was combined with 4 mL of LB-overlay and 67.5 μL of 1 M CaCl₂), mixed briefly, and added to the top of LB agar plates. Individual phages were then applied to the top of the overlay in 7 μL aliquots (spots). Spots containing only PBS or sterile H₂O were also applied as negative controls. Spotted plates were incubated overnight at 37° C. and visualized for lysis (spots). The degree of lysis was assessed using a four-point scale (0=no lysis; 1=some lysis; 2=moderate lysis; 3=complete lysis).

### Phage Morphology

Individual phages were classified as to family by visualization using TEM at the Purdue University Electron Microscopy Facility (West Lafayette, IN USA). Samples were fixed with 2.5% glutaraldehyde in 0.1 M sodium cacodylate buffer and rinsed before negative staining with 1% phosphotungstic acid. Images were acquired on a FEI Tecnai G2 20 electron microscope equipped with a LaB6 source and operating at 200 kV.

### Lytic Capacity

The lytic capacity of each phage was assessed by combining phages with a log-phase growth cultures of the targeted Salmonella Gallinarum strain in LB broth and measuring bacterial concentrations over time in triplicate as described in Hong et al. 2014, Journal of Animal Science, 92(4), 1366-1377. Briefly, Salmonella cultures were incubated in LB broth at 37° C. to reach an optical density of OD600=0.2. Phages were then added to the cultures at an MOI=10.0, and the solution was incubated with shaking at 37° C. Optical density of the culture was measured at 1, 2, 3, 4, 8, and 24 hr following application of phages. Cultures containing only bacteria (no phage application) and cultures containing only bacteria and antibiotics (chloramphenicol, gentamycin, kanamycin, tetracycline, or vancomycin) at minimum inhibitory concentrations (MICs) reported by the National Antibiotic Resistance Monitoring System (NARMS 2019) were included as controls.

### Encapsulation

Phages were microencapsulated using the BUCHI B-390 Encapsulator (Buchi Corporation, New Castle, DE USA). Phage solutions (500 mL) were combined with 1.5% sodium alginate and mixed by shaking at room temperature. The phage/sodium alginate solution was passed through an aperture of 300 μm at 300-750 mbar pressure. The droplets produced were collected in a hardening solution (100 mM CaCl₂)), and the resulting beads were filtered through Whatman filters (size 1; Tisch Scientific, North Bend, OH, USA) to remove residual CaCl₂) and stored at 40 C in 10 mM MgSO4 until further use. Encapsulation efficiency (EE %) was measured in triplicate by dissolving 0.5 g encapsulated phages in 4.5 mL of dissolving solution (0.2M sodium bicarbonate, 50 mM sodium citrate, 50 mM Tris HCl, pH=7.5). Dissolved solutions were then serially diluted, and phages were quantified by plaque assay as described above.

### Survivability in Gastric Environments

Phage concentrations were adjusted to 10⁹ PFU/mL in either SGF and incubated at 41° C. with gentle shaking. Aliquots (100 μL) were collected from each sample at 0, 5, 15, and 30 min, and phage concentrations were measured by standard plaque assay as described above. Samples containing only phage and PBS were included as controls.

Viability was measured as:

$$Viable \% = \frac{Concentration of phage after exposed to SGF for specific time interval}{Concentration of phage after exposed to PBS for specific time interval} \times 100$$

### Survivability in Intestinal Environments

The potential impact of the intestinal environment on the viability of unencapsulated phages was measured in quadruplicate by incubating each phage in simulated intestinal fluid (SIF; 0.1% bile salt, 0.4% pancreatin in 50 mM KH₂PO₄, pH~6.8) as previously described with some modifications. Briefly, 0.5 mL of each phage was added to 4.5 mL of prewarmed SIF and incubated at 41° C. with gentle shaking. Aliquots (100 μL) were collected from each sample at 0, 60, and 180 min, and phage concentrations were measured by standard plaque assay as described above. Phage concentrations at described times were compared to each other to assess the impact of the intestinal environment on phages over time.

Rate of Release of Phages from Encapsulated Beads

To measure the rate of release of phages from encapsulated beads, 0.5 g of encapsulated phages were incubated in 4.5 mL of SGF for 30 minutes in duplicate. The beads were then washed with SM buffer (100 mM NaCl, 8 mM MgSO₄·7H₂O, 50 mM Tris-Cl in 1 L of H₂O) and transferred to 4.5 mL of SIF. Samples (100 μL) were collected at 0, 5, 30, 60, and 120 min, and phage concentrations were measured by standard plaque assays as described above. The released phage concentration was calculated by summing phage concentrations at each time value.

### Animal Trial 1

Animal trials were conducted in BSL-2 facilities at Purdue University (West Lafayette, IN). Day-old Jumbo Cornish broiler chicks were purchased from Townline Hatchery (Zeeland, MI USA). Chicks (100) were randomly distributed to four pens (25 chicks per pen) in four separate rooms. Chicks were provided feed (non-antibiotic) and water ad libitum and allowed to acclimate to the environment for 6 days. The four pens/rooms corresponded with the four treatments: 1) control 1 (Salmonella Gallinarum challenge, no phage treatment); 2) control 2 (no Salmonella Gallinarum challenge, no phage treatment); 3) Salmonella Gallinarum challenge and treatment with unencapsulated phages (six total phages); and 4) Salmonella Gallinarum challenge and treatment with encapsulated phages (six total phages). Prior to the initiation of treatments, five chicks from each group were euthanized by CO₂ and cervical dislocation, and cecal contents collected to assess baseline Salmonella Gallinarum carriage rates across the groups. Cecal contents were serially diluted and the dilutions plated on Brilliant Green Agar medium (BGA; Neogen, East Lansing, MI USA). At 7 d of age, chicks receiving the bacterial challenge were administered 5×10⁶ CFU (500 μL) of Salmonella Gallinarum 1-18 by oral gavage using a Monoject Curved Tip Syringe (Sigma Aldrich, St. Louis, MO USA). Chicks in phage treatment groups were administered phages (500 μL; 10⁸ PFU/mL) by oral gavage using a Monoject Curved Tip Syringe (Sigma Aldrich, St. Louis, MO USA) at 0, 12, and 24 hr post-challenge. Six birds from each treatment group were euthanized by CO₂ and cervical dislocation at 1, 2, and 4 d post-challenge. Cecal Salmonella Gallinarum concentrations were measured by homogenizing ceca (tissue and contents) in peptone water (Difco, Sparks, MD, USA), serially diluting the homogenized samples, plating on BGA medium containing 25 μg/mL streptomycin, and incubating overnight at 37° C. Presumptive Salmonella Gallinarum colonies were confirmed biochemically using Lysine Iron Agar (LIA; Difco) and Triple Sugar Iron agar (TSI: Difco).

### Animal Trial 2

Animal trial 2 was also conducted in BSL-2 facilities at Purdue University (West Lafayette, IN). Day-old Jumbo Cornish broiler chicks were purchased from Townline Hatchery (Zeeland, MI USA). Chicks (170) were randomly distributed to five pens (34 chicks per pen) in four separate rooms. Chicks were provided feed (non-antibiotic) and water ad libitum and allowed to acclimate to the environment for 6 days. The five pens corresponded with the five treatments: 1) control 1 (Salmonella Gallinarum challenge, no phage treatment); 2) control 2 (no Salmonella Gallinarum challenge, no phage treatment); 3) Salmonella Gallinarum challenge and treatment with unencapsulated phage; 4) Salmonella Gallinarum challenge and treatment with encapsulated phages (six total phages); and 5) Salmonella Gallinarum challenge and treatment with mixture (1:1) of unencapsulated and encapsulated phage. Birds in treatment groups 4 and 5 were kept in separate pens but in same room. Prior to the initiation of treatments, six chicks from each group were euthanized by CO2 and cervical dislocation and cecal contents collected to assess baseline Salmonella Gallinarum carriage rates across the groups. Cecal contents were serially diluted and the dilutions plated on BGA media. At 14 d of age, chicks receiving the bacterial challenge were administered 10⁹ CFU (1 mL) of Salmonella Gallinarum 1-18 by oral gavage using an oral gavage gun (Neogen). Chicks in phage treatment groups were administered phages (1 mL; 1010 PFU/mL) by oral gavage (an oral gavage gun was used for unencapsulated phages and a Monoject Curved Tip Syringe was used for encapsulated phages) at −2, −1, 0, 1, 2, and 3 d post-challenge. Six birds from each treatment group were euthanized by CO2 and cervical dislocation at 1, 2, 4 and 7 d post-challenge. Cecal Salmonella Gallinarum concentrations were measured by homogenizing cecal contents in peptone water, serially diluting the homogenized samples, plating on BGA medium, and incubating overnight at 37° C. Presumptive Salmonella Gallinarum colonies were confirmed biochemically using Lysine Iron Agar (LIA; Difco) and Triple Sugar Iron agar (TSI: Difco). Rates of re-isolation of Salmonella Gallinarum from liver and spleen samples of birds was also measured by placing tissues in peptone water and incubating overnight at 37° C. The overnight culture (100 μL) was inoculated into 5 mL of Rappaport Vassiliadis broth [Sigma chemical, MO, USA], incubated overnight at 37° C., plated on BGA medium, and again incubated overnight 37° C. overnight. Presumptive Salmonella Gallinarum colonies were confirmed as described above.

### Statistical Analysis

Data from animal trials and simulated intestinal fluid trials were analyzed using the GLM procedure of SAS (SAS Institute Inc., Cary, NC, USA). The model included the treatment as a fixed effect. The least square means of each treatment were calculated, and differences among least square means were tested using the PDIFF option with Tukey's adjustment. For simulated gastric fluid trials, paired t-tests were used to compare control and treatment values (concentration after SGF exposure) of each time interval. Differences were considered statistically significant at P<0.05.

### Results

### Impact of Gastric Environment on Unencapsulated and Encapsulated Phages

At 0 min exposure to SGF, TTS1 showed higher viability (79.6% viable) than TTS3 (76.4%), TTS5 (52.3%), TTS2 (48.2%), TTS4 (32.77%), and TTS6 (20.7%). After 5 min of exposure to SGF, TTS5, TTS3, and TTS4, viability was reduced to 0.0% (no plaques detected), while viability of TTS1, TTS6, and TTS2 was 23.2, 14.6, and 14.4%, respectively. No phages, however, were detected in any sample by the next time point (15 min). Incubation of the same phages in PBS for the same time periods did not affect phage concentrations. As such, phages viability when exposed to SGF was significantly lower (P<0.05) than when exposed to only PBS at all time points. In contrast, when encapsulated phage were exposed to SGF, TTS6 phage showed higher viability (102.0% viable) than TTS3 (100.6%), TTS4 (99.8%), TTS5 (99.5%), TTS 2 (99.0%), and TTS1 (97.9%) at 0 min exposure. After 5, 15 and 30 min of exposure to SGF, viability of encapsulated phages ranged between 97.4-103.0% (Table 4). Statistical analysis showed that at most time intervals, differences between concentrations of viable phages exposed to SGF were not different from concentrations of phages exposed to only PBS (P>0.05).

TABLE 4
Viability of encapsulated Salmonella Gallinarum phages after exposure to simulated gastric fluid
(SGF) for 0, 5, 15, 30 min

| | Exposure | SGF | PBS | | |
|---|---|---|---|---|---|
| | time | (log PFU/mL; | (log PFU/mL; | Viability | |
| Phage | (min) | Average ± SE) | Average ± SE) | % | P-value |
| TTS1 | 0 | 9.01 ± 0.02 | 9.2 ± 0.01 | 97.9 | 0.80 |
| | 5 | 8.99 ± 0.03 | 9.08 ± 0.01 | 99.0 | 0.11 |
| | 15 | 8.93 ± 0.03 | 9.1 ± 0.01 | 98.6 | 0.06 |
| | 30 | 8.98 ± 0.01 | 9.05 ± 0.10 | 99.2 | 0.08 |
| TTS2 | 0 | 8.77 ± 0.03 | 8.86 ± 0.06 | 99.0 | 0.23 |
| | 5 | 8.78 ± 0.07 | 8.94 ± 0.80 | 98.2 | 0.16 |
| | 15 | 8.68 ± 0.03 | 8.90 ± 0.30 | 97.5 | 0.01 |
| | 30 | 8.73 ± 0.04 | 8.96 ± 0.30 | 97.4 | 0.03 |
| TTS3 | 0 | 9.70 ± 0.08 | 9.64 ± 0.05 | 100.6 | 0.70 |
| | 5 | 9.69 ± 0.03 | 9.65 ± 0.02 | 100.9 | 0.36 |
| | 15 | 9.62 ± 0.02 | 9.60 ± 0.03 | 100.2 | 0.68 |
| | 30 | 9.58 ± 0.03 | 9.66 ± 0.05 | 99.17 | 0.13 |
| TTS4 | 0 | 9.00 ± 0.06 | 9.01 ± 0.05 | 99.8 | 0.75 |
| | 5 | 8.96 ± 0.07 | 8.98 ± 0.06 | 99.8 | 0.37 |
| | 15 | 8.93 ± 0.07 | 8.97 ± 0.06 | 99.5 | 0.34 |
| | 30 | 8.89 ± 0.05 | 8.97 ± 0.06 | 99.1 | 0.14 |
| TTS5 | 0 | 8.97 ± 0.05 | 9.02 ± 0.02 | 99.5 | 0.06 |
| | 5 | 8.94 ± 0.03 | 9.00 ± 0.01 | 99.3 | 0.30 |
| | 15 | 8.84 ± 0.04 | 8.95 ± 0.01 | 98.8 | 0.04 |
| | 30 | 8.80 ± 0.03 | 8.93 ± 0.03 | 98.5 | 0.01 |
| TTS6 | 0 | 9.11 ± 0.01 | 8.91 ± 0.02 | 102.0 | 0.007 |
| | 5 | 9.01 ± 0.03 | 8.95 ± 0.01 | 100.0 | 0.27 |
| | 15 | 9.13 ± 0.04 | 8.92 ± 0.03 | 103.0 | 0.01 |
| | 30 | 8.89 ± 0.03 | 8.84 ± 0.01 | 100.5 | 0.29 |

### Impact of Intestinal Environment on Phages

Phage stability in SIF was assessed by comparing phage concentrations after 0, 60, and 180 min of exposure to SIF. Phage concentrations did not differ from each other (P>0.05) at 0, 60, and 180 min exposure to SIF for any phage. For phage TTS6, phage concentrations were 8.75±0.03, 8.76±0.02, and 8.70±0.03 log PFU/mL at exposure time of 0, 60 and 180 minutes to SIF, respectively (P=0.42). Similar patterns were observed for all other phages (Table 5). While slight changes in phages concentration (0.01 to 0.5 PFU/mL) were observed, these differences were not statistically significant. Viability of encapsulated phage in SIF was not tested as it was concluded that SIF had no impact on viability of unencapsulated phages.

TABLE 5
Viability of unencapsulated Salmonella Gallinarum phages after exposure to simulated
intestinal fluid (SIF) for 0, 60, 180 min.

| | 0 min | 60 min | 180 min | | |
|---|---|---|---|---|---|
| | (log PFU/mL; | (log PFU/mL; | (log PFU/mL; | | |
| Phage | Average ± SE) | Average ± SE) | Average ± SE) | SEM | P-value |
| TTS1 | 9.74 ± 0.06 | 9.79 ± 0.03 | 9.80 ± 0.04 | 0.04 | 0.68 |
| TTS2 | 9.58 ± 0.01 | 9.56 ± 0.02 | 9.50 ± 0.04 | 0.03 | 0.15 |
| TTS3 | 10.79 ± 0.01 | 10.79 ± 0.05 | 10.75 ± 0.06 | 0.05 | 0.74 |
| TTS4 | 8.77 ± 0.01 | 8.77 ± 0.02 | 8.70 ± 0.05 | 0.04 | 0.26 |
| TTS5 | 8.99 ± 0.05 | 8.88 ± 0.05 | 8.88 ± 0.06 | 0.05 | 0.30 |
| TTS6 | 8.75 ± 0.03 | 8.76 ± 0.02 | 8.70 ± 0.03 | 0.03 | 0.42 |

### Release of Encapsulated Phages

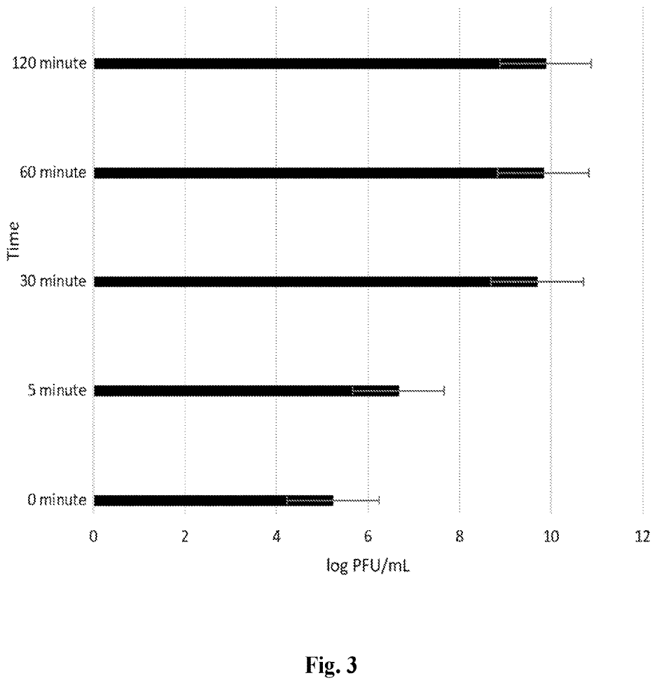

To measure the rate of liberation of encapsulated phages from their beads, encapsulated phages were sequentially incubated in SGF (30 min) and then in SIF (120 min) to mimic transit time of the chicken's gastrointestinal tract (FIG. 3). At the time when phages were transferred from SGF to SIF, phages were released at 5.23 log PFU/mL. After 5 min of incubation in SIF, concentrations of released phages reach 6.66 log PFU/mL and 9.69 log PFU/mL at 30 min of incubation in SIF. Concentrations of released phages remained constant at 60 and 120 min of incubation (9.83 and 9.86 log PFU/mL, respectively).

### Animal Trial 1

At 1 d post-challenge, concentrations of Salmonella Gallinarum in the ceca of birds treated with unencapsulated phages (4.36±0.20 log CFU/g) were significantly lower (P<0.05) than those found in the ceca of untreated birds (5.71±0.13 log CFU/g; Table 6). Likewise, concentrations of Salmonella Gallinarum in birds treated with encapsulated phages (5.05±0.22 log CFU/g) were significantly lower than in ceca of untreated birds (P<0.05). At 2 d post-challenge, concentrations of Salmonella Gallinarum in the ceca of birds treated with encapsulated and unencapsulated phages (4.53±0.15 and 4.29±0.20 log CFU/g, respectively) were significantly lower (P<0.05) than those found in ceca of untreated birds (5.59±0.14 log CFU/g). However, no significant differences were found in concentrations of Salmonella Gallinarum in the ceca of birds treated with encapsulated versus unencapsulated phages. At 4 d post-challenge, concentrations of Salmonella Gallinarum in the ceca of birds treated with encapsulated and unencapsulated phages (3.26±0.62, 4.02±0.15 log CFU/g, respectively) were significantly lower (P<0.05) than those found in ceca of untreated birds (4.65±0.08 log CFU/g; P<0.05).

TABLE 6
Concentrations of Salmonella Gallinarum (log CFU/g) in the ceca of
birds treated with unencapsulated or encapsulated phages at 1, 2, and
4 d post-challenge (dpc; Trial 1)

| Treatment | 1 dpc | 2 dpc | 4 dpc |
|---|---|---|---|
| unencapsulated | 4.36 ± 0.20 ^{c} | 4.29 ± 0.20 ^{b} | 4.02 ± 0.15 ^{ab} |
| Phage | | | |
| Encapsulated Phage | 5.05 ± 0.22 ^{b} | 4.53 ± 0.15 ^{b} | 3.26 ± 0.62 ^{b} |
| Control 1 | 5.71 ± 0.13 ^{a} | 5.59 ± 0.14 ^{a} | 4.65 ± 0.08 ^{a} |
| Control 2 | 0.00 | 0.00 | 0.00 |
| P-value | <0.001 | <0.001 | 0.003 |

Control 1 birds received Salmonella Gallinarum challenge and no phage treatment; Control 2 birds received neither bacterial challenge nor phage treatment; numbers with different superscripts are statistically different at P<0.05. Comparisons are within dpc.

### Animal Trial 2

At 1 dpc, Salmonella Gallinarum concentrations in the ceca of birds treated with unencapsulated phages, encapsulated phages, and a mixture of unencapsulated and encapsulated phages were significantly lower (4.28±0.11, 3.72=0.40, and 3.81±0.36 log CFU/g, respectively) than those found in untreated birds (5.26±0.19 log CFU/g; P<0.05; Table 7). At 2 d post-challenge, concentrations of Salmonella Gallinarum in the ceca of birds treated with unencapsulated, encapsulated and a mixture of unencapsulated and encapsulated phages (4.31±0.53, 3.96=0.61, and 4.38±0.44 log CFU/g, respectively) were significantly lower (P<0.05) than those found in the ceca of untreated birds (5.72±0.27 log CFU/g). However, no significant differences were found in concentrations of Salmonella Gallinarum in the ceca of birds treated with encapsulated versus those treated with unencapsulated phages or a mixture of encapsulated and unencapsulated phages. Similarly, at 4 dpc, Salmonella Gallinarum concentrations in the ceca of birds treated with unencapsulated phages, encapsulated phages, and a mixture of unencapsulated and encapsulated phages (3.17±0.45, 3.56±0.51, and 3.81±0.54 log CFU/g, respectively) were significantly lower than those found in untreated birds (5.79±0.08 log CFU/g, P<0.05). At 7 dpc, concentrations of Salmonella Gallinarum in the ceca of birds treated with mixture of unencapsulated and encapsulated phages (2.40±0.55 log CFU/g) were significantly lower (P<0.05) than those found in the ceca of untreated birds (7.08±0.19 log CFU/g) and those found in birds treated with either encapsulated or unencapsulated phages. However, concentrations of Salmonella Gallinarum in the ceca of birds treated with encapsulated and unencapsulated phages (P<0.05; 4.29±0.39 and 4.60±0.37 log CFU/g, respectively) were significantly lower (P<0.05) than those found in untreated birds (7.08±0.19 log CFU/g).

TABLE 7
Concentrations of Salmonella Gallinarum (log CFU/g of cecal content) in the ceca of birds
treated with unencapsulated phages, encapsulated phages, or a mixture of encapsulated phages
and unencapsulated phages on 1, 2, 4, and 7 days post challenge (dpc; Trial)

| Treatment | 1 dpc | 2 dpc | 4 dpc |
|---|---|---|---|
| unencapsulated | 4.36 ± 0.20 ^{c} | 4.29 ± 0.20 ^{b} | 4.02 ± 0.15 ^{ab} |
| Phage | | | |
| Encapsulated Phage | 5.05 ± 0.22 ^{b} | 4.53 ± 0.15 ^{b} | 3.26 ± 0.62 ^{b} |
| Control 1 | 5.71 ± 0.13 ^{a} | 5.59 ± 0.14 ^{a} | 4.65 ± 0.08 ^{a} |
| Control 2 | 0.00 | 0.00 | 0.00 |
| P-value | <0.001 | <0.001 | 0.003 |

Control 1 birds received Salmonella Gallinarum challenge and no phage treatment;
Control 2 birds received neither bacterial challenge nor phage treatment; numbers with different superscripts are statistically different at P<0.05. Comparisons are within dpc.

As used herein, the following terms and phrases shall have the meanings set forth below. Unless defined otherwise, all technical and scientific terms used herein have the same meaning as commonly understood to one of ordinary skill in the art.

The term “about” can allow for a degree of variability in a value or range, for example, within 10%, within 5%, or within 1% of a stated value or of a stated limit of a range.

The term “substantially” can allow for a degree of variability in a value or range, for example, within 90%, within 95%, or within 99% of a stated value or of a stated limit of a range.

The term “% identity” or “% sequence identity” in relation to nucleic acid or amino acid sequences designates the level of identity or homology between said sequences and may be determined by techniques known in the art.

The terms “a,” “an,” or “the” are used to include one or more than one unless the context clearly dictates otherwise. The term “or” is used to refer to a nonexclusive “or” unless otherwise indicated. In addition, the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. The terms “including” and “having” are defined as comprising (i.e., open language).

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

1. A bacteriophage composition for preventing or treating an infection caused by Salmonella Gallinarum comprising an effective amount of at least one phage selected from vB_SalM TTS1 (TTS1), vB_SalP TTS2 (TTS2), vB_SalM TTS3 (TTS3), vB_SalM TTS4 (TTS4), vB_SalM TTS5 (TTS5), and vB_SalMTTS6 (TTS6), wherein at least one phage is optionally encapsulated.

2. The bacteriophage composition of claim 1, which comprises encapsulated and unencapsulated phages in a ratio of about 1:1.

3. The bacteriophage composition of claim 1 or 2, which comprises phages TTS1, TTS2, TTS3, TTS4, TTS5, and TTS6.

4. The bacteriophage composition of claim 1, 2, or 3, wherein the encapsulated phages are encapsulated with a natural polymer.

5. The bacteriophage composition of claim 4, wherein the natural polymer is sodium alginate, chitosan, gum, gelatin, collagen, or a combination of two or more thereof.

6. The bacteriophage composition of claim 4, wherein the natural polymer is sodium alginate.

7. A bacteriophage composition for preventing or treating an infection caused by Salmonella Gallinarum comprising an effective amount of at least one isolated phage selected from vB_SalM TTS1 (TTS1) having a genome comprising a nucleotide sequence of SEQ ID NO: 1, vB_SalP TTS2 (TTS2) having a genome comprising a nucleotide sequence of SEQ ID NO: 2, vB_SalM TTS3 (TTS3) having a genome comprising a nucleotide sequence of SEQ ID NO: 3, vB_SalM TTS4 (TTS4) having a genome comprising a nucleotide sequence of SEQ ID NO: 4, vB_SalM TTS5 (TTS5) having a genome comprising a nucleotide sequence of SEQ ID NO: 5, and vB_SalM TTS6 (TTS6) having a genome comprising a nucleotide sequence of SEQ ID NO: 6, wherein at least one phage is optionally encapsulated.

8. The bacteriophage composition of claim 7, which comprises encapsulated and unencapsulated phages in a ratio of about 1:1.

9. The bacteriophage composition of claim 7 or 8, which comprises phages TTS1, TTS2, TTS3, TTS4, TTS5, and TTS6 comprising the nucleotide sequence of SEQ ID Nos: 1 to 6, respectively.

10. The bacteriophage composition of claim 7, 8, or 9, wherein the encapsulated phages are encapsulated with a natural polymer.

11. The bacteriophage composition of claim 10, wherein the natural polymer is sodium alginate, chitosan, gum, gelatin, collagen, or a combination of two or more thereof.

12. The bacteriophage composition of claim 10, wherein the natural polymer is sodium alginate.

13. A method for preventing or treating an infection caused by Salmonella Gallinarum comprises administering to a subject a prophylactically or therapeutically effective amount of a bacteriophage composition comprising at least one phage selected from vB_SalM TTS1 (TTS1), vB_SalP TTS2 (TTS2), vB_SalM TTS3 (TTS3), vB_SalM TTS4 (TTS4), vB_SalM TTS5 (TTS5), and vB_SalM TTS6 (TTS6), wherein at least one phage is optionally encapsulated.

14. The method of claim 13, which comprises encapsulated and unencapsulated phages in a ratio of about 1:1.

15. The method of claim 13 or 14, which comprises phages TTS1, TTS2, TTS3, TTS4, TTS5, and TTS6.

16. The method of claim 13, 14, or 15, wherein the encapsulated phages are encapsulated with a natural polymer.

17. The method of claim 16, wherein the natural polymer is sodium alginate, chitosan, gum, gelatin, collagen, or a combination of two or more thereof.

18. The method of claim 16, wherein the natural polymer is sodium alginate.

19. The method of claim 13, wherein the subject is poultry.

20. The method of claim 13 or 19, wherein the prophylactically or therapeutically effective amount is from about 10 PFU to about 10¹³ PFU of each phage per ml of composition, in volumes between 0.2 mL to 2 mL per bird per treatment.

21. The method of claim 20, wherein the prophylactically or therapeutically effective amount is administered orally, topically, or subcutaneously to the subject.

22. The method of claim 21, wherein the prophylactically or therapeutically effective amount is administered in a suspension, feed, water, a spray, or an injection to the subject.

23. The method of claim 20, wherein the prophylactically or therapeutically effective amount is administered to the subject over a period of time ranging from about 1 to about 60 days.

24. A method for preventing or treating an infection caused by Salmonella Gallinarum comprising administering to a subject a prophylactically or therapeutically effective amount of a bacteriophage composition of any one of claims 1-12.
